(19) United States
(12) Patent Application Publication
(10) Pub. No.: US 2017/0296623 A1
(43) Pub. Date: Oct. 19, 2017

(54) INHIBITORY CHIMERIC ANTIGEN RECEPTOR (iCAR OR N-CAR) EXPRESSING NON-T CELL TRANSDUCTION DOMAIN

(71) Applicant: CELLECTIS, Paris (FR)

(72) Inventors: Alexandre JUILLERAT, New York, NY (US); Philippe DUCHATEAU, Draveil (FR); Laurent POIROT, Paris (FR)

(21) Appl. No.: 15/532,430

(22) PCT Filed: Dec. 17, 2015
(86) PCT No.: PCT/EP2015/080376

§ 371 Date: Jun. 1, 2017

(30) Foreign Application Priority Data
Dec. 17, 2014 (DK) PA201470797
Aug. 11, 2015 (DK) PA201570518

(51) Int. Cl. A61K 38/17; C07K 14/47; C07K 14/705; C07K 14/525; A61K 38/00; C07K 14/00

(52) U.S. Cl. CPC A61K 38/17; C07K 14/70596; C07K 14/525; C07K 14/4748; C07K 14/00; A61K 38/00

(57) ABSTRACT

The invention relates to negative T-cell signal inducing chimeric antigen receptor (N-CAR or i-CAR) and to T-cells comprising such N-CAR as well as a positive T-cell signal inducing CAR (P-CAR) as well as their use in therapy.

### FIELD OF THE INVENTION

The invention relates to negative T-cell signal inducing chimeric antigen receptor (N-CAR or i-CAR) and to T-cells comprising such N-CAR as well as a positive T-cell signal inducing CAR (P-CAR) as well as their use in therapy. In particular, the present invention relates to an immune cell engineered to express at least one N-CAR and at least one P-CAR, wherein the P-CAR binds to a first antigen and activates the immunoresponsive cell (i.e. cytotoxicity) whereas the N-CAR binds to a second antigen and inhibits the immunoresponsive cell (i.e. cytotoxicity) through the signaling of a sequence from a TRAIL receptor or a CD200R1 receptor.

The invention also relates to negative T-cell signal inducing chimeric antigen receptor (N-CAR or i-CAR) and to T-cells comprising such N-CAR as well as a positive T-cell signal inducing CAR (P-CAR) as well as their use in therapy. In particular, the present invention relates to an immune cell engineered to express at least one N-CAR and at least one P-CAR, wherein the P-CAR binds to a first antigen and activates the immunoresponsive cell (i.e. cytotoxicity) whereas the N-CAR binds to a second antigen and inhibits the immunoresponsive cell (i.e. cytotoxicity) through the signaling of a sequence usually expressed in non T cells, provided that said sequence is not an ITSM preferably not a sequence selected in a group consisting of SEQ ID NO:13 (human SIGL8), SEQ ID NO:14 (human SIGL7), SEQ ID NO:17 (human SIGL5), SEQ ID NO:20 (human SIGL9), SEQ ID NO: 21 (human SIGL6), SEQ ID NO:22 (human CD33), SEQ ID NO:26 (human SIG12), SEQ ID NO:31 (human SIG11), SEQ ID NO:32 (human SIG10) and SEQ ID NO:19 (human PECA1).

or

provided that said sequence does not comprise a sequence selected in a group consisting of SEQ ID NO:13 (human SIGL8), SEQ ID NO:14 (human SIGL7), SEQ ID NO:17 (human SIGL5), SEQ ID NO:20 (human SIGL9), SEQ ID NO: 21 (human SIGL6), SEQ ID NO:22 (human CD33), SEQ ID NO:26 (human SIG12), SEQ ID NO:31 (human SIG11), SEQ ID NO:32 (human SIG10) and SEQ ID NO:19 (human PECA1).

This system of “NOT gates” is particularly useful in immunotherapy in order to prevent cytotoxicity towards “off-target” healthy or immune cells.

### INTRODUCTION

Adoptive immunotherapy, which involves the transfer of autologous antigen-specific T cells generated ex vivo, is a promising strategy to treat viral infections and cancer. The T cells used for adoptive immunotherapy can be generated either by expansion of antigen-specific T cells or redirection of T cells through genetic engineering (Park, Rosenberg et al. 2011, Trends Biotechnol 29(11):550-7). Transfer of viral antigen specific T cells is a well-established procedure used for the treatment of transplant associated viral infections and rare viral-related malignancies. Similarly, isolation and transfer of tumor specific T cells has been shown to be successful in treating melanoma.

Novel specificities in T cells have been successfully generated through the genetic transfer of transgenic T cell receptors or chimeric antigen receptors (CARs) (Jena, Dotti et al. 2010, Blood 116(7):1035-44). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and heavy variable fragments of a monoclonal antibody joined by a flexible linker. Binding moieties based on receptor or ligand domains have also been used successfully. The signaling domains for first generation CARs are derived from the cytoplasmic region of the ζCD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010, Blood 116(7):1035-44).

However, despite their unprecedent efficacy for tumor eradication in vivo, CAR T cells can promote acute adverse events after being transferred into patients. Among the well documented adverse events is Graft versus host disease (GvHD), on-target off-tumor activity or aberrant lymphoproliferative capacity due to vector derived insertional mutagenesis. Therefore, there is a need to develop cell specific depletion systems to prevent such deleterious events to occur in vivo.

Recently, inhibitory chimeric antigen receptors (N-CARs) were designed having as objective to put the brakes on T cell function upon encountering off-target cells (Fedorov, V. D., Themeli, M., Sadelain, M, 2013, Sci Transl Med 5 (215). In this paper, the authors designed CLTA-4- and PD-1-based N-CARs which could selectively limit cytokine secretion, cytotoxicity, and proliferation induced through the endogenous T cell receptor or an activating chimeric receptor. They have shown that the initial effect of the N-CAR is temporary, thus enabling T cells to function upon a subsequent encounter with the antigen recognized by their activating receptor.

Proteins containing Immunoreceptor tyrosine-based inhibitory motif (ITIM), immunoreceptor tyrosine-based switch motif (ITSM) and SH2-binding motif are, as non-limiting example, known to play a major role in the inhibition, control and modulation of several signaling pathways in T-cells (e.g. TCR) (Barrow A and Trowsdale J, 2006, Eur J Immunol 36 (7): 1646-53, Sharpe H and Freeman G, 2002, Nature Reviews Immunology, (2) 116-126).

However, when the engineered iCAR or N-CAR T-cells contain a transduction domain from T-cell, it may occur an interference or protein interaction with the transduction domain from wild-type T-cells. In order to circumvent this problem, the inventors have sought engineered inhibitory chimeric antigen receptor (N-CAR) based on non-naturally expressed intracellular domains in T-cell and/or intracellular domain from TRAIL receptors and/or CD200 receptor 1. This N-CAR can be used in “logic NOT gates” systems which are composed of a positive signaling CAR (P-CAR) and an inhibitory Gate receptor (iCAR or N-CAR).

### SUMMARY

The present invention is drawn to apply biology principles such as logic “NOT gate” to immune cell technology in order for the engineered immune cells, in particular T-cells, to be inhibited in case off-tumor targets (healthy cells). In particular, the present invention relates to an inhibitory chimeric antigen receptor (iCAR or N-CAR) which contains an intracellular domain from a receptor involved in transduction signal which is not significantly expressed in natural T-cell and/or from a TRAIL receptor and/or the CD200 receptor. The preferred intracellular domains of the invention have at least 80% identity with the polypeptides of SEQ ID NO: 1 to 36. More preferably an intracellular domain of the CAR P of the invention comprises a sequence selected from the group consisting of SEQ ID NO: 1 to 36.

Another aspect of the invention is the engineered immune cell such as T-cell which expressed both said N-CAR and a positive CAR (P-CAR); their respective extracellular binding domains targeting an off tumor cell (healthy cell) and a tumoral cell.

The present invention also relates to a method of engineering of such N-CAR and isolated immune cell, polynucleotides and vectors encoding said CARs, as well as therapeutic treatment using such engineered immune cell.

### BRIEF DESCRIPTION OF DRAWINGS

FIG. 1: Schematic representation of the architectures (versions V1 to V6, preferably V1, V3 and V5) for the different single-chain car chimeric antigen receptor (scCAR) of the invention.

FIG. 2: Schematic representation of the design of inhibitory Gate receptors (N-CAR): the native signaling modulation receptor is engineered in order to replace the native extracellular topological domain by an extracellular binding domain able to bind specifically to an antigen or cell surface marker of an “off-target” healthy cell.

FIG. 3: P-CAR (P is CD20) driven activation (measured by expression of CD69) of transduced T cells mediated through target cells expressing the CD20 antigen. CD69 is an appropriate marker for measuring T cell activation.

FIG. 4: FIG. 4 shows Ratio of % of target cells antigen P-CAR-high/antigen N-CAR-high and antigen P-CAR-high/antigen N-CAR-low after a co-incubation of 6 h with engineered primary T-cells (three ratio of target/effectors are used: 1/1, 1/3 and 1/10).

### DETAILED DESCRIPTION

| Abreviations | Designation |
|---|---|
| N-CAR Or iCAR | negative chimeric antigen receptor |
| P-CAR | positive chimeric antigen receptor |
| TRAIL | tumor-necrosis-factor related apoptosis inducing ligand |
| CDR | complementarity determining regions |
| scFv | single chain antibody fragment |
| KIR2DL2 CD158B1 NKAT6 | Killer cell immunoglobulin-like receptor 2DL2 (CD158 antigen-like |
| | family member B1) (MHC class I NK cell receptor) (Natural killer- |
| | associated transcript 6) (NKAT-6) (p58 natural killer cell receptor clone |
| | CL-43) (p58 NK receptor CL-43) (CD antigen CD158b1) |
| KIR2DL1 CD158A NKAT1 | Killer cell immunoglobulin-like receptor 2DL1 (CD158 antigen-like |
| | family member A) (MHC class I NK cell receptor) (Natural killer- |
| | associated transcript 1) (NKAT-1) (p58 natural killer cell receptor |
| | clones CL-42/47.11) (p58 NK receptor CL-42/47.11) (p58.1 MHC class-I- |
| | specific NK receptor) (CD antigen CD158a) |
| FCGR2B CD32 FCG2 IGFR2 | Low affinity immunoglobulin gamma Fc region receptor II-b (IgG Fc |
| | receptor II-b) (CDw32) (Fc-gamma RII-b) (Fc-gamma-RIIb) (FcRII-b) (CD |
| | antigen CD32) |
| KIR2DL3 CD158B2 KIRCL23 | Killer cell immunoglobulin-like receptor 2DL3 (CD158 antigen-like |
| NKAT2 | family member B2) (KIR-023GB) (Killer inhibitory receptor cl 2-3) (MHC |
| | class I NK cell receptor) (NKAT2a) (NKAT2b) (Natural killer-associated |
| | transcript 2) (NKAT-2) (p58 natural killer cell receptor clone CL-6) (p58 |
| | NK receptor CL-6) (p58.2 MHC class-I-specific NK receptor) (CD antigen |
| | CD158b2) |
| KIR3DL2 CD158K NKAT4 | Killer cell immunoglobulin-like receptor 3DL2 (CD158 antigen-like |
| | family member K) (MHC class I NK cell receptor) (Natural killer- |
| | associated transcript 4) (NKAT-4) (p70 natural killer cell receptor clone |
| | CL-5) (p70 NK receptor CL-5) (CD antigen CD158k) |
| KIR2DL4 CD158D KIR103AS | Killer cell immunoglobulin-like receptor 2DL4 (CD158 antigen-like |
| | family member D) (G9P) (Killer cell inhibitory receptor 103AS) (KIR- |
| | 103AS) (MHC class I NK cell receptor KIR103AS) (CD antigen CD158d) |
| KIR3DL1 CD158E NKAT3 | Killer cell immunoglobulin-like receptor 3DL1 (CD158 antigen-like |
| NKB1 | family member E) (HLA-BW4-specific inhibitory NK cell receptor) (MHC |
| | class I NK cell receptor) (Natural killer-associated transcript 3) (NKAT- |
| | 3) (p70 natural killer cell receptor clones CL-2/CL-11) (p70 NK receptor |
| | CL-2/CL-11) (CD antigen CD158e) |
| KIR2DL5A CD158F | Killer cell immunoglobulin-like receptor 2DL5A (CD antigen CD158f1) |
| CD158F1 KIR2DL5 | |
| MILR1 C17orf60 MCA32 | Allergin-1 (Allergy inhibitory receptor 1) (Mast cell antigen 32) (MCA- |
| | 32) (Mast cell immunoglobulin-like receptor 1) |
| LILRB4 ILT3 LIR5 | Leukocyte immunoglobulin-like receptor subfamily B member 4 (CD85 |
| | antigen-like family member K) (Immunoglobulin-like transcript 3) (ILT- |
| | 3) (Leukocyte immunoglobulin-like receptor 5) (LIR-5) (Monocyte |
| | inhibitory receptor HM18) (CD antigen CD85k) |
| LILRB3 ILT5 LIR3 | Leukocyte immunoglobulin-like receptor subfamily B member 3 (LIR-3) |
| | (Leukocyte immunoglobulin-like receptor 3) (CD85 antigen-like family |
| | member A) (Immunoglobulin-like transcript 5) (ILT-5) (Monocyte |
| | inhibitory receptor HL9) (CD antigen CD85a) |
| KIR3DL3 CD158Z KIR3DL7 | Killer cell immunoglobulin-like receptor 3DL3 (CD158 antigen-like |
| KIRC1 | family member Z) (Killer cell inhibitory receptor 1) (CD antigen |
| | CD158z) |
| SIGLEC8 SAF2 | Sialic acid-binding Ig-like lectin 8 (Siglec-8) (Sialoadhesin family |
| | member 2) (SAF-2) |
| SIGLEC7 AIRM1 | Sialic acid-binding Ig-like lectin 7 (Siglec-7) (Adhesion inhibitory |
| | receptor molecule 1) (AIRM-1) (CDw328) (D-siglec) (QA79 membrane |
| | protein) (p75) (CD antigen CD328) |
| LILRB5 LIR8 | Leukocyte immunoglobulin-like receptor subfamily B member 5 (CD85 |
| | antigen-like family member C) (Leukocyte immunoglobulin-like |
| | receptor 8) (LIR-8) (CD antigen CD85c) |
| LILRB2 ILT4 LIR2 MIR10 | Leukocyte immunoglobulin-like receptor subfamily B member 2 (LIR-2) |
| | (Leukocyte immunoglobulin-like receptor 2) (CD85 antigen-like family |
| | member D) (Immunoglobulin-like transcript 4) (ILT-4) |
| | (Monocyte/macrophage immunoglobulin-like receptor 10) (MIR-10) |
| | (CD antigen CD85d) |
| SIGLEC5 CD33L2 OBBP2 | Sialic acid-binding Ig-like lectin 5 (Siglec-5) (CD33 antigen-like 2) |
| | (Obesity-binding protein 2) (OB-BP2) (OB-binding protein 2) (CD |
| | antigen CD170) |
| FCRL4 FCRH4 IFGP2 IRTA1 | Fc receptor-like protein 4 (FcR-like protein 4) (FcRL4) (Fc receptor |
| | homolog 4) (FcRH4) (IFGP family protein 2) (hIFGP2) (Immune receptor |
| | translocation-associated protein 1) (CD antigen CD307d) |
| PECAM1 | Platelet endothelial cell adhesion molecule (PECAM-1) (EndoCAM) |
| | (GPIIA′) (PECA1) (CD antigen CD31) |
| SIGLEC9 | Sialic acid-binding Ig-like lectin 9 (Siglec-9) (CDw329) (Protein FOAP-9) |
| UNQ668/PRO1302 | (CD antigen CD329) |
| SIGLEC6 CD33L CD33L1 | Sialic acid-binding Ig-like lectin 6 (Siglec-6) (CD33 antigen-like 1) |
| OBBP1 | (CDw327) (Obesity-binding protein 1) (OB-BP1) (CD antigen CD327) |
| CD33 SIGLEC3 | Myeloid cell surface antigen CD33 (Sialic acid-binding Ig-like lectin 3) |
| | (Siglec-3) (gp67) (CD antigen CD33) |
| FCRL5 FCRH5 IRTA2 | Fc receptor-like protein 5 (FcR-like protein 5) (FcRL5) (BXMAS1) (Fc |
| UNQ503/PRO820 | receptor homolog 5) (FcRH5) (Immune receptor translocation- |
| | associated protein 2) (CD antigen CD307e) |
| FCRL2 FCRH2 IFGP4 IRTA4 | Fc receptor-like protein 2 (FcR-like protein 2) (FcRL2) (Fc receptor |
| SPAP1 | homolog 2) (FcRH2) (IFGP family protein 4) (Immunoglobulin receptor |
| UNQ9236/PRO31998 | translocation-associated protein 4) (SH2 domain-containing |
| | phosphatase anchor protein 1) (CD antigen CD307b) |
| FCRL1 FCRH1 IFGP1 IRTA5 | Fc receptor-like protein 1 (FcR-like protein 1) (FcRL1) (Fc receptor |
| | homolog 1) (FcRH1) (IFGP family protein 1) (hIFGP1) (Immune receptor |
| | translocation-associated protein 5) (CD antigen CD307a) |
| SIGLEC12 SIGLECL1 SLG | Sialic acid-binding Ig-like lectin 12 (Siglec-12) (Sialic acid-binding Ig-like |
| UNQ9215/PRO34042 | lectin-like 1) (Siglec-L1) |
| FCRL3 FCRH3 IFGP3 IRTA3 | Fc receptor-like protein 3 (FcR-like protein 3) (FcRL3) (Fc receptor |
| SPAP2 | homolog 3) (FcRH3) (IFGP family protein 3) (hIFGP3) (Immune receptor |
| | translocation-associated protein 3) (SH2 domain-containing |
| | phosphatase anchor protein 2) (CD antigen CD307c) |
| MPZL1 PZR | Myelin protein zero-like protein 1 (Protein zero-related) |
| UNQ849/PRO1787 | |
| PILRA | Paired immunoglobulin-like type 2 receptor alpha (Cell surface |
| | receptor FDF03) (Inhibitory receptor PILR-alpha) |
| PVR PVS | Poliovirus receptor (Nectin-like protein 5) (NECL-5) (CD antigen CD155) |
| SIGLEC11 | Sialic acid-binding Ig-like lectin 11 (Sialic acid-binding lectin 11) (Siglec- |
| UNQ9222/PRO28718 | 11) |
| SIGLEC10 SLG2 | Sialic acid-binding Ig-like lectin 10 (Siglec-10) (Siglec-like protein 2) |
| UNQ477/PRO940 | |
| TNFRSF10D DCR2 TRAILR4 | Tumor necrosis factor receptor superfamily member 10D (Decoy |
| TRUNDD UNQ251/PRO288 | receptor 2) (DcR2) (TNF-related apoptosis-inducing ligand receptor 4) |
| | (TRAIL receptor 4) (TRAIL-R4) (TRAIL receptor with a truncated death |
| | domain) (CD antigen CD264) |
| TNFRSF10A APO2 DR4 | Tumor necrosis factor receptor superfamily member 10A (Death |
| TRAILR1 | receptor 4) (TNF-related apoptosis-inducing ligand receptor 1) (TRAIL |
| | receptor 1) (TRAIL-R1) (CD antigen CD261) |
| TNFRSF10B DR5 KILLER | Tumor necrosis factor receptor superfamily member 10B (Death |
| TRAILR2 TRICK2 ZTNFR9 | receptor 5) (TNF-related apoptosis-inducing ligand receptor 2) (TRAIL |
| UNQ160/PRO186 | receptor 2) (TRAIL-R2) (CD antigen CD262) |
| CD200R1 CD200R CRTR2 | Cell surface glycoprotein CD200 receptor 1 (CD200 cell surface |
| MOX2R OX2R | glycoprotein receptor) (Cell surface glycoprotein OX2 receptor 1) |
| UNQ2522/PRO6015 | |
| CD″XX″ | cluster of differentiation |
| RARRES1 | Retinoic Acid Receptor Responder (Tazarotene Induced) 1) |
| CCKBR | Cholecystokinin B Receptor |
| GALR1 | galanin receptor 1 |
| CUBN | Cubilin |
| MUC1 | Mucin 1 |
| 5T4 | Trophoblast glycoprotein, also known as TPBG |
| ROR1 | orphan-receptor tyrosine-kinase-like surface |
| Nkp30 | Natural cytotoxicity receptors (Synonym CD337) |
| NKG2D | Killer cell lectin-like receptor subfamily K, member 1 |
| CS1 | SLAM family member 7 |
| MART1 | Antigen LB39-AA, called also Antigen SK29-AA |
| WT1 | Wilms tumor protein |
| LMP2 | latent membrane protein 2 |
| gp100 | Glycoprotein 100 or Melanocyte protein PMEL |
| bcr-abl | called also BCR/ABL fusion protein isoform X8 |
| hTERT | Telomerase transcriptase |
| EphA2 | Eph receptor A2 |
| ERG | |
| PAX3 | Paired box protein Pax-3 |
| PD-1 | Programmed cell death protein 14-1BB |
| OX40 | tumor necrosis factor ligand superfamily member 4 |
| PSMA | Prostate specific membrane antigen |
| ICOS | Inducible T-cell costimulator |
| CTLA-4 | Cytotoxic T-lymphocyte protein 4 |
| LAG3 | Lymphocyte activation gene 3 protein |
| 2B4: | Natural killer cell receptor 2B4 (CD244 |
| CTLA-4 | Cytotoxic T-lymphocyte protein 4 |
| TIM-3 | Protein timeless |
| TIGIT | Protein Tigit |
| SIRPA | Tyrosine-protein phosphatase non-receptor type substrate 1 |
| ALK | ALK tyrosine kinase |
| Endoglin | also called CD105 |
| PD-L1 | Programmed cell death 1 ligand 1 |
| PD-L2 | Programmed cell death 1 ligand 2 |
| ICAM | Intercellular adhesion molecule |
| TCR | T cell receptor |
| Cas9 | CRISPR associated protein 9 |

The present invention provides an inhibitory chimeric antigen receptor (N-CAR) comprising:
an extracellular domain comprising an antigen binding domain;
a transmembrane domain;
an intracellular domain;

wherein said N-CAR comprises a polypeptide sequence involved in inducing an inhibitory transduction signal,

said polypeptide sequence comprises at least one sequence from a Tumor-necrosis-factor related apoptosis inducing ligand (TRAIL) receptor or at least one sequence from a CD200 receptor 1.

The present invention provides an inhibitory chimeric antigen receptor (N-CAR) comprising:
an extracellular domain comprising an antigen binding domain;
a transmembrane domain;
an intracellular domain;

wherein said N-CAR comprises a polypeptide sequence involved in inducing an inhibitory transduction signal upon binding of said extracellular domain comprising an antigen binding domain to an antigen and/or resulting in a decrease in CTL activity,

said polypeptide sequence comprises at least one sequence from a Tumor-necrosis-factor related apoptosis inducing ligand (TRAIL) receptor or at least one sequence from a CD200 receptor 1.

The present invention provides a N-CAR according to the above, comprising a polypeptide sequence from a Tumor-necrosis-factor related apoptosis inducing ligand (TRAIL) receptor.

The present invention provides a N-CAR according to the above comprising at least one polypeptide sequence from a polypeptide sequence selected from the list consisting of SEQ ID NO: 33 (human TR10D), SEQ ID NO: 34 (human TR10A) or SEQ ID NO: 35 (human TR10B) and a fragment thereof.

The present invention provides a N-CAR according to any one of the above embodiments, wherein said polypeptide sequence has more than 80%, preferably 90% and more preferably 95% identity with a sequence from SEQ ID NO: 33, SEQ ID NO: 34 or SEQ ID NO: 35 or a fragment thereof.

The present invention provides a N-CAR according to any one of the above embodiments, comprising at least one of the following polypeptide sequences: amino acids No 181-386 from SEQ ID NO: 33 (human TR10D), amino acids No 230-468 from SEQ ID NO: 34 (human TR10A) or of amino acids N° 179-440 from SEQ ID NO: 35 (human TR10B), or a fragment thereof.

The present invention provides a N-CAR according to any one of the above embodiments wherein said antigen binding domain binds to a cell surface antigen N, and N being not expressed on a cancerous cell and N being expressed on a non-cancerous cell or a healthy cell.

The present invention provides a N-CAR according to any one of the above embodiments, wherein the antigen binding domain binds to a cell-surface antigen N, N being present in normal tissue but not present or present at undetectable level on a tumor as determined by FACS or western blot analysis or by any appropriate technique allowing proteins to be quantified.

In the present invention non-cancerous cell or a healthy cell also expressing a P antigen, said

P antigen being also expressed or over expressed on a cancerous cell.

The present invention provides a N-CAR according to any one of the above embodiments wherein said antigen binding domain binds to at least one cell surface antigen N selected from CD56, CD205, CD83, CD206, CD200, CD36, troponin C, beta-1 integrin, CCKBR, GALR1 CUBN, CD4, CD20, CD22, CD25, MUC1, CD19, BCMA, and PSMA.

The present invention also provides

The N-CAR according to the above embodiments comprising at least one polypeptide sequence consisting essentially of amino acids No 181-386 from SEQ ID NO: 33 (human TR10D).

The N-CAR according to any one of the above embodiments, comprising at least one polypeptide sequence consisting essentially of amino acids No 230-468 from SEQ ID NO: 34 (human TR10A).

The N-CAR according to any one of the above embodiments, comprising at least one polypeptide sequence consisting essentially of amino acids No 179-440 from SEQ ID NO: 35 (human TR10B).

The N-CAR according comprising at least one polypeptide sequence from a CD200 Receptor 1, preferably comprising a sequence of SEQ ID NO. 36 or a fragment thereof.

An N-CAR according to any one of the above embodiments, wherein said N-CAR is a single chain (sc) N CAR or a multi-chain (mc) N-CAR.

The N-CAR according to any one of the above embodiments comprising at least one polypeptide sequence encoded by a sequence selected from the list consisting in SEQ ID NO. 102 to SEQ ID NO. 212.

The N-CAR according to any one of the above embodiments wherein the antigen binding domain binds to an off-tissue antigen.

An N-CAR according to the above embodiments, wherein said extracellular domain comprises at least one a single chain variable fragment scFv.

The N-CAR according to any one of the above embodiments, wherein said antigen binding domain comprises a Fv, a Fab, or a (Fab′)2.

The N-CAR according to any one of the above embodiments wherein said antigen binding domain binds to a cell surface antigen N, expressed on a non cancerous cell or a healthy cell expressing a P antigen.

In one embodiment N is P, in a preferred embodiment N is not P.

The N-CAR according to any one of above embodiments, wherein said antigen binding domain binds to CD19, CD20, CD22, BCMA, PSMA, CD56, CD205, CD83, CD206, CD200 or CD36.

The N-CAR according to any one of above embodiments, wherein said antigen binding domain binds to troponin C, beta-1 integrin, CCKBR, GALR1 or CUBN.

The N-CAR according to any one of above embodiments, wherein said antigen binding domain binds to CD4, CD20, CD22, CD25 or MUC1.

The N-CAR according to any one of above embodiments, wherein said antigen binding domain binds to troponin C, beta-1 integrin, CCKBR, GALR1 or MUC1.

The N-CAR according to any one of above embodiments wherein the transmembrane domain comprises the transmembrane region of PD-1.

The N-CAR according to any one of above embodiments wherein the N-CAR comprises the transmembrane region of PD-1, or a fragment thereof.

The N-CAR according to any one of above embodiments wherein the transmembrane domain comprises the transmembrane region(s) of CD8 alpha.

The N-CAR according to any one of the above embodiments wherein the transmembrane domain is attached to the extracellular domain of the N-CAR via a hinge.

The N-CAR according to the above embodiments wherein the hinge is a human immunoglobulin hinge.

The N-CAR according to the above embodiments wherein the hinge is an IgG1 hinge or a CD8 alpha hinge.

The N-CAR according to any one of the above embodiments wherein the transmembrane domain comprises a transmembrane region(s) of the alpha, beta or zeta chain of the T-cell receptor, PD-1, 4-1BB, OX40, ICOS, CTLA-4, LAG3, 2B4, BTLA4, TIM-3, TIGIT, SIRPA, CD28, CD3 epsilon, CD45, CD4, CD5, CD8, CD9, CD16, CD22, CD33, CD37, CD64, CD80, CD86, CD134, CD137 or CD154.

The present invention provides a vector encoding a N-CAR according to any one of the above embodiments.

The present invention provides an immune cell, preferably a primary immune T cell comprising a P-CAR comprising:
an extracellular domain comprising an antigen binding domain;
a transmembrane domain;
an intracellular domain; preferably a intracellular domain comprising an activator transducing domain and an co-stimulatory domain,

and a N-CAR according to any one of the above embodiments.

In a preferred embodiment activation of the N-CAR inhibits the signal transduction activity related to the P-CAR, resulting in particular in a decrease in the CTL activity or the immune cell bearing a N-CAR and a P-CAR.

In one preferred embodiment the present invention provides a immune cell according to the above embodiment wherein at least one gene encoding a TCR alpha or a TCR beta subunit is inactivated, preferably by deletion using a specific endonuclease.

In one embodiment the present invention provides an immune cell according to any one of the above embodiments wherein at least one gene encoding a TCR and a gene encoding a deoxicitidine kinase (dck) are inactivated, preferably by deletion using a endonuclease, preferably a TALEN.

In one embodiment the present invention provides an immune cell according to any one of the above embodiments for use as a medicament.

In one embodiment the present invention provides an immune cell according to any one of the above embodiments for use in the prevention or treatment of a haematological cancer condition, preferably a relapsed refractory haematological cancer.

The immune cell according to the above embodiments, wherein said haematological cancer condition is leukemia or myeloma, preferably relapsed and/or refractory leukemia or relapsed and/or refractory myeloma.

In one embodiment the present invention provides a method of engineering an immune cell according to any one of the above embodiments comprising:
(a) Providing an immune cell; optionally deleting a candidate gene, said candidate gene being preferably TCRA and dCK
(b) Expressing a N-CAR and a P-CAR according to the invention at the cell surface.
(c) optionally deleting a candidate gene, said candidate gene being preferably selected from TCRA, PD1, CTLA4 and dCK

In one embodiment the present invention provides a method as above wherein said immune cells are provided from a donor, preferably a healthy donor.

In one embodiment the present invention provides a vector comprising a sequence selected from the list consisting in SEQ ID NO. 102 to SEQ ID NO. 212.

The immune cell according to the above embodiments is provided wherein the immune cell is a T-cell, preferably a CD4 T cell or a CD8 T cell, more preferably a primary CD8 T cell.

The immune cell according to the above embodiments wherein the T-cell is a human T-cell, preferably a primary immune T cell.

The immune cell according to the above embodiments wherein the P CAR binds to at least one of the following antigen CD123, CD22, CS1, CD38, CD19, CD33, CD20.

The immune cell according to the above embodiments is selected from primary inflammatory T-lymphocytes, primary cytotoxic T-lymphocytes, primary regulatory T-lymphocytes or primary helper T-lymphocytes.

In one embodiment the present invention provides an immune cell according to any one of the above embodiments, wherein the cell surface antigen to which the antigen binding domain of the P-CAR binds to is CD38 and the cell surface antigen to which the antigen binding domain of the N-CAR binds to is CD56, CD205, CD83, CD206, CD200 or CD36.

In one embodiment the present invention provides an immune cell according to any one of the above embodiments, wherein the cell surface antigen to which the antigen binding domain of the P-CAR binds to is CS1 and the cell surface antigen to which the antigen binding domain of the N-CAR binds to is troponin C, beta-1 integrin, CCKBR, GALR1 or CUBN.

In one embodiment the present invention provides an immune cell according to any one of the above embodiments, wherein the cell surface antigen to which the antigen binding domain of the P-CAR binds to is CD123 and the antigen to which the antigen binding domain of the N-CAR binds is CD4, CD20, CD22, CD25 or MUC1.

In one embodiment the present invention provides an immune cell according to any one of the above embodiments wherein the cell surface antigen to which the antigen binding domain of the P-CAR binds to is ROR1 and the cell surface antigen to which the antigen binding domain of the N-CAR binds is troponin C, beta-1 integrin, CCKBR, GALR1 or MUC1.

In one embodiment the present invention provides an immune cell for use as a medicament

In one embodiment the present invention provides an immune cell for the treatment of a leukemia selected from the group consisting of acute myelogenous leukemia (AML).

The present invention provides an immune cell according to the above embodiments for use in therapy, wherein the condition is a pre-malignant or malignant cancer condition characterized by CD123-expressing cells or by CLL-1 expressing cells

The immune cell according to any one of the above embodiments, wherein said haematological cancer condition is multiple myeloma (MM).

The immune cell according to the above embodiments for use in therapy, wherein the condition is a pre-malignant or malignant cancer condition characterized by CD38-expressing cells.

The immune cell according to the above embodiments, wherein said haematological cancer condition is chronic lymphocytic leukemia (CLL).

The immune cell according to the above embodiments for use in therapy, wherein the condition is a pre-malignant or malignant cancer condition characterized by CS1-expressing cells or by ROR1-expressing cells.

The immune cell according to the above embodiments for use in therapy, wherein the condition is a solid tumor such as breast, colon, lung, or kidney tumor characterized especially by ROR1-expressing cells.

The immune cell according to the above embodiments for use in therapy, wherein the condition is a pre-malignant or malignant cancer condition characterized by CD22-expressing cells.

The immune cell according to any one of the above wherein the reduction of activation of the immune cells when both the P-CAR and N-CAR bind to their respective antigens is increased, preferably by at least 5%, 10%, 15%, 20% or 30% as compared to the same immune cell wherein a P-CAR alone binds to its cell surface antigen, and/or as compared to an immune cell expressing a full intracellular domain of PD-1 or a full intracellular domain of CTLA-4 as an intracellular domain of said N-CAR.

The immune cell according to any one of the above embodiments wherein the level of activation of the immune cell is determined by measuring cytokine production.

The immune cell according to claim 34 wherein the cytokine is IFNgamma or TNFalpha.

The immune cell according to any one of the above embodiments wherein the cytokine production is measured by ELISA and/or FACS and/or luminex.

The immune cell according to any one of the above embodiments wherein the level of activation of the immune cell is determined by the level of degranulation.

The immune cell according to any one of the above embodiments wherein degranulation is measured by measuring expression of CD107a by FACS.

The immune cell according to any one of the above embodiments wherein the level of activation of the immune cell is measured by monitoring the ability of the immune cell to kill target cells.

The present invention provides a method of engineering an immune cell according to any one of the above embodiments comprising:
(a) Introducing into said cell at least one polynucleotide encoding the N-CAR and at least one polynucleotide encoding the CAR;
(b) Expressing said polynucleotides into said cell.

The present invention provides a method for treating a patient in need thereof comprising:
a) Providing an immune cell according to any one of the above embodiments, and;
b) Administrating said T-cells to said patient.

The present invention discloses a method for treating a patient according to any one of the above embodiments wherein said immune cells are recovered from patients.

The different objects of the present invention are disclosed in details as follows

Inhibitory Chimeric Antigen Receptor (iCAR or N-CAR)

The present invention relates to an inhibitory chimeric antigen receptor (iCAR or N-CAR) comprising:
an extracellular domain comprising an antigen binding domain;
a transmembrane domain;
an intracellular domain;

wherein said intracellular domain comprises a polypeptide sequence involved in transduction signal said polypeptide sequence is not significantly expressed in T-cell, preferably in primary T cells, and/or said polypeptide sequence is from a Tumor-necrosis-factor related apoptosis inducing ligand (TRAIL) receptor and/or CD200 receptor 1,

provided said polypeptide sequence not expressed in T cells is not an ITMS, preferably it does not consists in (or does not comprise) a sequence chosen in a group consisting of SEQ ID NO:13 (human SIGL8), SEQ ID NO:14 (human SIGL7), SEQ ID NO:17 (human SIGL5), SEQ ID NO:20 (human SIGL9), SEQ ID NO: 21 (human SIGL6), SEQ ID NO:22 (human CD33), SEQ ID NO:26 (human SIG12), SEQ ID NO:31 (human SIG11), SEQ ID NO:32 (human SIG10) and SEQ ID NO:19 (human PECA1).

Intracellular Domain of the CAR-N According to the Invention

According to one embodiment, the intracellular domain of the N-CAR comprises a polypeptide sequence from a receptor involved in transduction signal which is not significantly expressed in non-engineered T-cells. By “not significantly expressed” is meant that the protein involved in the transduction signal, which intracellular domain is from, is not expressed or expressed at a significant lower level in the same culture or growth conditions in a non-engineered T-Cell. By “engineered T-cells” are meant T-cells that have been genetically modified to express or to unable expression of a given genetic sequence.

Here, by “not significantly expressed”, it is meant that the expression of said polypeptide, preferably cell surface expression below the level of detection using any appropriate technique such as flow cytometry analysis, western blot or Elisa test or that said polypeptide is expressed at a level of less than 20% and preferably less than 10% and more preferably at undetectable level in a given cell as compared the expression of said polypeptide measured in a cell known to express said polypeptide used as a positive control.

This can be tested by currently used techniques allowing expression of proteins to be measured and quantified that is using western blot, flow cytometry analysis, Elisa test and others.

According to one embodiment, the intracellular domain of the N-CAR comprises a polypeptide sequence involved in transduction signal of a receptor, and said receptor is not significantly expressed in T-cells.

In a preferred embodiment, said polypeptide sequence is from a sequence selected from the group consisting of SEQ ID NO: 1 (human KI2L2), SEQ ID NO: 2 (human KI2L1), SEQ ID NO:3 human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA), SEQ ID NO:30 (human PVR), and a fragment thereof.

In a more preferred embodiment said polypeptide sequences is SEQ ID NO: 1 (human KI2L2) or a fragment thereof.

In a more preferred embodiment said polypeptide sequences is SEQ ID NO: 2 (human KI2L1), or a fragment thereof.

In a more preferred embodiment said polypeptide sequences is SEQ ID NO:3 (human FCG2B), or a fragment thereof.

In a more preferred embodiment said polypeptide sequences is SEQ ID NO:4 (human KI2L3), or a fragment thereof.

In a more preferred embodiment said polypeptide sequences is SEQ ID NO: 5 (human KI3L2), or a fragment thereof.

In a more preferred embodiment said polypeptide sequences is SEQ ID NO:6 (human KI2L4), or a fragment thereof.

In a more preferred embodiment said polypeptide sequences is SEQ ID NO:7 (human KI3L1), or a fragment thereof.

In a more preferred embodiment said polypeptide sequences is SEQ ID NO:8 (human KI2LA), or a fragment thereof.

In a more preferred embodiment said polypeptide sequences is SEQ ID NO:9 (human MILR1), or a fragment thereof.

In a more preferred embodiment said polypeptide sequences is SEQ ID NO:10 (human LIRB4), or a fragment thereof.

In a more preferred embodiment said polypeptide sequences is SEQ ID NO:11 (human LIRB3), or a fragment thereof.

In a more preferred embodiment said polypeptide sequences is SEQ ID NO:12 (human KI3L3), or a fragment thereof.

In a more preferred embodiment said polypeptide sequences is SEQ ID NO:15 (human LIRB5), or a fragment thereof.

In a more preferred embodiment said polypeptide sequences is SEQ ID NO:16 (human LIRB2), or a fragment thereof.

In a more preferred embodiment said polypeptide sequences is SEQ ID NO:18 (human FCRL4), or a fragment thereof.

In a more preferred embodiment said polypeptide sequences is SEQ ID NO:23 (human FCRL5), or a fragment thereof.

In a more preferred embodiment said polypeptide sequences is SEQ ID NO:24 (human FCRL2), or a fragment thereof.

In a more preferred embodiment said polypeptide sequences is SEQ ID NO: 25 (human FCRL1), or a fragment thereof.

In a more preferred embodiment said polypeptide sequences is SEQ ID NO:27 (human FCRL3), or a fragment thereof.

In a more preferred embodiment said polypeptide sequences is SEQ ID NO:28 (human MPZL1), or a fragment thereof.

In a more preferred embodiment said polypeptide sequences is SEQ ID NO:29 (human PILRA), or a fragment thereof.

In a more preferred embodiment said polypeptide sequences is SEQ ID NO:30 (human PVR), or a fragment thereof.

In another embodiment according to the invention, said polypeptide sequence has more than 80%, preferably 90% and more preferably 95% identity with a sequence from a sequence selected from SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA), SEQ ID NO:30 (human PVR).

The above sequences are presented in the following Table 1.

The architecture of some exemplary N-CARs is presented in the following Table 2.

TABLE 1
Amino-acid sequences of intracellular domain from a receptor involved in transduction
signal which is not significantly expressed in non-engineered T-cell (Sequences SEQ NO: 13
(human SIGL8), SEQ ID NO: 14 (human SIGL7), SEQ ID NO: 17 (human SIGL5), SEQ ID NO: 20
(human SIGL9), SEQ ID NO: 21 (human SIGL6), SEQ ID NO: 22 (human CD33), SEQ ID NO: 26
(human SIG12), SEQ ID NO: 31 (human SIG11), SEQ ID NO: 32 (human SIG10) and SEQ ID NO: 19
being not part of the present invention)

| | UniProt | SEQ | |
|---|---|---|---|
| Name | ID | ID NO: | |
| human KI2L2 | P43627 | 1 | MWSHLNRLLFWSIFSSVTCRKAVLDCEAMKTNEFPSPCLDSKTKVVMKGQNVSMFCSHKNKSLQITYSLFRRK |
| | | | THLGTQDGKGEPAIFNLSITEAHESGPYKCKAQVTSCSKYSRDFSFTIVDPVTSPVLNIMVIQTETDRHITLH |
| | | | CLSVNGSLPINYTFFENHVAISPAISKYDREPAEFNLTKKNPGEEEEYRCEAKNRLPNYATYSHPVTMPSTGG |
| | | | DSCPFCLKLLLPGLLLLLVVIILILAFWVLPKYKTRKAMRNNVPRDRGDTAMEVGIYANILEKQAKEESVPEV |
| | | | GSRPCVSTAQDEAKHSQELQYATPVFQEVAPREQEACDSYKSGYVYSELNF |
| human KI2L1 | P43626 | 2 | MIPTFTALLCLGLSLGPRTHMQAGPLPKPTLWAEPGSVISWGNSVTIWCQGTLEAREYRLDKEESPAPWDRQN |
| | | | PLEPKNKARFSIPSMTEDYAGRYRCYYRSPVGWSQPSDPLELVMTGAYSKPTLSALPSPLVTSGKSVTLLCQS |
| | | | RSPMDTFLLIKERAAHPLLHLRSEHGAQQHQAEFPMSPVTSVHGGTYRCFSSHGFSHYLLSHPSDPLELIVSG |
| | | | SLEDPRPSPTRSVSTAAGPEDQPLMPTGSVPHSGLRRHWEVLIGVLVVSILLLSLLLFLLLQHWRQGKHRTLA |
| | | | QRQADFQRPPGAAEPEPKDGGLQRRSSPAADVQGENFCAAVKNTQPEDGVEMDTRQSPHDEDPQAVTYAKVKH |
| | | | SRPRREMASPPSPLSGEFLDTKDRQAEEDRQMDTEAAASEAPQDVTYAQLHSFTLRQKATEPPPSQEGASPAE |
| | | | PSVYATLAIH |
| human FCG2B | P31994 | 3 | MTPALTALLCLGLSLGPRTRVQAGPFPKPTLWAEPGSVISWGSPVTIWCQGSQEAQEYRLHKEGSPEPLDRNN |
| | | | PLEPKNKARFSIPSMTEHHAGRYRCHYYSSAGWSEPSDPLEMVMTGAYSKPTLSALPSPVVASGGNMTLRCGS |
| | | | QKGYHHFVLMKEGEHQLPRTLDSQQLHSRGFQALFPVGPVTPSHRWRFTCYYYYTNTPWVWSHPSDPLEILPS |
| | | | GVSRKPSLLTLQGPVLAPGQSLTLQCGSDVGYNRFVLYKEGERDFLQRPGQQPQAGLSQANFTLGPVSPSNGG |
| | | | QYRCYGAHNLSSEWSAPSDPLNILMAGQIYDTVSLSAQPGPTVASGENVTLLCQSWWQFDTFLLTKEGAAHPP |
| | | | LRLRSMYGAHKYQAEFPMSPVTSAHAGTYRCYGSYSSNPHLLSHPSEPLELVVSGHSGGSSLPPTGPPSTPGL |
| | | | GRYLEVLIGVSVAFVLLLFLLLFLLLRRQRHSKHRTSDQRKTDFQRPAGAAETEPKDRGLLRRSSPAADVQEE |
| | | | NLYAAVKDTQSEDRVELDSQSPHDEDPQAVTYAPVKHSSPRREMASPPSSLSGEFLDTKDRQVEEDRQMDTEA |
| | | | AASEASQDVTYAQLHSLTLRRKATEPPPSQEGEPPAEPSIYATLAIH |
| human KI2L3 | P43628 | 4 | MSLMVVSMACVGFFLLEGPWPHVGGQDKPFLSAWPGTVVSEGQHVTLQCRSRLGFNEFSLSKEDGMPVPELYN |
| | | | RIFRNSFLMGPVTPAHAGTYRCCSSHPHSPTGWSAPSNPVVIMVTGVHRKPSLLAHPGPLVKSGETVILQCWS |
| | | | DVRFERFLLHREGITEDPLRLVGQLHDAGSQVNYSMGPMTPALAGTYRCFGSVTHLPYELSAPSDPLDIVVVG |
| | | | LYGKPSLSAQPGPTVQAGENVTLSCSSRSLFDIYHLSREAEAGELRLTAVLRVNGTFQANFPLGPVTHGGNYR |
| | | | CFGSFRALPHAWSDPSDPLPVSVTGNSRHLHVLIGTSVVIIPFAILLFFLLHRWCANKKNAVVMDQEPAGNRT |
| | | | VNREDSDEQDPQEVTYAQLNHCVFTQRKITRPSQRPKTPPTDTSV |
| human KI3L2 | P43630 | 5 | MLLLLLLLPLLWGTKGMEGDRQYGDGYLLQVQELVTVQEGLCVHVPCSFSYPQDGWTDSDPVHGYWFRAGDRP |
| | | | YQDAPVATNNPDREVQAETQGRFQLLGDIWSNDCSLSIRDARKRDKGSYFFRLERGSMKWSYKSQLNYKTKQL |
| | | | SVFVTALTHRPDILILGTLESGHSRNLTCSVPWACKQGTPPMISWIGASVSSPGPTTARSSVLTLTPKPQDHG |
| | | | TSLTCQVTLPGTGVTTTSTVRLDVSYPPWNLTMTVFQGDATASTALGNGSSLSVLEGQSLRLVCAVNSNPPAR |
| | | | LSWTRGSLTLCPSRSSNPGLLELPRVHVRDEGEFTCRAQNAQGSQHISLSLSLQNEGTGTSRPVSQVTLAAVG |
| | | | GAGATALAFLSFCIIFIIVRSCRKKSARPAAGVGDTGMEDAKAIRGSASQGPLTESWKDGNPLKKPPPAVAPS |
| | | | SGEEGELHYATLSFHKVKPQDPQGQEATDSEYSEIKIHKRETAETQACLRNHNPSSKEVRG |
| human KI2L4 | Q99706 | 6 | MLLLLLLPLLWGRERVEGQKSNRKDYSLTMQSSVTVQEGMCVHVRCSFSYPVDSQTDSDPVHGYWFRAGNDIS |
| | | | WKAPVATNNPAWAVQEETRDRFHLLGDPQTKNCTLSIRDARMSDAGRYFFRMEKGNIKWNYKYDQLSVNVTAL |
| | | | THRPNILIPGTLESGCFQNLTCSVPWACEQGTPPMISWMGTSVSPLHPSTTRSSVLTLIPQPQHHGTSLTCQV |
| | | | TLPGAGVTTNRTIQLNVSYPPQNLTVTVFQGEGTASTALGNSSSLSVLEGQSLRLVCAVDSNPPARLSWTWRS |
| | | | LTLYPSQPSNPLVLELQVHLGDEGEFTCRAQNSLGSQHVSLNLSLQQEYTGKMRPVSGVLLGAVGGAGATALV |
| | | | FLSFCVIFIVVRSCRKKSARPAADVGDIGMKDANTIRGSASQGNLTESWADDNPRHHGLAAHSSGEEREIQYA |
| | | | PLSFHKGEPQDLSGQEATNNEYSEIKIPK |
| human KI3L1 | P43629 | 7 | MTLTLSVLICLGLSVGPRTCVQAGTLPKPTLWAEPASVIARGKPVTLWCQGPLETEEYRLDKEGLPWARKRQN |
| | | | PLEPGAKAKFHIPSTVYDSAGRYRCYYETPAGWSEPSDPLELVATGFYAEPTLLALPSPVVASGGNVTLQCDT |
| | | | LDGLLTFVLVEEEQKLPRTLYSQKLPKGPSQALFPVGPVTPSCRWRFRCYYYYRKNPQVWSNPSDLLEILVPG |
| | | | VSRKPSLLIPQGSVVARGGSLTLQCRSDVGYDIFVLYKEGEHDLVQGSGQQPQAGLSQANFTLGPVSRSHGGQ |
| | | | YRCYGAHNLSPRWSAPSDPLDILIAGLIPDIPALSVQPGPKVASGENVTLLCQSWHQIDTFFLTKEGAAHPPL |
| | | | CLKSKYQSYRHQAEFSMSPVTSAQGGTYRCYSAIRSYPYLLSSPSYPQELVVSGPSGDPSLSPTGSTPTPGPE |
| | | | DQPLTPTGLDPQSGLGRHLGVVTGVSVAFVLLLFLLLFLLLRHRHQSKHRTSAHFYRPAGAAGPEPKDQGLQK |
| | | | RASPVADIQEEILNAAVKDTQPKDGVEMDARAAASEAPQDVTYAQLHSLTLRREATEPPPSQEREPPAEPSIY |
| | | | APLAIH |
| human KI2LA | Q8N109 | 8 | MTPIVTVLICLGLSLGPRTHVQTGTIPKPTLWAEPDSVITQGSPVTLSCQGSLEAQEYRLYREKKSASWITRI |
| | | | RPELVKNGQFHIPSITWEHTGRYGCQYYSRARWSELSDPLVLVMTGAYPKPTLSAQPSPVVTSGGRVTLQCES |
| | | | QVAFGGFILCKEGEEEHPQCLNSQPHARGSSRAIFSVGPVSPNRRWSHRCYGYDLNSPYVWSSPSDLLELLVP |
| | | | GVSKKPSLSVQPGPVVAPGESLTLQCVSDVGYDRFVLYKEGERDLRQLPGRQPQAGLSQANFTLGPVSRSYGG |
| | | | QYRCYGAHNLSSECSAPSDPLDILITGQIRGTPFISVQPGPTVASGENVTLLCQSWRQFHTFLLTKAGAADAP |
| | | | LRLRSIHEYPKYQAEFPMSPVTSAHAGTYRCYGSLNSDPYLLSHPSEPLELVVSGPSMGSSPPPTGPISTPAG |
| | | | PEDQPLTPTGSDPQSGLGRHLGVVIGILVAVVLLLLLLLLLFLILRHRRQGKHWTSTQRKADFQHPAGAVGPE |
| | | | PTDRGLQWRSSPAADAQEENLYAAVKDTQPEDGVEMDTRAAASEAPQDVTYAQLHSLTLRRKATEPPPSQERE |
| | | | PPAEPSIYATLAIH |
| human MILR1 | Q7Z6M3 | 9 | MLPLLLLPLLWGGSLQEKPVYELQVQKSVTVQEGLCVLVPCSFSYPWRSWYSSPPLYVYWFRDGEIPYYAEVV |
| | | | ATNNPDRRVKPETQGRFRLLGDVQKKNCSLSIGDARMEDTGSYFFRVERGRDVKYSYQQNKLNLEVTALIEKP |
| | | | DIHFLEPLESGRPTRLSCSLPGSCEAGPPLTFSWTGNALSPLDPETTRSSELTLTPRPEDHGTNLTCQMKRQG |
| | | | AQVTTERTVQLNVSYAPQTITIFRNGIALEILQNTSYLPVLEGQALRLLCDAPSNPPAHLSWFQGSPALNATP |
| | | | ISNTGILELRRVRSAEEGGFTCRAQHPLGFLQIFLNLSVYSLPQLLGPSCSWEAEGLHCRCSFRARPAPSLCW |
| | | | RLEEKPLEGNSSQGSFKVNSSSAGPWANSSLILHGGLSSDLKVSCKAWNIYGSQSGSVLLLQGRSNLGTGVVP |
| | | | AALGGAGVMALLCICLCLIFFLIVKARRKQAAGRPEKMDDEDPIMGTITSGSRKKPWPDSPGDQASPPGDAPP |
| | | | LEEQKELHYASLSFSEMKSREPKDQEAPSTTEYSEIKTSK |
| human LIRB4 | Q8NHJ6 | 10 | MLLWASLLAFAPVCGQSAAAHKPVISVHPPWTTFFKGERVTLTCNGFQFYATEKTTWYHRHYWGEKLTLTPGN |
| | | | TLEVRESGLYRCQARGSPRSNPVRLLFSSDSLILQAPYSVFEGDTLVLRCHRRRKEKLTAVKYTWNGNILSIS |
| | | | NKSWDLLIPQASSNNNGNYRCIGYGDENDVFRSNFKIIKIQELFPHPELKATDSQPTEGNSVNLSCETQLPPE |
| | | | RSDTPLHFNFFRDGEVILSDWSTYPELQLPTVWRENSGSYWCGAETVRGNIHKHSPSLQIHVQRIPVSGVLLE |
| | | | TQPSGGQAVEGEMLVLVCSVAEGTGDTTFSWHREDMQESLGRKTQRSLRAELELPAIRQSHAGGYYCTADNSY |
| | | | GPVQSMVLNVTVRETPGNRDGLVAAGATGGLLSALLLAVALLFHCWRRRKSGVGFLGDETRLPPAPGPGESSH |
| | | | SICPAQVELQSLYVDVHPKKGDLVYSEIQTTQLGEEEEANTSRTLLEDKDVSVVYSEVKTQHPDNSAGKISSK |
| | | | DEES |
| human LIRB3 | O75022 | 11 | MQPRWAQGATMWLGVLLTLLLCSSLEGQENSFTINSVDMKSLPDWTVQNGKNLTLQCFADVSTTSHVKPQHQM |
| | | | LFYKDDVLFYNISSMKSTESYFIPEVRIYDSGTYKCTVIVNNKEKTTAEYQLLVEGVPSPRVTLDKKEAIQGG |
| | | | IVRVNCSVPEEKAPIHFTIEKLELNEKMVKLKREKNSRDQNFVILEFPVEEQDRVLSFRCQARIISGIHMQTS |
| | | | ESTKSELVTVTESFSTPKFHISPTGMIMEGAQLHIKCTIQVTHLAQEFPEIIIQKDKAIVAHNRHGNKAVYSV |
| | | | MAMVEHSGNYTCKVESSRISKVSSIVVNITELFSKPELESSFTHLDQGERLNLSCSIPGAPPANFTIQKEDTI |
| | | | VSQTQDFTKIASKSDSGTYICTAGIDKVVKKSNTVQIVVCEMLSQPRISYDAQFEVIKGQTIEVRCESISGTL |
| | | | PISYQLLKTSKVLENSTKNSNDPAVFKDNPTEDVEYQCVADNCHSHAKMLSEVLRVKVIAPVDEVQISILSSK |
| | | | VVESGEDIVLQCAVNEGSGPITYKFYREKEGKPFYQMTSNATQAFWTKQKASKEQEGEYYCTAFNRANHASSV |
| | | | PRSKILIVRVILAPWKKGLIAVVIIGVIIALLIIAAKCYFLRKAKAKQMPVEMSRPAVPLLNSNNEKMSDPNM |
| | | | EANSHYGHNDDVRNHAMKPINDNKEPLNSDVQYTEVQVSSAESHKDLGKKDTETVYSEVRKAVPDAVESRYSR |
| | | | TEGSLDGT |
| human KI3L3 | Q8N743 | 12 | MLLLLLPLLWGRERAEGQTSKLLTMQSSVTVQEGLCVHVPCSFSYPSHGWIYPGPVVHGYWFREGANTDQDAP |
| | | | VATNNPARAVWEETRDRFHLLGDPHTKNCTLSIRDARRSDAGRYFFRMEKGSIKWNYKHHRLSVNVTALTHRP |
| | | | NILIPGTLESGCPQNLTCSVPWACEQGTPPMISWIGTSVSPLDPSTTRSSVLTLIPQPQDHGTSLTCQVTFPG |
| | | | ASVTTNKTVHLNVSYPPQNLTMTVFQGDGTVSTVLGNGSSLSLPEGQSLRLVCAVDAVDSNPPARLSLSWRGL |
| | | | TLCPSQPSNPGVLELPWVHLRDAAEFTCRAQNPLGSQQVYLNVSLQSKATSGVTQGVVGGAGATALVFLSFCV |
| | | | IFVVVRSCRKKSARPAAGVGDTGIEDANAVRGSASQGPLTEPWAEDSPPDQPPPASARSSVGEGELQYASLSF |
| | | | QMVKPWDSRGQEATDTEYSEIKIHR |
| human SIGL8 | Q9NYZ4 | 13 | MQGAQEASASEMLPLLLPLLWAGALAQERRFQLEGPESLTVQEGLCVLVPCRLPTTLPASYYGYGYWFLEGAD |
| | | | VPVATNDPDEEVQEETRGRFHLLWDPRRKNCSLSIRDARRRDNAAYFFRLKSKWMKYGYTSSKLSVRVMALTH |
| | | | RPNISIPGTLESGHPSNLTCSVPWVCEQGTPPIFSWMSAAPTSLGPRTTQSSVLTITPRPQDHSTNLTCQVTF |
| | | | PGAGVTMERTIQLNVSYAPQKVAISIFQGNSAAFKILQNTSSLPVLEGQALRLLCDADGNPPAHLSWFQGFPA |
| | | | LNATPISNTGVLELPQVGSAEEGDFTCRAQHPLGSLQISLSLFVHWKPEGRAGGVLGAVWGASITTLVFLCVC |
| | | | FIFRVKTRRKKAAQPVQNTDDVNPVMVSGSRGHQHQFQTGIVSDHPAEAGPISEDEQELHYAVLHFHKVQPQE |
| | | | PKVTDTEYSEIKIHK |
| human SIGL7 | Q9Y286 | 14 | MPLLLLLPLLWAGALAMDPNFWLQVQESVTVQEGLCVLVPCTFFHPIPYYDKNSPVHGYWFREGAIISRDSPV |
| | | | ATNKLDQEVQEETQGRFRLLGDPSRNNCSLSIVDARRRDNGSYFFRMERGSTKYSYKSPQLSVHVTDLTHRPK |
| | | | ILIPGTLEPGHSKNLTCSVSWACEQGTPPIFSWLSAAPTSLGPRTTHSSVLIITPRPQDHGTNLTCQVKFAGA |
| | | | GVTTERTIQLNVTYVPQNPUGIFPGDGSGKQETRAGVVHGAIGGAGVTALLALCLCLIFFIVKTHRRKAARTA |
| | | | VGRNDTHPTTGSASPKHQKKSKLHGPTETSSCSGAAPTVEMDEELHYASLNFHGMNPSKDTSTEYSEVRTQ |
| human LIRB5 | O75023 | 15 | MLLWVILLVLAPVSGQFARTPRPIIFLQPPWTTVFQGERVTLTCKGFRFYSPQKTKWYHRYLGKEILRETPDN |
| | | | ILEVQESGEYRCQAQGSPLSSPVHLDFSSASLILQAPLSVFEGDSVVLRCRAKAEVTLNNTIYKNDNVLAFLN |
| | | | KRTDFHIPHACLKDNGAYRCTGYKESCCPVSSNTVKIQVQEPFTRPVLRASSFQPISGNPVTLTCETQLSLER |
| | | | SDVPLRFRFFRDDQTLGLGWSLSPNFQITAMWSKDSGFYWCKAATMPYSVISDSPRSWIQVQIPASHPVLTLS |
| | | | PEKALNFEGTKVTLHCETQEDSLRTLYRFYHEGVPLRHKSVRCERGASISFSLTTENSGNYYCTADNGLGAKP |
| | | | SKAVSLSVTVPVSHPVLNLSSPEDLIFEGAKVTLHCEAQRGSLPILYQFHHEGAALERRSANSAGGVAISFSL |
| | | | TAEHSGNYYCTADNGFGPQRSKAVSLSVTVPVSHPVLTLSSAEALTFEGATVTLHCEVQRGSPQILYQFYHED |
| | | | MPLWSSSTPSVGRVSFSFSLTEGHSGNYYCTADNGFGPQRSEVVSLFVTVPVSRPILTLRVPRAQAVVGDLLE |
| | | | LHCEAPRGSPPILYWFYHEDVTLGSSSAPSGGEASFNLSLTAEHSGNYSCEANNGLVAQHSDTISLSVIVPVS |
| | | | RPILTFRAPRAQAVVGDLLELHCEALRGSSPILYWFYHEDVTLGKISAPSGGGASFNLSLTTEHSGIYSCEAD |
| | | | NGLEAQRSEMVTLKVAVPVSRPVLTLRAPGTHAAVGDLLELHCEALRGSPLILYRFFHEDVTLGNRSSPSGGA |
| | | | SLNLSLTAEHSGNYSCEADNGLGAQRSETVTLYITGLTANRSGPFATGVAGGLLSIAGLAAGALLLYCWLSRK |
| | | | AGRKPASDPARSPSDSDSQEPTYHNVPAWEELQPVYTNANPRGENVVYSEVRIIQEKKKHAVASDPRHLRNKG |
| | | | SPIIYSEVKVASTPVSGSLFLASSAPHR |
| human LIRB2 | Q8N423 | 16 | MLLWSLLVIFDAVTEQADSLTLVAPSSVFEGDSIVLKCQGEQNWKIQKMAYHKDNKELSVFKKFSDFLIQSAV |
| | | | LSDSGNYFCSTKGQLFLWDKTSNIVKIKVQELFQRPVLTASSFQPIEGGPVSLKCETRLSPQRLDVQLQFCFF |
| | | | RENQVLGSGWSSSPELQISAVWSEDTGSYWCKAETVTHRIRKQSLQSQIHVQRIPISNVSLEIRAPGGQVTEG |
| | | | QKLILLCSVAGGTGNVTFSWYREATGTSMGKKTQRSLSAELEIPAVKESDAGKYYCRADNGHVPIQSKVVNIP |
| | | | VRIPVSRPVLTLRSPGAQAAVGDLLELHCEALRGSPPILYQFYHEDVTLGNSSAPSGGGASFNLSLTAEHSGN |
| | | | YSCEANNGLGAQCSEAVPVSISGPDGYRRDLMTAGVLWGLFGVLGFTGVALLLYALFHKISGESSATNEPRGA |
| | | | SRPNPQEFTYSSPTPDMEELQPVYVNVGSVDVDVVYSQVWSMQQPESSANIRTLLENKDSQVIYSSVKKS |
| human SIGL5 | O15389 | 17 | MLPRLLLLICAPLCEPAELFLIASPSHPTEGSPVTLTCKMPFLQSSDAQFQFCFFRDTRALGPGWSSSPKLQI |
| | | | AAMWKEDTGSYWCEAQTMASKVLRSRRSQINVHRVPVADVSLETQPPGGQVMEGDRLVLICSVAMGTGDITFL |
| | | | WYKGAVGLNLQSKTQRSLTAEYEIPSVRESDAEQYYCVAENGYGPSPSGLVSITVRIPVSRPILMLRAPRAQA |
| | | | AVEDVLELHCEALRGSPPILYWFYHEDITLGSRSAPSGGGASFNLSLTEEHSGNYSCEANNGLGAQRSEAVTL |
| | | | NFTVPTGARSNHLTSGVIEGLLSTLGPATVALLFCYGLKRKIGRRSARDPLRSLPSPLPQEFTYLNSPTPGQL |
| | | | QPIYENVNVVSGDEVYSLAYYNQPEQESVAAETLGTHMEDKVSLDIYSRLRKANITDVDYEDAM |
| human FCRL4 | Q96PJ5 | 18 | MLLLLLLLPPLLCGRVGAKEQKDYLLTMQKSVTVQEGLCVSVLCSFSYPQNGWTASDPVHGYWFRAGDHVSRN |
| | | | IPVATNNPARAVQEETRDRFHLLGDPQNKDCTLSIRDTRESDAGTYVFCVERGNMKWNYKYDQLSVNVTASQD |
| | | | LLSRYRLEVPESVTVQEGLCVSVPCSVLYPHYNWTASSPVYGSWFKEGADIPWDIPVATNTPSGKVQEDTHGR |
| | | | FLLLGDPQTNNCSLSIRDARKGDSGKYYFQVERGSRKWNYIYDKLSVHVTALTHMPTFSIPGTLESGHPRNLT |
| | | | CSVPWACEQGTPPTITWMGASVSSLDPTITRSSMLSLIPQPQDHGTSLTCQVTLPGAGVTMTRAVRLNISYPP |
| | | | QNLTMTVFQGDGTASTTLRNGSALSVLEGQSLHLVCAVDSNPPARLSWTWGSLTLSPSQSSNLGVLELPRVHV |
| | | | KDEGEFTCRAQNPLGSQHISLSLSLQNEYTGKMRPISGVTLGAFGGAGATALVFLYFCIIFVVVRSCRKKSAR |
| | | | PAVGVGDTGMEDANAVRGSASQGPLIESPADDSPPHHAPPALATPSPEEGEIQYASLSFHKARPQYPQEQEAI |
| | | | GYEYSEINIPK |
| human PECA1 | P16284 | 19 | MLLWLLLLILTPGREQSGVAPKAVLLLNPPWSTAFKGEKVALICSSISHSLAQGDTYWYHDEKLLKIKHDKIQ |
| | | | ITEPGNYQCKTRGSSLSDAVHVEFSPDWLILQALHPVFEGDNVILRCQGKDNKNTHQKVYYKDGKQLPNSYNL |
| | | | EKITVNSVSRDNSKYHCTAYRKFYILDIEVTSKPLNIQVQELFLHPVLRASSSTPIEGSPMTLTCETQLSPQR |
| | | | PDVQLQFSLFRDSQTLGLGWSRSPRLQIPAMWTEDSGSYWCEVETVTHSIKKRSLRSQIRVQRVPVSNVNLEI |
| | | | RPTGGQLIEGENMVLICSVAQGSGTVTFSWHKEGRVRSLGRKTQRSLLAELHVLTVKESDAGRYYCAADNVHS |
| | | | PILSTWIRVTVRIPVSHPVLTFRAPRAHTVVGDLLELHCESLRGSPPILYRFYHEDVTLGNSSAPSGGGASFN |
| | | | LSLTAEHSGNYSCDADNGLGAQHSHGVSLRVTVPVSRPVLTLRAPGAQAVVGDLLELHCESLRGSFPILYWFY |
| | | | HEDDTLGNISAHSGGGASFNLSLTTEHSGNYSCEADNGLGAQHSKVVTLNVTGTSRNRTGLTAAGITGLVLSI |
| | | | LVLAAAAALLHYARARRKPGGLSATGTSSHSPSECQEPSSSRPSRIDPQEPTHSKPLAPMELEPMYSNVNPGD |
| | | | SNPIYSQIWSIQHTKENSANCPMMHQEHEELTVLYSELKKTHPDDSAGEASSRGRAHEEDDEENYENVPRVLL |
| | | | ASDH |
| human SIGL9 | Q9Y336 | 20 | MAASAGAGAVIAAPDSRRWLWSVLAAALGLLTAGVSALEVYTPKEIFVANGTQGKLTCKFKSTSTTGGLTSVS |
| | | | WSFQPEGADTTVSFFHYSQGQVYLGNYPPFKDRISWAGDLDKKDASINIENMQFIHNGTYICDVKNPPDIVVQ |
| | | | PGHIRLYVVEKENLPVFPVWVVVGIVTAVVLGLTLLISMILAVLYRRKNSKRDYTGCSTSESLSPVKQAPRKS |
| | | | PSDTEGLVKSLPSGSHQGPVIYAQLDHSGGHHSDKINKSESVVYADIRKN |
| human SIGL6 | O43699 | 21 | MGRPLLLPLLPLLLPPAFLQPSGSTGSGPSYLYGVTQPKHLSASMGGSVEIPFSFYYPWELATAPDVRISWRR |
| | | | GHFHRQSFYSTRPPSIHKDYVNRLFLNWTEGQKSGFLRISNLQKQDQSVYFCRVELDTRSSGRQQWQSIEGTK |
| | | | LSITQAVTTTTQRPSSMTTTWRLSSTTTTTGLRVTQGKRRSDSWHISLETAVGVAVAVTVLGIMILGLICLLR |
| | | | WRRRKGQQRTKATTPAREPFQNTEEPYENIRNEGQNTDPKLNPKDDGIVYASLALSSSTSPRAPPSHRPLKSP |
| | | | QNETLYSVLKA |
| human CD33 | P20138 | 22 | MARAMAAAWPLLLVALLVLSWPPPGTGDVVVQAPTQVPGFLGDSVTLPCYLQVPNMEVTHVSQLTWARHGESG |
| | | | SMAVFHQTQGPSYSESKRLEFVAARLGAELRNASLRMFGLRVEDEGNYTCLFVTFPQGSRSVDIWLRVLAKPQ |
| | | | NTAEVQKVQLTGEPVPMARCVSTGGRPPAQITWHSDLGGMPNTSQVPGFLSGTVTVTSLWILVPSSQVDGKNV |
| | | | TCKVEHESFEKPQLLTVNLTVYYPPEVSISGYDNNWYLGQNEATLTCDARSNPEPTGYNWSTTMGPLPPFAVA |
| | | | QGAQLLIRPVDKPINTTLICNVTNALGARQAELTVQVKEGPPSEHSGISRNAIIFLVLGILVFLILLGIGIYF |
| | | | YWSKCSREVLWHCHLCPSSTEHASASANGHVSYSAVSRENSSSQDPQTEGTR |
| human FCRL5 | Q96RD9 | 23 | MVPGQAQPQSPEMLLLPLLLPVLGAGSLNKDPSYSLQVQRQVPVPEGLCVIVSCNLSYPRDGWDESTAAYGYW |
| | | | FKGRTSPKTGAPVATNNQSREVEMSTRDRFQLTGDPGKGSCSLVIRDAQREDEAWYFFRVERGSRVRHSFLSN |
| | | | AFFLKVTALTKKPDVYIPETLEPGQPVTVICVFNWAFKKCPAPSFSWTGAALSPRRTRPSTSHFSVLSFTPSP |
| | | | QDHDTDLTCHVDFSRKGVSAQRTVRLRVAYAPKDLIISISHDNTSALELQGNVIYLEVQKGQFLRLLCAADSQ |
| | | | PPATLSWVLQDRVLSSSHPWGPRTLGLELRGVRAGDSGRYTCRAENRLGSQQQALDLSVQYPPENLRVMVSQA |
| | | | NRTVLENLGNGTSLPVLEGQSLRLVCVTHSSPPARLSWTRWGQTVGPSQPSDPGVLELPPIQMEHEGEFTCHA |
| | | | QHPLGSQHVSLSLSVHYPPQLLGPSCSWEAEGLHCSCSSQASPAPSLRWWLGEELEGNSSQGSFEVTPSSAGP |
| | | | WANSSLSLHGGLSSGLRLRCKAWNVHGAQSGSVFQLLPGKLEHGGGLGLGAALGAGVAALLAFCSCLVVFRVK |
| | | | ICRKEARKRAAAEQDVPSTLGPISQGHQHECSAGSSQDHPPPGAATYTPGKGEEQELHYASLSFQGLRLWEPA |
| | | | DQEAPSTTEYSEIKIHTGQPLRGPGFGLQLEREMSGMVPK |
| human FCRL2 | Q96LA5 | 24 | MLLPLLLSSLLGGSQAMDGRFWIRVQESVMVPEGLCISVPCSFSYPRQDWTGSTPAYGYWFKAVTETTKGAPV |
| | | | ATNHQSREVEMSTRGRFQLTGDPAKGNCSLVIRDAQMQDESQYFFRVERGSYVRYNFMNDGFFLKVTALTQKP |
| | | | DVYIPETLEPGQPVTVICVFNWAFEECPPPSFSWTGAALSSQGTKPTTSHFSVLSFTPRPQDHNTDLTCHVDF |
| | | | SRKGVSAQRTVRLRVAYAPRDLVISISRDNTPALEPQPQGNVPYLEAQKGQFLRLLCAADSQPPATLSWVLQN |
| | | | RVLSSSHPWGPRPLGLELPGVKAGDSGRYTCRAENRLGSQQRALDLSVQYPPENLRVMVSQANRTVLENLGNG |
| | | | TSLPVLEGQSLCLVCVTHSSPPARLSWTQRGQVLSPSQPSDPGVLELPRVQVEHEGEFTCHARHPLGSQHVSL |
| | | | SLSVHYSPKLLGPSCSWEAEGLHCSCSSQASPAPSLRWWLGEELLEGNSSQDSFEVTPSSAGPWANSSLSLHG |
| | | | GLSSGLRLRCEAWNVHGAQSGSILQLPDKKGLISTAFSNGAFLGIGITALLFLCLALIIMKILPKRRTQTETP |
| | | | RPRFSRHSTILDYINVVPTAGPLAQKRNQKATPNSPRTPLPPGAPSPESKKNQKKQYQLPSFPEPKSSTQAPE |
| | | | SQESQEELHYATLNFPGVRPRPEARMPKGTQADYAEVKFQ |
| human FCRL1 | Q96LA6 | 25 | MWSHLNRLLFWSIFSSVTCRKAVLDCEAMKTNEFPSPCLDSKTKVVMKGQNVSMFCSHKNKSLQITYSLFRRK |
| | | | THLGTQDGKGEPAIFNLSITEAHESGPYKCKAQVTSCSKYSRDFSFTIVDPVTSPVLNIMVIQTETDRHITLH |
| | | | CLSVNGSLPINYTFFENHVAISPAISKYDREPAEFNLTKKNPGEEEEYRCEAKNRLPNYATYSHPVTMPSTGG |
| | | | DSCPFCLKLLLPGLLLLLVVIILILAFWVLPKYKTRKAMRNNVPRDRGDTAMEVGIYANILEKQAKEESVPEV |
| | | | GSRPCVSTAQDEAKHSQELQYATPVFQEVAPREQEACDSYKSGYVYSELNF |
| human SIG12 | Q96PQ1 | 26 | MIPTFTALLCLGLSLGPRTHMQAGPLPKPTLWAEPGSVISWGNSVTIWCQGTLEAREYRLDKEESPAPWDRQN |
| | | | PLEPKNKARFSIPSMTEDYAGRYRCYYRSPVGWSQPSDPLELVMTGAYSKPTLSALPSPLVTSGKSVTLLCQS |
| | | | RSPMDTFLLIKERAAHPLLHLRSEHGAQQHQAEFPMSPVTSVHGGTYRCFSSHGFSHYLLSHPSDPLELIVSG |
| | | | SLEDPRPSPTRSVSTAAGPEDQPLMPTGSVPHSGLRRHWEVLIGVLVVSILLLSLLLFLLLQHWRQGKHRTLA |
| | | | QRQADFQRPPGAAEPEPKDGGLQRRSSPAADVQGENFCAAVKNTQPEDGVEMDTRQSPHDEDPQAVTYAKVKH |
| | | | SRPRREMASPPSPLSGEFLDTKDRQAEEDRQMDTEAAASEAPQDVTYAQLHSFTLRQKATEPPPSQEGASPAE |
| | | | PSVYATLAIH |
| human FCRL3 | Q96P31 | 27 | MTPALTALLCLGLSLGPRTRVQAGPFPKPTLWAEPGSVISWGSPVTIWCQGSQEAQEYRLHKEGSPEPLDRNN |
| | | | PLEPKNKARFSIPSMTEHHAGRYRCHYYSSAGWSEPSDPLEMVMTGAYSKPTLSALPSPVVASGGNMTLRCGS |
| | | | QKGYHHFVLMKEGEHQLPRTLDSQQLHSRGFQALFPVGPVTPSHRWRFTCYYYYTNTPWVWSHPSDPLEILPS |
| | | | GVSRKPSLLTLQGPVLAPGQSLTLQCGSDVGYNRFVLYKEGERDFLQRPGQQPQAGLSQANFTLGPVSPSNGG |
| | | | QYRCYGAHNLSSEWSAPSDPLNILMAGQIYDTVSLSAQPGPTVASGENVTLLCQSWWQFDTFLLTKEGAAHPP |
| | | | LRLRSMYGAHKYQAEFPMSPVTSAHAGTYRCYGSYSSNPHLLSHPSEPLELVVSGHSGGSSLPPTGPPSTPGL |
| | | | GRYLEVLIGVSVAFVLLLFLLLFLLLRRQRHSKHRTSDQRKTDFQRPAGAAETEPKDRGLLRRSSPAADVQEE |
| | | | NLYAAVKDTQSEDRVELDSQSPHDEDPQAVTYAPVKHSSPRREMASPPSSLSGEFLDTKDRQVEEDRQMDTEA |
| | | | AASEASQDVTYAQLHSLTLRRKATEPPPSQEGEPPAEPSIYATLAIH |
| human MPZL1 | O95297 | 28 | MSLMVVSMACVGFFLLEGPWPHVGGQDKPFLSAWPGTVVSEGQHVTLQCRSRLGFNEFSLSKEDGMPVPELYN |
| | | | RIFRNSFLMGPVTPAHAGTYRCCSSHPHSPTGWSAPSNPVVIMVTGVHRKPSLLAHPGPLVKSGETVILQCWS |
| | | | DVRFERFLLHREGITEDPLRLVGQLHDAGSQVNYSMGPMTPALAGTYRCFGSVTHLPYELSAPSDPLDIVVVG |
| | | | LYGKPSLSAQPGPTVQAGENVTLSCSSRSLFDIYHLSREAEAGELRLTAVLRVNGTFQANFPLGPVTHGGNYR |
| | | | CFGSFRALPHAWSDPSDPLPVSVTGNSRHLHVLIGTSVVIIPFAILLFFLLHRWCANKKNAVVMDQEPAGNRT |
| | | | VNREDSDEQDPQEVTYAQLNHCVFTQRKITRPSQRPKTPPTDTSV |
| human PILRA | Q9UKJ1 | 29 | MLLLLLLLPLLWGTKGMEGDRQYGDGYLLQVQELVTVQEGLCVHVPCSFSYPQDGWTDSDPVHGYWFRAGDRP |
| | | | YQDAPVATNNPDREVQAETQGRFQLLGDIWSNDCSLSIRDARKRDKGSYFFRLERGSMKWSYKSQLNYKTKQL |
| | | | SVFVTALTHRPDILILGTLESGHSRNLTCSVPWACKQGTPPMISWIGASVSSPGPTTARSSVLTLTPKPQDHG |
| | | | TSLTCQVTLPGTGVTTTSTVRLDVSYPPWNLTMTVFQGDATASTALGNGSSLSVLEGQSLRLVCAVNSNPPAR |
| | | | LSWTRGSLTLCPSRSSNPGLLELPRVHVRDEGEFTCRAQNAQGSQHISLSLSLQNEGTGTSRPVSQVTLAAVG |
| | | | GAGATALAFLSFCIIFIIVRSCRKKSARPAAGVGDTGMEDAKAIRGSASQGPLTESWKDGNPLKKPPPAVAPS |
| | | | SGEEGELHYATLSFHKVKPQDPQGQEATDSEYSEIKIHKRETAETQACLRNHNPSSKEVRG |
| human PVR | P15151 | 30 | MLLLLLLPLLWGRERVEGQKSNRKDYSLTMQSSVTVQEGMCVHVRCSFSYPVDSQTDSDPVHGYWFRAGNDIS |
| | | | WKAPVATNNPAWAVQEETRDRFHLLGDPQTKNCTLSIRDARMSDAGRYFFRMEKGNIKWNYKYDQLSVNVTAL |
| | | | THRPNILIPGTLESGCFQNLTCSVPWACEQGTPPMISWMGTSVSPLHPSTTRSSVLTLIPQPQHHGTSLTCQV |
| | | | TLPGAGVTTNRTIQLNVSYPPQNLTVTVFQGEGTASTALGNSSSLSVLEGQSLRLVCAVDSNPPARLSWTWRS |
| | | | LTLYPSQPSNPLVLELQVHLGDEGEFTCRAQNSLGSQHVSLNLSLQQEYTGKMRPVSGVLLGAVGGAGATALV |
| | | | FLSFCVIFIVVRSCRKKSARPAADVGDIGMKDANTIRGSASQGNLTESWADDNPRHHGLAAHSSGEEREIQYA |
| | | | PLSFHKGEPQDLSGQEATNNEYSEIKIPK |
| human SIG11 | Q96RL6 | 31 | MTLTLSVLICLGLSVGPRTCVQAGTLPKPTLWAEPASVIARGKPVTLWCQGPLETEEYRLDKEGLPWARKRQN |
| | | | PLEPGAKAKFHIPSTVYDSAGRYRCYYETPAGWSEPSDPLELVATGFYAEPTLLALPSPVVASGGNVTLQCDT |
| | | | LDGLLTFVLVEEEQKLPRTLYSQKLPKGPSQALFPVGPVTPSCRWRFRCYYYYRKNPQVWSNPSDLLEILVPG |
| | | | VSRKPSLLIPQGSVVARGGSLTLQCRSDVGYDIFVLYKEGEHDLVQGSGQQPQAGLSQANFTLGPVSRSHGGQ |
| | | | YRCYGAHNLSPRWSAPSDPLDILIAGLIPDIPALSVQPGPKVASGENVTLLCQSWHQIDTFFLTKEGAAHPPL |
| | | | CLKSKYQSYRHQAEFSMSPVTSAQGGTYRCYSAIRSYPYLLSSPSYPQELVVSGPSGDPSLSPTGSTPTPGPE |
| | | | DQPLTPTGLDPQSGLGRHLGVVTGVSVAFVLLLFLLLFLLLRHRHQSKHRTSAHFYRPAGAAGPEPKDQGLQK |
| | | | RASPVADIQEEILNAAVKDTQPKDGVEMDARAAASEAPQDVTYAQLHSLTLRREATEPPPSQEREPPAEPSIY |
| | | | APLAIH |
| human SIG10 | Q96LC7 | 32 | MTPIVTVLICLGLSLGPRTHVQTGTIPKPTLWAEPDSVITQGSPVTLSCQGSLEAQEYRLYREKKSASWITRI |
| | | | RPELVKNGQFHIPSITWEHTGRYGCQYYSRARWSELSDPLVLVMTGAYPKPTLSAQPSPVVTSGGRVTLQCES |
| | | | QVAFGGFILCKEGEEEHPQCLNSQPHARGSSRAIFSVGPVSPNRRWSHRCYGYDLNSPYVWSSPSDLLELLVP |
| | | | GVSKKPSLSVQPGPVVAPGESLTLQCVSDVGYDRFVLYKEGERDLRQLPGRQPQAGLSQANFTLGPVSRSYGG |
| | | | QYRCYGAHNLSSECSAPSDPLDILITGQIRGTPFISVQPGPTVASGENVTLLCQSWRQFHTFLLTKAGAADAP |
| | | | LRLRSIHEYPKYQAEFPMSPVTSAHAGTYRCYGSLNSDPYLLSHPSEPLELVVSGPSMGSSPPPTGPISTPAG |
| | | | PEDQPLTPTGSDPQSGLGRHLGVVIGILVAVVLLLLLLLLLFLILRHRRQGKHWTSTQRKADFQHPAGAVGPE |
| | | | PTDRGLQWRSSPAADAQEENLYAAVKDTQPEDGVEMDTRAAASEAPQDVTYAQLHSLTLRRKATEPPPSQERE |
| | | | PPAEPSIYATLAIH |

TABLE 2
Example of N-CARs according to the invention

| Plasmid encoding | | | | | | | Amino acids of the |
|---|---|---|---|---|---|---|---|
| N-CAR | signal peptide | GS linker 1 | VH chain | GS linker | VL chain | GS linker 2 | inhibitory polypeptide used |
| pCLS27446 | SEQ ID | SEQ ID | SEQ ID | SEQ ID | SEQ ID | SEQ ID | 229-325 of SEQ ID NO. 36 |
| (SEQ ID NO. 100) | NO. 37 | NO. 39 | NO. 41 | NO. 38 | NO. 42 | NO. 40 | |
| pCLS27447 | SEQ ID | SEQ ID | SEQ ID | SEQ ID | SEQ ID | SEQ ID | 230-468 of SEQ ID NO. 34 |
| (SEQ ID NO. 101) | NO. 37 | NO. 39 | NO. 41 | NO. 38 | NO. 42 | NO. 40 | |
| pCLS27448 | SEQ ID | SEQ ID | SEQ ID | SEQ ID | SEQ ID | SEQ ID | 179-440 of SEQ ID NO. 35 |
| (SEQ ID NO. 102) | NO. 37 | NO. 39 | NO. 41 | NO. 38 | NO. 42 | NO. 40 | |
| pCLS27449 | SEQ ID | SEQ ID | SEQ ID | SEQ ID | SEQ ID | SEQ ID | 181-386 of SEQ ID NO. 33 |
| (SEQ ID NO. 103) | NO. 37 | NO. 39 | NO. 41 | NO. 38 | NO. 42 | NO. 40 | |
| pCLS27450 | SEQ ID | SEQ ID | SEQ ID | SEQ ID | SEQ ID | SEQ ID | 229-364 of SEQ ID NO. 22 |
| (SEQ ID NO. 104) | NO. 37 | NO. 39 | NO. 41 | NO. 38 | NO. 42 | NO. 40 | |
| pCLS27451 | SEQ ID | SEQ ID | SEQ ID | SEQ ID | SEQ ID | SEQ ID | 214-310 of SEQ ID NO. 3 |
| (SEQ ID NO. 105) | NO. 37 | NO. 39 | NO. 41 | NO. 38 | NO. 42 | NO. 40 | |
| pCLS27452 | SEQ ID | SEQ ID | SEQ ID | SEQ ID | SEQ ID | SEQ ID | 292-429 of SEQ ID NO. 25 |
| (SEQ ID NO. 106) | NO. 37 | NO. 39 | NO. 41 | NO. 38 | NO. 42 | NO. 40 | |
| pCLS27453 | SEQ ID | SEQ ID | SEQ ID | SEQ ID | SEQ ID | SEQ ID | 388-508 of SEQ ID NO. 24 |
| (SEQ ID NO. 107) | NO. 37 | NO. 39 | NO. 41 | NO. 38 | NO. 42 | NO. 40 | |
| pCLS27454 | SEQ ID | SEQ ID | SEQ ID | SEQ ID | SEQ ID | SEQ ID | 564-734 of SEQ ID NO. 27 |
| (SEQ ID NO. 108) | NO. 37 | NO. 39 | NO. 41 | NO. 38 | NO. 42 | NO. 40 | |
| pCLS27455 | SEQ ID | SEQ ID | SEQ ID | SEQ ID | SEQ ID | SEQ ID | 375-515 of SEQ ID NO. 18 |
| (SEQ ID NO. 109) | NO. 37 | NO. 39 | NO. 41 | NO. 38 | NO. 42 | NO. 40 | |
| pCLS27456 | SEQ ID | SEQ ID | SEQ ID | SEQ ID | SEQ ID | SEQ ID | 753-977 of SEQ ID NO. 23 |
| (SEQ ID NO. 110) | NO. 37 | NO. 39 | NO. 41 | NO. 38 | NO. 42 | NO. 40 | |
| pCLS27457 | SEQ ID | SEQ ID | SEQ ID | SEQ ID | SEQ ID | SEQ ID | 420-598 of SEQ ID NO. 16 |
| (SEQ ID NO. 111) | NO. 37 | NO. 39 | NO. 41 | NO. 38 | NO. 42 | NO. 40 | |
| pCLS27445 | SEQ ID | SEQ ID | SEQ ID | SEQ ID | SEQ ID | SEQ ID | 420-631 of SEQ ID NO. 11 |
| (SEQ ID NO. 112) | NO. 37 | NO. 39 | NO. 41 | NO. 38 | NO. 42 | NO. 40 | |
| pCLS27458 | SEQ ID | SEQ ID | SEQ ID | SEQ ID | SEQ ID | SEQ ID | 219-448 of SEQ ID NO. 10 |
| (SEQ ID NO. 113) | NO. 37 | NO. 39 | NO. 41 | NO. 38 | NO. 42 | NO. 40 | |
| pCLS27459 | SEQ ID | SEQ ID | SEQ ID | SEQ ID | SEQ ID | SEQ ID | 419-590 of SEQ ID NO. 15 |
| (SEQ ID NO. 114) | NO. 37 | NO. 39 | NO. 41 | NO. 38 | NO. 42 | NO. 40 | |
| pCLS27460 | SEQ ID | SEQ ID | SEQ ID | SEQ ID | SEQ ID | SEQ ID | 214-343 of SEQ ID NO. 9 |
| (SEQ ID NO. 115) | NO. 37 | NO. 39 | NO. 41 | NO. 38 | NO. 42 | NO. 40 | |
| pCLS27461 | SEQ ID | SEQ ID | SEQ ID | SEQ ID | SEQ ID | SEQ ID | 147-269 of SEQ ID NO. 28 |
| (SEQ ID NO. 116) | NO. 37 | NO. 39 | NO. 41 | NO. 38 | NO. 42 | NO. 40 | |
| pCLS27462 | SEQ ID | SEQ ID | SEQ ID | SEQ ID | SEQ ID | SEQ ID | 151-303 of SEQ ID NO. 29 |
| (SEQ ID NO. 117) | NO. 37 | NO. 39 | NO. 41 | NO. 38 | NO. 42 | NO. 40 | |
| pCLS27464 | SEQ ID | SEQ ID | SEQ ID | SEQ ID | SEQ ID | SEQ ID | 329-417 of SEQ ID NO. 30 |
| (SEQ ID NO. 118) | NO. 37 | NO. 39 | NO. 41 | NO. 38 | NO. 42 | NO. 40 | |
| pCLS27465 | SEQ ID | SEQ ID | SEQ ID | SEQ ID | SEQ ID | SEQ ID | 442-697 of SEQ ID NO. 32 |
| (SEQ ID NO. 119) | NO. 37 | NO. 39 | NO. 41 | NO. 38 | NO. 42 | NO. 40 | |
| pCLS27466 | SEQ ID | SEQ ID | SEQ ID | SEQ ID | SEQ ID | SEQ ID | 453-698 of SEQ ID NO. 31 |
| (SEQ ID NO. 120) | NO. 37 | NO. 39 | NO. 41 | NO. 38 | NO. 42 | NO. 40 | |
| pCLS27467 | SEQ ID | SEQ ID | SEQ ID | SEQ ID | SEQ ID | SEQ ID | 463-595 of SEQ ID NO. 26 |
| (SEQ ID NO. 121) | NO. 37 | NO. 39 | NO. 41 | NO. 38 | NO. 42 | NO. 40 | |
| pCLS27468 | SEQ ID | SEQ ID | SEQ ID | SEQ ID | SEQ ID | SEQ ID | 331-551 of SEQ ID NO. 17 |
| (SEQ ID NO. 122) | NO. 37 | NO. 39 | NO. 41 | NO. 38 | NO. 42 | NO. 40 | |
| pCLS27469 | SEQ ID | SEQ ID | SEQ ID | SEQ ID | SEQ ID | SEQ ID | 334-453 of SEQ ID NO. 21 |
| (SEQ ID NO. 123) | NO. 37 | NO. 39 | NO. 41 | NO. 38 | NO. 42 | NO. 40 | |
| pCLS27470 | SEQ ID | SEQ ID | SEQ ID | SEQ ID | SEQ ID | SEQ ID | 337-467 of SEQ ID NO. 14 |
| (SEQ ID NO. 124) | NO. 37 | NO. 39 | NO. 41 | NO. 38 | NO. 42 | NO. 40 | |
| pCLS27471 | SEQ ID | SEQ ID | SEQ ID | SEQ ID | SEQ ID | SEQ ID | 345-499 of SEQ ID NO. 13 |
| (SEQ ID NO. 125) | NO. 37 | NO. 39 | NO. 41 | NO. 38 | NO. 42 | NO. 40 | |
| pCLS27472 | SEQ ID | SEQ ID | SEQ ID | SEQ ID | SEQ ID | SEQ ID | 237-463 of SEQ ID NO. 20 |
| (SEQ ID NO. 126) | NO. 37 | NO. 39 | NO. 41 | NO. 38 | NO. 42 | NO. 40 | |
| pCLS27473 | SEQ ID | SEQ ID | SEQ ID | SEQ ID | SEQ ID | SEQ ID | 206-348 of SEQ ID NO. 2 |
| (SEQ ID NO. 127) | NO. 37 | NO. 39 | NO. 41 | NO. 38 | NO. 42 | NO. 40 | |
| pCLS27474 | SEQ ID | SEQ ID | SEQ ID | SEQ ID | SEQ ID | SEQ ID | 206-348 of SEQ ID NO. 1 |
| (SEQ ID NO. 128) | NO. 37 | NO. 39 | NO. 41 | NO. 38 | NO. 42 | NO. 40 | |
| pCLS27475 | SEQ ID | SEQ ID | SEQ ID | SEQ ID | SEQ ID | SEQ ID | 206-341 of SEQ ID NO. 4 |
| (SEQ ID NO. 129) | NO. 37 | NO. 39 | NO. 41 | NO. 38 | NO. 42 | NO. 40 | |
| pCLS27476 | SEQ ID | SEQ ID | SEQ ID | SEQ ID | SEQ ID | SEQ ID | 203-377 of SEQ ID NO. 6 |
| (SEQ ID NO. 130) | NO. 37 | NO. 39 | NO. 41 | NO. 38 | NO. 42 | NO. 40 | |
| pCLS27477 | SEQ ID | SEQ ID | SEQ ID | SEQ ID | SEQ ID | SEQ ID | 201-375 of SEQ ID NO. 8 |
| (SEQ ID NO. 131) | NO. 37 | NO. 39 | NO. 41 | NO. 38 | NO. 42 | NO. 40 | |
| pCLS27478 | SEQ ID | SEQ ID | SEQ ID | SEQ ID | SEQ ID | SEQ ID | 301-444 of SEQ ID NO. 7 |
| (SEQ ID NO. 132) | NO. 37 | NO. 39 | NO. 41 | NO. 38 | NO. 42 | NO. 40 | |
| pCLS27479 | SEQ ID | SEQ ID | SEQ ID | SEQ ID | SEQ ID | SEQ ID | 301-455 of SEQ ID NO. 5 |
| (SEQ ID NO. 133) | NO. 37 | NO. 39 | NO. 41 | NO. 38 | NO. 42 | NO. 40 | |
| pCLS27480 | SEQ ID | SEQ ID | SEQ ID | SEQ ID | SEQ ID | SEQ ID | 296-410 of SEQ ID NO. 12 |
| (SEQ ID NO. 134) | NO. 37 | NO. 39 | NO. 41 | NO. 38 | NO. 42 | NO. 40 | |
| pCLS27481 | SEQ ID | SEQ ID | SEQ ID | SEQ ID | SEQ ID | SEQ ID | 592-738 of SEQ ID NO. 19 |
| (SEQ ID NO. 135) | NO. 37 | NO. 39 | NO. 41 | NO. 38 | NO. 42 | NO. 40 | |
| pCLS27482 | SEQ ID | SEQ ID | SEQ ID | SEQ ID | SEQ ID | SEQ ID | 229-325 of SEQ ID NO. 36 |
| (SEQ ID NO. 138) | NO. 37 | NO. 39 | NO. 43 | NO. 38 | NO. 44 | NO. 40 | |
| pCLS27483 | SEQ ID | SEQ ID | SEQ ID | SEQ ID | SEQ ID | SEQ ID | 230-468 of SEQ ID NO. 34 |
| (SEQ ID NO. 139) | NO. 37 | NO. 39 | NO. 43 | NO. 38 | NO. 44 | NO. 40 | |
| pCLS27484 | SEQ ID | SEQ ID | SEQ ID | SEQ ID | SEQ ID | SEQ ID | 179-440 of SEQ ID NO. 35 |
| (SEQ ID NO. 140) | NO. 37 | NO. 39 | NO. 43 | NO. 38 | NO. 44 | NO. 40 | |
| pCLS27485 | SEQ ID | SEQ ID | SEQ ID | SEQ ID | SEQ ID | SEQ ID | 181-386 of SEQ ID NO. 33 |
| (SEQ ID NO. 141) | NO. 37 | NO. 39 | NO. 43 | NO. 38 | NO. 44 | NO. 40 | |
| pCLS27486 | SEQ ID | SEQ ID | SEQ ID | SEQ ID | SEQ ID | SEQ ID | 229-364 of SEQ ID NO. 22 |
| (SEQ ID NO. 142) | NO. 37 | NO. 39 | NO. 43 | NO. 38 | NO. 44 | NO. 40 | |
| pCLS27487 | SEQ ID | SEQ ID | SEQ ID | SEQ ID | SEQ ID | SEQ ID | 214-310 of SEQ ID NO. 3 |
| (SEQ ID NO. 143) | NO. 37 | NO. 39 | NO. 43 | NO. 38 | NO. 44 | NO. 40 | |
| pCLS27488 | SEQ ID | SEQ ID | SEQ ID | SEQ ID | SEQ ID | SEQ ID | 292-429 of SEQ ID NO. 25 |
| (SEQ ID NO. 144) | NO. 37 | NO. 39 | NO. 43 | NO. 38 | NO. 44 | NO. 40 | |
| pCLS27489 | SEQ ID | SEQ ID | SEQ ID | SEQ ID | SEQ ID | SEQ ID | 388-508 of SEQ ID NO. 24 |
| (SEQ ID NO. 145) | NO. 37 | NO. 39 | NO. 43 | NO. 38 | NO. 44 | NO. 40 | |
| pCLS27490 | SEQ ID | SEQ ID | SEQ ID | SEQ ID | SEQ ID | SEQ ID | 564-734 of SEQ ID NO. 27 |
| (SEQ ID NO. 146) | NO. 37 | NO. 39 | NO. 43 | NO. 38 | NO. 44 | NO. 40 | |
| pCLS27491 | SEQ ID | SEQ ID | SEQ ID | SEQ ID | SEQ ID | SEQ ID | 375-515 of SEQ ID NO. 18 |
| (SEQ ID NO. 147) | NO. 37 | NO. 39 | NO. 43 | NO. 38 | NO. 44 | NO. 40 | |
| pCLS27492 | SEQ ID | SEQ ID | SEQ ID | SEQ ID | SEQ ID | SEQ ID | 753-977 of SEQ ID NO. 23 |
| (SEQ ID NO. 148) | NO. 37 | NO. 39 | NO. 43 | NO. 38 | NO. 44 | NO. 40 | |
| pCLS27493 | SEQ ID | SEQ ID | SEQ ID | SEQ ID | SEQ ID | SEQ ID | 420-598 of SEQ ID NO. 16 |
| (SEQ ID NO. 149) | NO. 37 | NO. 39 | NO. 43 | NO. 38 | NO. 44 | NO. 40 | |
| pCLS27494 | SEQ ID | SEQ ID | SEQ ID | SEQ ID | SEQ ID | SEQ ID | 420-631 of SEQ ID NO. 11 |
| (SEQ ID NO. 150) | NO. 37 | NO. 39 | NO. 43 | NO. 38 | NO. 44 | NO. 40 | |
| pCLS27495 | SEQ ID | SEQ ID | SEQ ID | SEQ ID | SEQ ID | SEQ ID | 219-448 of SEQ ID NO. 10 |
| (SEQ ID NO. 151) | NO. 37 | NO. 39 | NO. 43 | NO. 38 | NO. 44 | NO. 40 | |
| pCLS27496 | SEQ ID | SEQ ID | SEQ ID | SEQ ID | SEQ ID | SEQ ID | 419-590 of SEQ ID NO. 15 |
| (SEQ ID NO. 152) | NO. 37 | NO. 39 | NO. 43 | NO. 38 | NO. 44 | NO. 40 | |
| pCLS27497 | SEQ ID | SEQ ID | SEQ ID | SEQ ID | SEQ ID | SEQ ID | 214-343 of SEQ ID NO. 9 |
| (SEQ ID NO. 153) | NO. 37 | NO. 39 | NO. 43 | NO. 38 | NO. 44 | NO. 40 | |
| pCLS27498 | SEQ ID | SEQ ID | SEQ ID | SEQ ID | SEQ ID | SEQ ID | 147-269 of SEQ ID NO. 28 |
| (SEQ ID NO. 154) | NO. 37 | NO. 39 | NO. 43 | NO. 38 | NO. 44 | NO. 40 | |
| pCLS27499 | SEQ ID | SEQ ID | SEQ ID | SEQ ID | SEQ ID | SEQ ID | 151-303 of SEQ ID NO. 29 |
| (SEQ ID NO. 155) | NO. 37 | NO. 39 | NO. 43 | NO. 38 | NO. 44 | NO. 40 | |
| pCLS27501 | SEQ ID | SEQ ID | SEQ ID | SEQ ID | SEQ ID | SEQ ID | 329-417 of SEQ ID NO. 30 |
| (SEQ ID NO. 156) | NO. 37 | NO. 39 | NO. 43 | NO. 38 | NO. 44 | NO. 40 | |
| pCLS27502 | SEQ ID | SEQ ID | SEQ ID | SEQ ID | SEQ ID | SEQ ID | 442-697 of SEQ ID NO. 32 |
| (SEQ ID NO. 157) | NO. 37 | NO. 39 | NO. 43 | NO. 38 | NO. 44 | NO. 40 | |
| pCLS27503 | SEQ ID | SEQ ID | SEQ ID | SEQ ID | SEQ ID | SEQ ID | 453-698 of SEQ ID NO. 31 |
| (SEQ ID NO. 158) | NO. 37 | NO. 39 | NO. 43 | NO. 38 | NO. 44 | NO. 40 | |
| pCLS27504 | SEQ ID | SEQ ID | SEQ ID | SEQ ID | SEQ ID | SEQ ID | 463-595 of SEQ ID NO. 26 |
| (SEQ ID NO. 159) | NO. 37 | NO. 39 | NO. 43 | NO. 38 | NO. 44 | NO. 40 | |
| pCLS27505 | SEQ ID | SEQ ID | SEQ ID | SEQ ID | SEQ ID | SEQ ID | 331-551 of SEQ ID NO. 17 |
| (SEQ ID NO. 160) | NO. 37 | NO. 39 | NO. 43 | NO. 38 | NO. 44 | NO. 40 | |
| pCLS27506 | SEQ ID | SEQ ID | SEQ ID | SEQ ID | SEQ ID | SEQ ID | 334-453 of SEQ ID NO. 21 |
| (SEQ ID NO. 161) | NO. 37 | NO. 39 | NO. 43 | NO. 38 | NO. 44 | NO. 40 | |
| pCLS27507 | SEQ ID | SEQ ID | SEQ ID | SEQ ID | SEQ ID | SEQ ID | 337-467 of SEQ ID NO. 14 |
| (SEQ ID NO. 162) | NO. 37 | NO. 39 | NO. 43 | NO. 38 | NO. 44 | NO. 40 | |
| pCLS27508 | SEQ ID | SEQ ID | SEQ ID | SEQ ID | SEQ ID | SEQ ID | 345-499 of SEQ ID NO. 13 |
| (SEQ ID NO. 163) | NO. 37 | NO. 39 | NO. 43 | NO. 38 | NO. 44 | NO. 40 | |
| pCLS27509 | SEQ ID | SEQ ID | SEQ ID | SEQ ID | SEQ ID | SEQ ID | 237-463 of SEQ ID NO. 20 |
| (SEQ ID NO. 164) | NO. 37 | NO. 39 | NO. 43 | NO. 38 | NO. 44 | NO. 40 | |
| pCLS27510 | SEQ ID | SEQ ID | SEQ ID | SEQ ID | SEQ ID | SEQ ID | 206-348 of SEQ ID NO. 2 |
| (SEQ ID NO. 165) | NO. 37 | NO. 39 | NO. 43 | NO. 38 | NO. 44 | NO. 40 | |
| pCLS27511 | SEQ ID | SEQ ID | SEQ ID | SEQ ID | SEQ ID | SEQ ID | 206-348 of SEQ ID NO. 1 |
| (SEQ ID NO. 166) | NO. 37 | NO. 39 | NO. 43 | NO. 38 | NO. 44 | NO. 40 | |
| pCLS27512 | SEQ ID | SEQ ID | SEQ ID | SEQ ID | SEQ ID | SEQ ID | 206-341 of SEQ ID NO. 4 |
| (SEQ ID NO. 167) | NO. 37 | NO. 39 | NO. 43 | NO. 38 | NO. 44 | NO. 40 | |
| pCLS27513 | SEQ ID | SEQ ID | SEQ ID | SEQ ID | SEQ ID | SEQ ID | 203-377 of SEQ ID NO. 6 |
| (SEQ ID NO. 168) | NO. 37 | NO. 39 | NO. 43 | NO. 38 | NO. 44 | NO. 40 | |
| pCLS27514 | SEQ ID | SEQ ID | SEQ ID | SEQ ID | SEQ ID | SEQ ID | 201-375 of SEQ ID NO. 8 |
| (SEQ ID NO. 169) | NO. 37 | NO. 39 | NO. 43 | NO. 38 | NO. 44 | NO. 40 | |
| pCLS27515 | SEQ ID | SEQ ID | SEQ ID | SEQ ID | SEQ ID | SEQ ID | 301-444 of SEQ ID NO. 7 |
| (SEQ ID NO. 170) | NO. 37 | NO. 39 | NO. 43 | NO. 38 | NO. 44 | NO. 40 | |
| pCLS27516 | SEQ ID | SEQ ID | SEQ ID | SEQ ID | SEQ ID | SEQ ID | 301-455 of SEQ ID NO. 5 |
| (SEQ ID NO. 171) | NO. 37 | NO. 39 | NO. 43 | NO. 38 | NO. 44 | NO. 40 | |
| pCLS27517 | SEQ ID | SEQ ID | SEQ ID | SEQ ID | SEQ ID | SEQ ID | 296-410 of SEQ ID NO. 12 |
| (SEQ ID NO. 172) | NO. 37 | NO. 39 | NO. 43 | NO. 38 | NO. 44 | NO. 40 | |
| pCLS27518 | SEQ ID | SEQ ID | SEQ ID | SEQ ID | SEQ ID | SEQ ID | 592-738 of SEQ ID NO. 19 |
| (SEQ ID NO. 173) | NO. 37 | NO. 39 | NO. 43 | NO. 38 | NO. 44 | NO. 40 | |
| pCLS27698 | SEQ ID | SEQ ID | SEQ ID | SEQ ID | SEQ ID | SEQ ID | 229-325 of SEQ ID NO. 36 |
| (SEQ ID NO. 175) | NO. 37 | NO. 39 | NO. 45 | NO. 38 | NO. 46 | NO. 40 | |
| pCLS27699 | SEQ ID | SEQ ID | SEQ ID | SEQ ID | SEQ ID | SEQ ID | 230-468 of SEQ ID NO. 34 |
| (SEQ ID NO. 176) | NO. 37 | NO. 39 | NO. 45 | NO. 38 | NO. 46 | NO. 40 | |
| pCLS27700 | SEQ ID | SEQ ID | SEQ ID | SEQ ID | SEQ ID | SEQ ID | 179-440 of SEQ ID NO. 35 |
| (SEQ ID NO. 177) | NO. 37 | NO. 39 | NO. 45 | NO. 38 | NO. 46 | NO. 40 | |
| pCLS27701 | SEQ ID | SEQ ID | SEQ ID | SEQ ID | SEQ ID | SEQ ID | 181-386 of SEQ ID NO. 33 |
| (SEQ ID NO. 178) | NO. 37 | NO. 39 | NO. 45 | NO. 38 | NO. 46 | NO. 40 | |
| pCLS27702 | SEQ ID | SEQ ID | SEQ ID | SEQ ID | SEQ ID | SEQ ID | 229-364 of SEQ ID NO. 22 |
| (SEQ ID NO. 179) | NO. 37 | NO. 39 | NO. 45 | NO. 38 | NO. 46 | NO. 40 | |
| pCLS27703 | SEQ ID | SEQ ID | SEQ ID | SEQ ID | SEQ ID | SEQ ID | 214-310 of SEQ ID NO. 3 |
| (SEQ ID NO. 180) | NO. 37 | NO. 39 | NO. 45 | NO. 38 | NO. 46 | NO. 40 | |
| pCLS27704 | SEQ ID | SEQ ID | SEQ ID | SEQ ID | SEQ ID | SEQ ID | 292-429 of SEQ ID NO. 25 |
| (SEQ ID NO. 181) | NO. 37 | NO. 39 | NO. 45 | NO. 38 | NO. 46 | NO. 40 | |
| pCLS27705 | SEQ ID | SEQ ID | SEQ ID | SEQ ID | SEQ ID | SEQ ID | 388-508 of SEQ ID NO. 24 |
| (SEQ ID NO. 182) | NO. 37 | NO. 39 | NO. 45 | NO. 38 | NO. 46 | NO. 40 | |
| pCLS27706 | SEQ ID | SEQ ID | SEQ ID | SEQ ID | SEQ ID | SEQ ID | 564-734 of SEQ ID NO. 27 |
| (SEQ ID NO. 183) | NO. 37 | NO. 39 | NO. 45 | NO. 38 | NO. 46 | NO. 40 | |
| pCLS27707 | SEQ ID | SEQ ID | SEQ ID | SEQ ID | SEQ ID | SEQ ID | 375-515 of SEQ ID NO. 18 |
| (SEQ ID NO. 184) | NO. 37 | NO. 39 | NO. 45 | NO. 38 | NO. 46 | NO. 40 | |
| pCLS27708 | SEQ ID | SEQ ID | SEQ ID | SEQ ID | SEQ ID | SEQ ID | 753-977 of SEQ ID NO. 23 |
| (SEQ ID NO. 185) | NO. 37 | NO. 39 | NO. 45 | NO. 38 | NO. 46 | NO. 40 | |
| pCLS27709 | SEQ ID | SEQ ID | SEQ ID | SEQ ID | SEQ ID | SEQ ID | 420-598 of SEQ ID NO. 16 |
| (SEQ ID NO. 186) | NO. 37 | NO. 39 | NO. 45 | NO. 38 | NO. 46 | NO. 40 | |
| pCLS27710 | SEQ ID | SEQ ID | SEQ ID | SEQ ID | SEQ ID | SEQ ID | 420-631 of SEQ ID NO. 11 |
| (SEQ ID NO. 187) | NO. 37 | NO. 39 | NO. 45 | NO. 38 | NO. 46 | NO. 40 | |
| pCLS27711 | SEQ ID | SEQ ID | SEQ ID | SEQ ID | SEQ ID | SEQ ID | 219-448 of SEQ ID NO. 10 |
| (SEQ ID NO. 188) | NO. 37 | NO. 39 | NO. 45 | NO. 38 | NO. 46 | NO. 40 | |
| pCLS27712 | SEQ ID | SEQ ID | SEQ ID | SEQ ID | SEQ ID | SEQ ID | 419-590 of SEQ ID NO. 15 |
| (SEQ ID NO. 189) | NO. 37 | NO. 39 | NO. 45 | NO. 38 | NO. 46 | NO. 40 | |
| pCLS27713 | SEQ ID | SEQ ID | SEQ ID | SEQ ID | SEQ ID | SEQ ID | 214-343 of SEQ ID NO. 9 |
| (SEQ ID NO. 190) | NO. 37 | NO. 39 | NO. 45 | NO. 38 | NO. 46 | NO. 40 | |
| pCLS27714 | SEQ ID | SEQ ID | SEQ ID | SEQ ID | SEQ ID | SEQ ID | 147-269 of SEQ ID NO. 28 |
| (SEQ ID NO. 191) | NO. 37 | NO. 39 | NO. 45 | NO. 38 | NO. 46 | NO. 40 | |
| pCLS27715 | SEQ ID | SEQ ID | SEQ ID | SEQ ID | SEQ ID | SEQ ID | 151-303 of SEQ ID NO. 29 |
| (SEQ ID NO. 192) | NO. 37 | NO. 39 | NO. 45 | NO. 38 | NO. 46 | NO. 40 | |
| pCLS27716 | SEQ ID | SEQ ID | SEQ ID | SEQ ID | SEQ ID | SEQ ID | 329-417 of SEQ ID NO. 30 |
| (SEQ ID NO. 193) | NO. 37 | NO. 39 | NO. 45 | NO. 38 | NO. 46 | NO. 40 | |
| pCLS27717 | SEQ ID | SEQ ID | SEQ ID | SEQ ID | SEQ ID | SEQ ID | 442-697 of SEQ ID NO. 32 |
| (SEQ ID NO. 194) | NO. 37 | NO. 39 | NO. 45 | NO. 38 | NO. 46 | NO. 40 | |
| pCLS27718 | SEQ ID | SEQ ID | SEQ ID | SEQ ID | SEQ ID | SEQ ID | 453-698 of SEQ ID NO. 31 |
| (SEQ ID NO. 195) | NO. 37 | NO. 39 | NO. 45 | NO. 38 | NO. 46 | NO. 40 | |
| pCLS27719 | SEQ ID | SEQ ID | SEQ ID | SEQ ID | SEQ ID | SEQ ID | 463-595 of SEQ ID NO. 26 |
| (SEQ ID NO. 196) | NO. 37 | NO. 39 | NO. 45 | NO. 38 | NO. 46 | NO. 40 | |
| pCLS27720 | SEQ ID | SEQ ID | SEQ ID | SEQ ID | SEQ ID | SEQ ID | 331-551 of SEQ ID NO. 17 |
| (SEQ ID NO. 197) | NO. 37 | NO. 39 | NO. 45 | NO. 38 | NO. 46 | NO. 40 | |
| pCLS27721 | SEQ ID | SEQ ID | SEQ ID | SEQ ID | SEQ ID | SEQ ID | 334-453 of SEQ ID NO. 21 |
| (SEQ ID NO. 198) | NO. 37 | NO. 39 | NO. 45 | NO. 38 | NO. 46 | NO. 40 | |
| pCLS27722 | SEQ ID | SEQ ID | SEQ ID | SEQ ID | SEQ ID | SEQ ID | 337-467 of SEQ ID NO. 14 |
| (SEQ ID NO. 199) | NO. 37 | NO. 39 | NO. 45 | NO. 38 | NO. 46 | NO. 40 | |
| pCLS27723 | SEQ ID | SEQ ID | SEQ ID | SEQ ID | SEQ ID | SEQ ID | 345-499 of SEQ ID NO. 13 |
| (SEQ ID NO. 200) | NO. 37 | NO. 39 | NO. 45 | NO. 38 | NO. 46 | NO. 40 | |
| pCLS27724 | SEQ ID | SEQ ID | SEQ ID | SEQ ID | SEQ ID | SEQ ID | 237-463 of SEQ ID NO. 20 |
| (SEQ ID NO. 201) | NO. 37 | NO. 39 | NO. 45 | NO. 38 | NO. 46 | NO. 40 | |
| pCLS27725 | SEQ ID | SEQ ID | SEQ ID | SEQ ID | SEQ ID | SEQ ID | 206-348 of SEQ ID NO. 2 |
| (SEQ ID NO. 202) | NO. 37 | NO. 39 | NO. 45 | NO. 38 | NO. 46 | NO. 40 | |
| pCLS27726 | SEQ ID | SEQ ID | SEQ ID | SEQ ID | SEQ ID | SEQ ID | 206-348 of SEQ ID NO. 1 |
| (SEQ ID NO. 203) | NO. 37 | NO. 39 | NO. 45 | NO. 38 | NO. 46 | NO. 40 | |
| pCLS27727 | SEQ ID | SEQ ID | SEQ ID | SEQ ID | SEQ ID | SEQ ID | 206-341 of SEQ ID NO. 4 |
| (SEQ ID NO. 204) | NO. 37 | NO. 39 | NO. 45 | NO. 38 | NO. 46 | NO. 40 | |
| pCLS27728 | SEQ ID | SEQ ID | SEQ ID | SEQ ID | SEQ ID | SEQ ID | 203-377 of SEQ ID NO. 6 |
| (SEQ ID NO. 205) | NO. 37 | NO. 39 | NO. 45 | NO. 38 | NO. 46 | NO. 40 | |
| pCLS27729 | SEQ ID | SEQ ID | SEQ ID | SEQ ID | SEQ ID | SEQ ID | 201-375 of SEQ ID NO. 8 |
| (SEQ ID NO. 206) | NO. 37 | NO. 39 | NO. 45 | NO. 38 | NO. 46 | NO. 40 | |
| pCLS27730 | SEQ ID | SEQ ID | SEQ ID | SEQ ID | SEQ ID | SEQ ID | 301-444 of SEQ ID NO. 7 |
| (SEQ ID NO. 207) | NO. 37 | NO. 39 | NO. 45 | NO. 38 | NO. 46 | NO. 40 | |
| pCLS27731 | SEQ ID | SEQ ID | SEQ ID | SEQ ID | SEQ ID | SEQ ID | 301-455 of SEQ ID NO. 5 |
| (SEQ ID NO. 208) | NO. 37 | NO. 39 | NO. 45 | NO. 38 | NO. 46 | NO. 40 | |
| pCLS27732 | SEQ ID | SEQ ID | SEQ ID | SEQ ID | SEQ ID | SEQ ID | 296-410 of SEQ ID NO. 12 |
| (SEQ ID NO. 209) | NO. 37 | NO. 39 | NO. 45 | NO. 38 | NO. 46 | NO. 40 | |
| pCLS27733 | SEQ ID | SEQ ID | SEQ ID | SEQ ID | SEQ ID | SEQ ID | 592-738 of SEQ ID NO. 19 |
| (SEQ ID NO. 210) | NO. 37 | NO. 39 | NO. 45 | NO. 38 | NO. 46 | NO. 40 | |

According to one embodiment, the N-CAR of the invention comprises at least one of any one of the polypeptide sequences described in the column “Amino acids of the inhibitory polypeptide used” in table 1.

According to one embodiment, the inhibitory signaling transduction domain of the N-CAR of the invention consists in one of any one of the polypeptide sequences from a receptor involved in transduction signal described in the column “Amino acids of the inhibitory polypeptide used” in table 1.

According to one embodiment, the intracellular domain of the N-CAR comprises a polypeptide sequence from Tumor-necrosis-factor related apoptosis inducing ligand (TRAIL) receptor.

Endogenous TRAIL is expressed as a 281-amino acid type II trans-membrane protein, which is anchored to the plasma membrane and presented on the cell surface. TRAIL was independently identified (Wiley S R, Schooley K Smolak P J, Din W S, Huang C P, Nicholl J K, et al. “Identification and characterization of a new member of the TN F family that induces apoptosis. Immunity 1995; 3:673-82; and Pitti R M, Marsters S A, Ruppert S, Donahue O, Moore A, Ashkenazi A, 1996.” Induction of apoptosis by Apo-2 ligand, a new member of the tumor necrosis factor cytokine family”. J Biol Chem; 271:12687-90.) In the first publication, sequence alignments indicated its close relation to other death ligands, with highest sequence similarities reported for Fas ligand (FasL).

TRAIL is expressed by natural killer cells, which, following the establishment of cell-cell contacts, can induce TRAIL-dependent apoptosis in target cells (Smyth M J, Cretney E, Takeda K, Wiltrout R H, Sedger L M, Kayagaki N, et al., 2001, “Tumor necrosis factor-related apoptosis-inducing ligand (TRAIL) contributes to interferon gamma-dependent natural killer cell protection from tumor metastasis. J Exp Med; 193:661-70).

Physiologically, the TRAIL-signaling system was shown to be essential for immune surveillance, for shaping the immune system through regulating T-helper cell 1 versus T-helper cell 2 as well as “helpless” CD8⁺ T-cell numbers, and for the suppression of spontaneous tumor formation (Janssen E M, Droin N M, Lemmens E E, Pinkoski M J, Bensinger Si, Ehst B D, et al. 2005, “CD4+ T-cell help controls CD8+ T-cell memory via TRAIL-mediated activation-induced cell death”. Nature; 434:88-93.).

As reviewed in the publication (Hellwig C T, and Rehm M, 2012 “TRAIL Signaling and Synergy Mechanisms Used in TRAIL-Based Combination Therapies”, Mol Cancer Ther. 11(1):3-13), TRAIL and agonistic antibodies raised against TRAIL death receptors are highly promising new anticancer agents. In this review is described the recent advances in the molecular understanding of TRAIL signaling and the progress made in using TRAIL or agonistic antibodies clinically in mono- and combination therapies. Human agonistic monoclonal antibodies targeting TRAIL-R1 (mapatumumab) or TRAIL-R2 (lexatumumab) were used to treat everal metastatic, triple (estrogen receptor, progesterone receptor, and HER2)-negative cancer cell lines (Malin D, Chen F, Schiller C, Koblinski J, Cryns V L. 2011″Enhanced metastasis suppression by targeting TRAIL receptor 2 in a murine model of triple-negative breast cancer.” Clin Cancer Res. 17(15):5005-15).

These publications, a disclose an chimeric antigen receptor comprising an extracellular domain specific for a TRAIL receptor Tthe present invention relates to an iCAR (or N CAR) comprising an intracellular signaling domain derived from a TRAIL receptor, and in particular from a TRAIL receptor selected from TR10D (other names: TNFRSF10D:DCR2, TRAILR4, TRUNDD), TR10A (TNF receptor superfamily member 10, TRAIL-R1 or CD261), or TR10B (TNF receptor superfamily member 10B, TRAIL-R2 or CD262).

The present invention relates to an iCAR (or N CAR) comprising an intracellular signaling domain derived from a TRAIL receptor, said TRAIL receptor is involved in in caspase-8-mediated apoptosis through proteolytic activation and further NF-kappa-B activation.

In one embodiment, the present invention provides an iCAR (or N CAR) comprising an intracellular signaling domain comprising at least one sequence selected from SEQ ID NO: 33 (human TR10D), SEQ ID NO: 34 (human TR10A) and SEQ ID NO: 35 (human TR10B); According to one embodiment, the intracellular domain of the N-CAR consists in one of any one of the polypeptide sequences selected from SEQ ID NO: 33 (human TR10D), SEQ ID NO: 34 (human TR10A) and SEQ ID NO: 35 (human TR10B);

In another embodiment the present invention provides an iCAR (or N CAR) comprising an intracellular signaling domain comprising at least one sequence having more than 80%, preferably 90% and more preferably 95% identity with SEQ ID NO: 33 (human TR10D), SEQ ID NO: 34 (human TR10A) and SEQ ID NO: 35 (human TR10B).

As preferred ones, said polypeptide sequences of receptor have more than 80%, preferably 90% and more preferably 95% identity with SEQ ID NO: 33 (human TR10D), SEQ ID NO: 34 (human TR10A) and SEQ ID NO: 35 (human TR10B). Human TR10D is also called TNFRSF10D, DCR2, TRAILR4 or TRUNDD and has as ORF names UNQ251/PRO288. Human TR10A is also called TNFRSF10A, APO2, DR4 or TRAILR1. Human TR10B is also called TNFRSF10B, DR5 KILLER, TRAILR2, TRICK2 or ZTNFR9 and has as ORF names UNQ160/PRO186.

According to another embodiment, the intracellular domain of the N-CAR comprises a polypeptide sequence from the CD200 receptor 1, more preferably a sequence comprising a sequence of SEQ ID NO:36.

In another embodiment the present invention provides an iCAR (or N CAR) comprising an intracellular signaling domain comprising at least one sequence having more than 80%, preferably 90% and more preferably 95% identity with SEQ ID NO:36. The cell surface glycoprotein CD200 receptor 1 (Uniprot ref: Q8TD46) represents another example of intracellular domain part of the iCAR (or N CAR) of the present invention. This inhibitory receptor for the CD200/OX2 cell surface glycoprotein limits inflammation by inhibiting the expression of proinflammatory molecules including TNF-alpha, interferons, and inducible nitric oxide synthase (iNOS) in response to selected stimuli (Wright G J, Cherwinski H, Foster-Cuevas M, Brooke G, Puklavec M J, Bigler M, Song Y, Jenmalm M, Gorman D, McClanahan T, Liu M R, Brown M H, Sedgwick J D, Phillips J H, Barclay A N. 2003 “Characterization of the CD200 receptor family in mice and humans and their interactions with CD200. J Immunol. >>171(6):3034-46).

As preferred one, said polypeptide sequences of receptor have more than 80%, preferably 90% and more preferably 95% identity with SEQ ID NO: 36 (human cell surface glycoprotein CD200 receptor 1).

The above sequences are listed in the following table 2.

TABLE 2
Amino-acid sequences of intracellular domain from a Tumor-necrosis-factor related apoptosis
inducing ligand (TRAIL) receptor and cell surface glycoprotein CD200 receptor 1

| | UniProt | SEQ | |
|---|---|---|---|
| Name | entry | ID NO: | Amino-acid sequence |
| TR10D_HUMAN | Q9UBN6 | 33 | MGLWGQSVPTASSARAGRYPGARTASGTRPWLLDPKILKFVVFIVAVLLPVRVDSATIPRQDEVPQ |
| | | | QTVAPQQQRRSLKEEECPAGSHRSEYTGACNPCTEGVDYTIASNNLPSCLLCTVCKSGQTNKSSCT |
| | | | TTRDTVCQCEKGSFQDKNSPEMCRTCRTGCPRGMVKVSNCTPRSDIKCKNESAASSTGKTPAAEET |
| | | | VTTILGMLASPYHYLIIIVVLVIILAVVVVGFSCRKKFISYLKGICSGGGGGPERVHRVLFRRRSC |
| | | | PSRVPGAEDNARNETLSNRYLQPTQVSEQEIQGQELAELTGVTVESPEEPQRLLEQAEAEGCQRRR |
| | | | ASATLEEGHAKETIQDQLVGSEKLFYEEDEAGSATSCLLLVPVNDADSADISTLLD |
| TR10A_HUMAN | O00220 | 34 | MAPPPARVHLGAFLAVTPNPGSAASGTEAAAATPSKVWGSSAGRIEPRGGGRGALPTSMGQHGPSA |
| | | | RARAGRAPGPRPAREASPRLRVHKTFKFVVVGVLLQVVPSSAATIKLHDQSIGTQQWEHSPLGELC |
| | | | PPGSHRSEHPGACNRCTEGVGYTNASNNLFACLPCTACKSDEEERSPCTTTRNTACQCKPGTFRND |
| | | | NSAEMCRKCSRGCPRGMVKVKDCTPWSDIECVHKESGNGHNIWVILVVTLVVPLLLVAVLIVCCCI |
| | | | GSGCGGDPKCMDRVCFWRLGLLRGPGAEDNAHNEILSNADSLSTFVSEQQMESQEPADLTGVTVQS |
| | | | PGEAQCLLGPAEAEGSQRRRLLVPANGADPTETLMLFFDKFANIVPFDSWDQLMRQLDLTKNEIDV |
| | | | VRAGTAGPGDALYAMLMKWVNKTGRNASIHTLLDALERMEERHAREKIQDLLVDSGKFIYLEDGTG |
| | | | SAVSLE |
| TR10B_HUMAN | O14763 | 35 | MEQRGQNAPAASGARKRHGPGPREARGARPGPRVPKTLVLVVAAVLLLVSAESALITQQDLAPQQR |
| | | | AAPQQKRSSPSEGLCPPGHHISEDGRDCISCKYGQDYSTHWNDLLFCLRCTRCDSGEVELSPCTTT |
| | | | RNTVCQCEEGTFREEDSPEMCRKCRTGCPRGMVKVGDCTPWSDIECVHKESGTKHSGEVPAVEETV |
| | | | TSSPGTPASPCSLSGIIIGVTVAAVVLIVAVFVCKSLLWKKVLPYLKGICSGGGGDPERVDRSSQR |
| | | | PGAEDNVLNEIVSILQPTQVPEQEMEVQEPAEPTGVNMLSPGESEHLLEPAEAERSQRRRLLVPAN |
| | | | EGDPTETLRQCFDDFADLVPFDSWEPLMRKLGLMDNEIKVAKAEAAGHRDTLYTMLIKWVNKTGRD |
| | | | ASVHTLLDALETLGERLAKQKIEDHLLSSGKFMYLEGNADSAMS |
| Cell surface | Q8TD46 | 36 | MLCPWRTANLGLLLILTIFLVAASSSLCMDEKQITQNYSKVLAEVNTSWPVKMATNAVLCCPPIAL |
| glycoprotein | | | RNLIIITWEIILRGQPSCTKAYRKETNETKETNCTDERITWVSRPDQNSDLQIRPVAITHDGYYRC |
| CD200 receptor | | | IMVTPDGNFHRGYHLQVLVTPEVTLFQNRNRTAVCKAVAGKPAAQISWIPEGDCATKQEYWSNGTV |
| 1_human | | | TVKSTCHWEVHNVSIVTCHVSHLTGNKSLYIELLPVPGAKKSAKLYIPYIILTIIILTIVGFIWLL |
| | | | KVNGCRKYKLNKTESTPVVEEDEMQPYASYTEKNNPLYDTTNKVKASEALQSEVDTDLHTL |

Extracellular Binding Domain or the N CAR According to the Invention

The inhibitory chimeric antigen receptor (iCAR or N-CAR) and the positive chimeric antigen receptor (P-CAR) according to the present invention comprise an extracellular ligand-binding domain.

The term “extracellular ligand-binding domain” as used herein is defined as an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. The combination of at least the two input signals corresponding to the recognition of different ligands by each extracellular domains of said N-CAR and P-CAR allows the inhibition of the P-CAR via the inhibitory transduction domain contained in the N-CAR.

The system of the invention aims to avoid the “off target” events, wherein the engineered immune cells target not only tumoral cells due in particularly to lack of specificity of the antigen (the latter being present on the cancerous cells but can also be present on normal cells).

Therefore, the extracellular binding domains within the scope of the invention are chosen in such a way that the one belonging to the P-CAR recognizes on-target cells (i.e. tumoral cells) and the one belonging to the N-CAR recognizes off-target cells (healthy cells). Thus, when the engineered immune cell encounters a cancerous cell, only the P-CAR is able to bind to it and not the N-CAR, and consequently the P-CAR can be activated and the cancerous cell killed. In the other issue, when the engineered immune cell encounters a normal cell, both P-CAR and N-CAR can bind to it, and consequently the N-CAR can inactivate the P-CAR: the normal cell will be preserved.

The antigen binding domain of the N-CAR can be any domain that binds to the off-tissue antigen including but not limited to a monoclonal antibody, a recombinant antibody, a human antibody, a humanized antibody, and a functional fragment thereof.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. No. 6,407,213, U.S. Pat. No. 5,766,886, International Publication No. WO 9317105, Tan et al., J. Immunol., 169: 1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8): 1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.).

In a preferred embodiment, said extracellular ligand-binding domain is a single chain antibody fragment (scFv). The latter comprises usually the light (V_{L}) and the heavy (V_{H}) variable fragment of a target antigen specific monoclonal antibody joined by a flexible linker. Other binding domain than scFv can also be used for predefined targeting of lymphocytes, such as camelid single-domain antibody fragments, receptor ligands like a vascular endothelial growth factor polypeptide, an integrin-binding peptide, heregulin or an IL-13 mutein, antibody binding domains, antibody hypervariable loops or CDRs as non-limiting examples.

In some embodiments, the antibody binding domain is a Fv, a Fab, a (Fab′)2, or a bi-functional (e.g. bi-specific) hybrid antibody (e.g., Lanzavecchia et al., Eur. J. Immunol. 17, 105 (1987)). In some embodiments, the antigen binding domain of the N-CAR of the invention binds an off-tissue antigen with wild-type or enhanced affinity.

By “affinity” is meant a measure of binding strength. Without being bound to theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes. Methods for calculating the affinity of an antibody for an antigen are known in the art, including use of binding experiments to calculate affinity. Antibody activity in functional assays (e.g., flow cytometry assay) is also reflective of antibody affinity. Antibodies and affinities can be phenotypically characterized and compared using functional assays (e.g, flow cytometry assay).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a SerGly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. The linker sequence may comprise amino acids glycine and serine. The linker sequence may comprise sets of glycine and serine repeats such as (Gly4Ser)n, where n is a positive integer equal to or greater than 1. The linker can be (Gly4Ser)4 or (Gly4Ser)3. Variation in the linker length may retain or enhance activity, giving rise to superior efficacy in activity studies.

In a preferred embodiment, the antigen binding domain of the N-CAR comprises an scFv. The off-tissue antigen recognized by the antigen binding domain of the N-CAR is preferably an antigen that is not present or present at low level on the tumour cells targeted by the P-CAR.

In a preferred embodiment, the antigen binding domain of the N-CAR comprises a scFv. The off-tissue antigen recognized by the antigen binding domain of the N-CAR is preferably an antigen that is not present or present at low level on the tumour cells targeted by the P-CAR and is expressed in normal tissue (non precancerous or non cancerous).

By “cancerous or tumor cell”, it is meant cells differing from normal cells in many ways that allow them to grow out of control and become invasive. Cancer cells are less specialized than normal cells and continue to divide without stopping. They are able to ignore signals that normally tell cells to stop dividing or that begin a process known as programmed cell death, or apoptosis, which the body uses to get rid of unneeded cells.

Present at low level on the tumour cells targeted by the P-CAR means that the expression of said off-tissue antigen is undetectable in tumor cells using any known technique of antigen detection (eg flow cytometry, himmunohisto chemistry, western blot) or represents less than 10% expression as compared to expression in a cell or a tissue used as a positive control.

In one embodiment said the antigen binding domain of the N-CAR comprises at least a scFv specific for any one of the following antigen CD56, CD205, CD83, CD206, CD200, CD36, RARRES1, Troponin C, Beta-1 integrin, CCKBR, GALR1, CD4, CD20, CD22, CD25, MUC1 antigen CD20.

In one embodiment said the antigen binding domain of the N-CAR comprises at least a scFv specific for any one of the following antigen CD56, CD205, CD83, CD206, CD200, CD36, or RARRES1.

In one embodiment said the antigen binding domain of the N-CAR comprises at least a scFv specific for any one of the following antigen Troponin C, Beta-1 integrin, CCKBR, or GALR1.

In one embodiment said the antigen binding domain of the N-CAR comprises a scFv specific for any one of the following antigen CD4, CD20, CD22, CD25, MUC1 antigen. In one embodiment said the antigen binding domain of the N-CAR comprises at least a scFv specific for any one of the following antigen CD20, PSMA, BCMA, CD19The below table 3 provides examples of combinations of N-CAR and P-CAR antigens. Combinations of a P-CAR directed to anti-D33, FLT3, MUC16, and to anti-MUC17 CAR with their N-CARs counterparts, are not part of the present invention.

| P-CAR Antigen | N-CAR Antigen |
|---|---|
| CD38 | CD56 antigen: expression on the surface of neurons, glia, skeletal muscle |
| | and natural killer cells |
| | CD205 antigen: expression on cortical thymic epithelial cells and by |
| | dendritic cell (DC) subsets |
| | CD83 antigen: expression on activated lymphocytes, Langerhans cells and |
| | interdigitating reticulum cells |
| | CD206 antigen: expression on the surface of macrophages and dendritic |
| | cells, on the surface of skin cells such as human dermal fibroblasts and |
| | keratinocytes |
| | CD200 antigen: expression on cells originating from the hematopoietic |
| | cells, activated T cells, endothelial neuronal cells and cells of the |
| | reproductive organs (ovaries and placental trophoblasts) |
| | CD36 antigen: expression in adipocytes endothelial cells and monocytes |
| | RARRES1 antigen: expression of this gene upregulated by tazarotene as |
| | well as by retinoic acid receptors |
| CS1 | Troponin C antigen: expression in heart |
| | Beta-1 integrin antigen: expression in endothelial cells and fibroblasts (at |
| | protein level). Expression in intestine, colon, testis, ovary, thymus, spleen |
| | and prostate |
| | CCKBR antigen: expression in stomach, pancreas, brain and gallbladder |
| | GALR1 antigen: expression in adrenal gland |
| | CUBN antigen: expression in kidney and small intestine |
| CD123 | CD4 antigen: expression in appendix, bone marrow, lymph node, tonsil and |
| | spleen |
| | CD20 antigen: expression mainly in spleen appendix and lymph node |
| | CD22 antigen: expression in particular in appendix, lymph node, tonsil and |
| | spleen |
| | CD25 antigen: expression mainly in bladder and lymph node |
| | MUC1 antigen: expression in kidney |
| ROR1 | Troponin C antigen: expression in heart |
| | Beta-1 integrin antigen: expression in endothelial cells and fibroblasts (at |
| | protein level). Expression in intestine, colon, testis, ovary, thymus, spleen |
| | and prostate |
| | CCKBR antigen: expression in stomach, pancreas, brain and gallbladder |
| | GALR1 antigen: expression in adrenal gland |
| | MUC1 antigen: expression in kidney |
| CD33 | Antigens specifically expressed in dendritic cells and/or haematopoetic stem |
| | cells such as ITGAX, CD1E, CD34, CD1C, CD123, CD141 |
| FLT3 | Antigens specifically expressed in haematopoetic stem cells such as CD34 or |
| | specifically expressed in Brain cerebellum such as ZP2, GABRA6, CRTAM, |
| | GRM4, MDGA1 |
| MSLN | Antigens specifically expressed in lung such as SFTPC, ROS1, SLC6A4, AGTR2 |
| MUC16 | Antigens specifically expressed in salivary gland such as LRRC26, HTR3A, |
| | TMEM211, MRGPRX3 |
| MUC17 | Antigens specifically expressed in colon & small intestine such as MEP1B, |
| | TMIGD1, CEACAM20, ALPI |
| CD20 | CD20 |

Extracellular-Binding Domain of N-CAR According to the Invention

In the various embodiments of the aspects delineated herein, the binding of an antigen to the NCAR activates the intracellular signaling domain resulting in a decrease in an immune response, preferably in the CTL activity.

In the present invention, the antigen binding domain of the N-CAR binds to a cell-surface protein present in normal tissue but not present or present at lower level on a tumor as compared to a the same cell in normal tissue said binding domain binds to an off-tissue antigen.

N-CAR antigens could also include antigens that are independent of the antigen that the P-CAR is targeting and that are down-regulated in tumor of interest, but present in all normal tissues of concern. Examples of such antigens for pancreatic ductal adenocarcinoma are TMPRSS11B, CYP17A1 and ATP4B and examples of such antigens for kidney clear cell carcinoma are GP2, MUC21, CLCA4 and SLC27A6.

In certain embodiments, the subject has metastatic breast cancer, hematological malignancy, or a solid tumor, and the human leukocyte antigen (HLA) is HLA-I. In certain embodiments, the subject has a tumor that has undergone epithelium to mesenchymal transition (EMT), and the antigen is one or more of an Epithelial-mesenchymal transition (EMT) antigen, E-cadherin, and cytokeratin. In various embodiments, the binding of the inhibitory chimeric antigen receptor and the antigen, decreases cell death in a cell comprising the antigen. The method can reduce graft versus host disease (GVHD) in the subject, or a symptom thereof.

Extracellular-Binding Domain of P-CAR

The extracellular ligand-binding domain of a P CAR according to the present invention can also comprise a peptide binding an antigen of the target, a peptide or a protein binding an antibody that binds an antigen of the target, a peptide or a protein ligand such as a growth factor, a cytokine or a hormone as non-limiting examples binding a receptor on the target, or a domain derived from a receptor such as a growth factor receptor, a cytokine receptor or a hormone receptor as non-limiting examples, binding a peptide or a protein ligand on the target. Preferably the target is a cell.

As non-limiting example, the ligand of the target can be a tumor-associated surface antigen, such as ErbB2 (HER2/neu), carcinoembryonic antigen (CEA), epithelial cell adhesion molecule (EpCAM), epidermal growth factor receptor (EGFR), CD19, CD20, CD30, CD40, disialoganglioside GD2, GD3, C-type lectin-like molecule-1 (CLL-1), ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-I, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the A1 domain of tenascin-C (TnC A1) and fibroblast associated protein (fap), LRP6, melamona-associated Chondroitin Sulfate Proteoglycan (MCSP), CD38/CS1, MART1, WT1, MUC1, LMP2, Idiotype, NY-ESO-1, Ras mutant, gp100, proteinase 3, bcr-abl, tyrosinase, hTERT, EphA2, ML-TAP, ERG, NA17, PAX3, ALK, Androgen receptor; a lineage-specific or tissue specific antigen such as CD3, CD4, CD8, CD24, CD25, CD34, CD79, CD116, CD117, CD135, CD123, CD133, CD138, CTLA-4, B7-1 (CD80), B7-2 (CD86), endoglin, a major histocompatibility complex (MHC) molecule, BCMA (CD269, TNFRSF 17), or a virus-specific surface antigen such as an HIV-specific antigen (such as HIV gp120); an EBV-specific antigen, a CMV-specific antigen, a HPV-specific antigen, a Lasse Virus-specific antigen, an Influenza Virus-specific antigen as well as any derivate or variant of these surface markers. In specific cases, the ligand that the chimeric antigen receptor recognizes is present on the surface of a target cell, particularly cancer cell or viral cell. In some embodiments, the ligand that the chimeric antigen receptor recognizes is present in a tumor microenvironment. In some aspects of the invention, the ligand that the chimeric antigen receptor recognizes is a growth factor.

In a preferred embodiment, CD33, BCMA and EGFRVIII do not belong to the present invention.

N-CAR Architecture & its Other Components

The N-CAR of the invention may have the single-chain or the multi-chain architecture. The multi-chain conformation is disclosed in WO2014039523.

The N-CAR of the present invention is a transmembrane polypeptide containing at least:
an extracellular binding domain;
a transmembrane domain and,
an intracellular domain comprising at least one polypeptide sequence involved in transduction signal, preferably an inhibitory transduction signal said polypeptide sequence is not significantly expressed in T-cell, and/or said polypeptide sequence is from a (TRAIL) receptor and/or from a CD200 receptor 1, provided that said polypeptide sequence is not a sequence selected from group consisting of SEQ ID NO:13 (human SIGL8), SEQ ID NO:14 (human SIGL7), SEQ ID NO:17 (human SIGL5), SEQ ID NO:20 (human SIGL9), SEQ ID NO: 21 (human SIGL6), SEQ ID NO:22 (human CD33), SEQ ID NO:26 (human SIG12), SEQ ID NO:31 (human SIG11), SEQ ID NO:32 (human SIG10) and SEQ ID NO:19 (human PECA1).

In a preferred embodiment, said intracellular domain comprises a polypeptide sequence also called inhibitory transduction domain. By “inhibitory transduction domain”, it is meant here a transmembrane polypeptide which contains a region encoding for an inhibitory transduction signal.

In a preferred embodiment said inhibitory transduction signal attenuates the activity of the immune cells, in particular of the CTL activity, preferably a CTL activity induced upon binding of a P-CAR of the invention.

According to a preferred embodiment, the N-CAR comprises at least:
an extracellular binding domain;
a transmembrane domain and,
an intracellular domain comprising at least one polypeptide sequence involved in transduction signal, preferably an inhibitory transduction signal said polypeptide sequence is not significantly expressed in T-cell, and/or said polypeptide sequence is from a (TRAIL) receptor and/or from a CD200 receptor 1, provided that said polypeptide sequence is not a sequence selected from group consisting of SEQ ID NO:13 (human SIGL8), SEQ ID NO:14 (human SIGL7), SEQ ID NO:17 (human SIGL5), SEQ ID NO:20 (human SIGL9), SEQ ID NO: 21 (human SIGL6), SEQ ID NO:22 (human CD33), SEQ ID NO:26 (human SIG12), SEQ ID NO:31 (human SIG11), SEQ ID NO:32 (human SIG10) and SEQ ID NO:19 (human PECA1)
wherein said inhibitory transmembrane polypeptide comprises a sequence with more than 80%, preferably 90% and more preferably 95% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

According to a more preferred embodiment, the N-CAR comprises at least:
an extracellular binding domain;
a transmembrane domain and,

an intracellular domain comprising
an inhibitory transmembrane polypeptide comprising a sequence selected from the list consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

According to an embodiment, the N-CAR comprises at least: an extracellular binding domain;
a transmembrane domain and,
an intracellular domain comprising
an inhibitory transmembrane polypeptide comprising a sequence with more than 80%, preferably 90% and more preferably 95% identity with SEQ ID NO:36 (CD200 receptor 1).

According to a more preferred embodiment, the N-CAR comprises at least:
an extracellular binding domain;
a transmembrane domain and,
an intracellular domain comprising
a sequence of SEQ ID NO:36 (CD200 receptor 1).

According to a preferred embodiment, the N-CAR comprises at least:
an extracellular binding domain;
a transmembrane domain and,
an intracellular domain comprising a sequence with more than 80%, preferably 90% and more preferably 95% identity with SEQ ID NO: 33 (human TR10D), SEQ ID NO: 34 (human TR10A) or SEQ ID NO: 35 (human TR10B).

According to an even more preferred embodiment, the N-CAR comprises at least:
an extracellular binding domain;
a transmembrane domain and,
an intracellular domain comprising a sequence selected from the list consisting of

SEQ ID NO: 33 (human TR10D), SEQ ID NO: 34 (human TR10A) and SEQ ID NO: 35 (human TR10B).

According to one more preferred embodiment, the N-CAR comprises at least:
an extracellular binding domain;
a transmembrane domain and,
an intracellular domain comprising a sequence with more than 80%, preferably 90% and more preferably 95% identity with SEQ ID NO: 33 (human TR10D).

According to one more preferred embodiment, the N-CAR comprises at least:
an extracellular binding domain;
a transmembrane domain and,
an intracellular domain comprising a sequence of SEQ ID NO: 33 (human TR10D).

According to one more preferred embodiment, the N-CAR comprises at least:
an extracellular binding domain;
a transmembrane domain and,
an intracellular domain comprising a sequence with more than 80%, preferably 90% and more preferably 95% identity with SEQ ID NO: 34 (human TR10A).

According to one more preferred embodiment, the N-CAR comprises at least:
an extracellular binding domain;
a transmembrane domain and,
an intracellular domain comprising a sequence of SEQ ID NO: 34 (human TR10A).

According to one more preferred embodiment, the N-CAR comprises at least:
an extracellular binding domain;
a transmembrane domain and,
an intracellular domain comprising a sequence with more than 80%, preferably 90% and more preferably 95% identity with SEQ ID NO: 35 (human TR10B).

According to one more preferred embodiment, the N-CAR comprises at least:
an extracellular binding domain;
a transmembrane domain and,
an intracellular domain comprising a sequence of SEQ ID NO: 35 (human TR10B).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of aminoacids No 201-375 from SEQ ID NO:8 (human KI2LA).

By “polypeptide sequence consisting essentially of”, it is meant that the polypeptide is the one identical to the part of the inhibitory molecule which is used in the N-CARs presented here. However, at least one to a few amino acid substitution(s) is(are) contemplated within the present invention in order to bring a modulation of its inhibitory function in case of need.

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 206-348 from SEQ ID NO:2 (human KIR2DL1).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 206-348 from SEQ ID NO:1 (human KIR2DL2).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 206-341 from SEQ ID NO:4 (human KIR2DL3).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 206-348 from SEQ ID NO:2 (human KIR2DL1).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 203-377 from SEQ ID NO:6 (human KIR2DL4).

According to one preferred embodiment, the N-CAR from the present comprising:
an extracellular domain comprising an antigen binding domain;
a transmembrane domain;
an intracellular domain;

wherein said intracellular domain comprises a polypeptide sequence consisting essentially of amino acids No 301-444 from SEQ ID NO:7 (human KIR3DL1).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 301-455 from SEQ ID NO:5 (human KIR3DL2).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 204-310 from SEQ ID NO:24 (human FRGR2B).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 214-343 from SEQ ID NO:9 (human MILR1).

According to one preferred embodiment, the N-CAR of the present comprising:
an extracellular domain comprising an antigen binding domain;
a transmembrane domain;
an intracellular domain;
wherein said polypeptide sequence of the receptor of amino acids No 216-448 from SEQ ID NO:10 (human LIRB4).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 420-631 from SEQ ID NO:11 (human LIRB3).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 296-410 from SEQ ID NO:12 (human KI3L3).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 419-590 from SEQ ID NO:15 (human LIRB5).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 420-598 from SEQ ID NO:16 (human LIRB2).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 375-515 from SEQ ID NO:18 (human FCRL4).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 753-977 from SEQ ID NO:23 (human FCRL5).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 388-508 from SEQ ID NO:24 (human FCRL2).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 292-429 from SEQ ID NO: 25 (human FCRL1).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 564-734 from SEQ ID NO:27 (human FCRL3).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 147-269 from SEQ ID NO:28 (human MPZL1).

According to one preferred embodiment, the N-CAR of the present comprising:
an extracellular domain comprising an antigen binding domain;
a transmembrane domain;
an intracellular domain;
wherein said intracellular domain comprises a polypeptide sequence consisting essentially of amino acids No 151-303 from SEQ ID NO:29 (human PILRA).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 329-417 from SEQ ID NO:30 (human PVR).

According to one preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 229-325 from SEQ ID NO:36 (human CD200 receptor1).

According to a preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 181-386 from SEQ ID NO:33 (human TR10D).

According to a preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 230-468 from SEQ ID NO:34 (human TR10A)

According to a preferred embodiment, the N-CAR of the present comprising:
an extracellular domain comprising an antigen binding domain;
a transmembrane domain;
an intracellular domain;

wherein said intracellular domain comprises a polypeptide sequence consisting essentially of amino acids No 179-440 from SEQ ID NO:35 (human TR10B).

According to another preferred embodiment, the N-CAR comprises at least:
an extracellular binding domain;
an transmembrane domain, and;
a linker between the extracellular binding domain and the transmembrane domain,

said linker can be any one known by the skilled man in the art.

Preferably, this linker is a GS linker 1 or a GS linker 2 comprising a sequences of SEQ ID NO:39 and SEQ ID NO:40, more preferably this linker is a GS linker 1 or a GS linker 2 consisting in a sequences of SEQ ID NO:39 and SEQ ID NO:40.

Therefore, the extracellular part of the N-CAR may comprises:
an extracellular-binding domain comprising at least one scFvs from a monoclonal antibody for binding to “off-target” antigen expressed on healthy cells; and preferably said off-target” antigen is not expressed on cells targeted by the P-CAR.a transmembrane domain and;
a linker binding together the two previous components.

Said above scFvs of a monoclonal antibody binds preferably to “off-target” antigens expressed in healthy tissues or healthy cells.

For instance, for the treatment of acute myeloid leukemia (AML), when the antigen targeted by the P-CAR is CD123, said extracellular-binding domain of the N-CAR binds to at least one “off-target” antigen expressed on healthy cells or healthy immune cells that may be chosen amongst an antigen selected from CD4 antigen (expressed in appendix, bone marrow, lymph node, tonsil and spleen), CD20 antigen (expressed mainly in spleen appendix and lymph node), CD22 antigen (expressed in particular in appendix, lymph node, tonsil and spleen), CD25 antigen (expressed mainly in bladder and lymph node) and MUC1 antigen (expressed in kidney).

Therefore, in one embodiment, the N-CAR of the invention comprises at least:
an extracellular domain comprising at least one scFv from and an antibody binding specifically to an antigen selected from CD4 antigen, CD20 antigen, CD22 antigen, CD25 antigen and/or MUC1 antigen; or a combination thereof.
a linker with a SEQ ID NO:38, SEQ ID NO:39 or SEQ ID NO:40; and,
a transmembrane domain
an intracellular domain comprising a sequence selected from the list consisting of SEQ ID NO: 33 (human TR10D), SEQ ID NO: 34 (human TR10A) and SEQ ID NO: 35 (human TR10B).

In another embodiment, the N-CAR of the invention comprises at least:
an extracellular domain comprising an at least one scFv from an antibody binding specifically to an antigen selected from CD4 antigen, CD20 antigen, CD22 antigen, CD25 antigen and/or MUC1 antigen;
a linker with a SEQ ID NO:38, SEQ ID NO:39 or SEQ ID NO:40; and,
an intracellular domain comprising a sequence with more than 80%, preferably 90% and more preferably 95% identity with SEQ ID NO: 33 (human TR10D), SEQ ID NO: 34 (human TR10A) and SEQ ID NO: 35 (human TR10B).

In another embodiment, the N-CAR of the invention comprises at least:
an extracellular domain comprising at least one scFv from an antibody binding specifically to an antigen selected from CD4 antigen, CD20 antigen, CD22 antigen, CD25 antigen and/or MUC1 antigen;
a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from CD4 antigen, CD20 antigen, CD22 antigen, CD25 antigen and/or MUC1 antigen;
a linker with a SEQ ID NO:38, SEQ ID NO:39 or SEQ ID NO:40; and,
a transmembrane domain,
an intracellular domain comprising a sequence selected from the list consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from CD4 antigen, CD20 antigen, CD22 antigen, CD25 antigen and/or MUC1 antigen;
a linker with a SEQ ID NO:38, SEQ ID NO:39 or SEQ ID NO:40; and,
an intracellular domain comprising a sequence of SEQ ID NO:36 (CD200 receptor 1).

In another embodiment, N-CAR of the invention comprises at least:
an extracellular domain comprising at least one scFv from an antibody binding specifically to an antigen selected from CD4 antigen, CD20 antigen, CD22 antigen, CD25 antigen and/or MUC1 antigen-a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO:36 (CD200 receptor 1).

For instance for the treatment of multiple myeloma (MM), when the antigen targeted by the P-CAR is CD38, said extra-binding domain of the N-CAR binds to at least one “off-target” antigen expressed on healthy cells may be chosen amongst an antigen selected from CD56 antigen (expressed on the surface of neurons, glia, skeletal muscle and natural killer cells), CD205 antigen (expressed on cortical thymic epithelial cells and by dendritic cell (DC) subsets), CD83 antigen (expressed on activated lymphocytes, Langerhans cells and interdigitating reticulum cells), CD206 antigen (expressed on the surface of macrophages and dendritic cells, on the surface of skin cells such as human dermal fibroblasts and keratinocytes); CD200 antigen (expression on cells originating from the hematopoietic cells, activated T cells, endothelial neuronal cells and cells of the reproductive organs-ovaries and placental trophoblasts-); CD36 antigen (expressed in adipocytes endothelial cells and monocytes); RARRES1 antigen (expressed of this gene upregulated by tazarotene as well as by retinoic acid receptors)

For instance for the treatment of multiple myeloma (MM), when the antigen targeted by the P-CAR is CD22, said extra-binding domain of the N-CAR binds to at least one “off-target” antigen expressed on healthy cells may be chosen amongst an antigen selected from CD56 antigen (expressed on the surface of neurons, glia, skeletal muscle and natural killer cells), CD205 antigen (expressed on cortical thymic epithelial cells and by dendritic cell (DC) subsets), CD83 antigen (expressed on activated lymphocytes, Langerhans cells and interdigitating reticulum cells), CD206 antigen (expressed on the surface of macrophages and dendritic cells, on the surface of skin cells such as human dermal fibroblasts and keratinocytes); CD200 antigen (expression on cells originating from the hematopoietic cells, activated T cells, endothelial neuronal cells and cells of the reproductive organs-ovaries and placental trophoblasts-); CD36 antigen (expressed in adipocytes endothelial cells and monocytes); RARRES1 antigen (expressed of this gene upregulated by tazarotene as well as by retinoic acid receptors)

Therefore, in one embodiment, the N-CARs of the invention comprises at least:
an extracellular domain comprising at least one scFv from an antibody binding specifically to an antigen selected from CD56 antigen, CD205 antigen, CD83 antigen, CD206 antigen; CD200 antigen; CD36 antigen and/or RARRES1 antigen,
a linker with a SEQ ID NO:38, SEQ ID NO:39 or SEQ ID NO:40; and,
an intracellular domain comprising a sequence with more than 80%, preferably 90% and more preferably 95% identity with SEQ ID NO: 33 (human TR10D), SEQ ID NO: 34 (human TR10A) and SEQ ID NO: 35 (human TR10B).

In one embodiment, the N-CAR of the invention comprises at least:
an extracellular domain comprising at least one scFv from an antibody binding specifically to an antigen selected from CD56 antigen, CD205 antigen, CD83 antigen, CD206 antigen; CD200 antigen; CD36 antigen and/or RARRES1 antigen;
a linker with a SEQ ID NO:38, SEQ ID NO:39 or SEQ ID NO:40; and,
an intracellular domain comprising a sequence selected from the list consisting of SEQ ID NO: 33 (human TR10D), SEQ ID NO: 34 (human TR10A) or SEQ ID NO: 35 (human TR10B),

In another embodiment, the N-CAR of the invention comprises at least:
an extracellular domain comprising at least one scFv from an antibody binding specifically to an antigen selected from CD56 antigen, CD205 antigen, CD83 antigen, CD206 antigen; CD200 antigen; CD36 antigen and/or RARRES1 antigen;
a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO: 1 (human KI2L2), SEQ ID NO: 2 human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from CD56 antigen, CD205 antigen, CD83 antigen, CD206 antigen; CD200 antigen; CD36 antigen and/or RARRES1 antigen; —a linker with a SEQ ID NO:38, SEQ ID NO:39 or SEQ ID NO:40; and,
an intracellular domain comprising a sequence selected from the list consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from CD56 antigen, CD205 antigen, CD83 antigen, CD206 antigen; CD200 antigen; CD36 antigen and/or RARRES1 antigen;
a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO:36 (CD200 receptor 1).

In another embodiment, the N-CAR of the invention comprises at least:
an extracellular domain comprising at least one scFv from antibodies binding specifically to an antigen selected from CD56 antigen, CD205 antigen, CD83 antigen, CD206 antigen; CD200 antigen; CD36 antigen and/or RARRES1 antigen;
a linker with a SEQ ID NO:38, SEQ ID NO:39 or SEQ ID NO:40; and,
an intracellular domain comprising a sequence of SEQ ID NO:36 (CD200 receptor 1).

For instance for the treatment of chronic lymphocytic leukemia (CLL), when the antigen targeted by the P-CAR is CS1, said extra-binding domain of the N-CAR binds to “off-target” antigens expressed on healthy cells that may be chosen amongst troponin C antigen (expressed in heart); beta-1 integrin antigen (expressed in endothelial cells and fibroblasts, intestine, colon, testis, ovary, thymus, spleen and prostate); CCKBR antigen (expression in stomach, pancreas, brain and gallbladder); GALR1 antigen (expressed in adrenal gland); and CUBN antigen (expressed in kidney and small intestine).

Therefore, in one embodiment, the N-CAR of the invention may comprise at least:
an extracellular domain comprising at least one scFv from an antibody binding specifically to an antigen selected from troponin C antigen; beta-1 integrin antigen; CCKBR antigen; GALR1 antigen and CUBN antigen;
a linker with a SEQ ID NO:38, SEQ ID NO:39 or SEQ ID NO:40; and,
an intracellular domain comprising a sequence with more than 80%, preferably 90% and more preferably 95% identity with SEQ ID NO: 33 (human TR10D), SEQ ID NO: 34 (human TR10A) or SEQ ID NO: 35 (human TR10B).

In one embodiment, the N-CAR of the invention comprises at least:
an extracellular domain comprising at least one scFv from an antibody binding specifically to an antigen selected from troponin C antigen beta-1 integrin antigen; CCKBR antigen; GALR1 antigen and/or CUBN antigen;
a linker with a SEQ ID NO:38, SEQ ID NO:39 or SEQ ID NO:40; and,
an intracellular domain comprising a sequence selected from the list consisting of SEQ ID NO: 33 (human TR10D), SEQ ID NO: 34 (human TR10A) or SEQ ID NO: 35 (human TR10B).

In another embodiment, the N-CAR of the invention comprises at least:
an extracellular domain comprising at least one scFv from an antibody binding specifically to an antigen selected from troponin C antigen beta-1 integrin antigen; CCKBR antigen; GALR1 antigen and/or CUBN antigen;
a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO: 1 (human KI2L2), SEQ ID NO: 2 human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from troponin C antigen, beta-1 integrin antigen; CCKBR antigen; GALR1 antigen and/or CUBN antigen;
a linker with a SEQ ID NO:38, SEQ ID NO:39 or SEQ ID NO:40; and,
an intracellular domain comprising a sequence selected from the list consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from troponin C antigen, beta-1 integrin antigen; CCKBR antigen; GALR1 antigen and/or CUBN antigen;
a linker with a SEQ ID NO:38, SEQ ID NO:39 or SEQ ID NO:40; and,
an intracellular domain comprising a sequence with more than 80%, preferably 90% and more preferably 95% identity with SEQ ID NO:36 (CD200 receptor 1).

In another embodiment, the N-CAR of the invention comprises at least:
an extracellular domain comprising at least one scFv from an antibody binding specifically to an antigen selected from troponin C antigen (; beta-1 integrin antigen; CCKBR antigen; GALR1 antigen and/or CUBN antigen;
a linker with a SEQ ID NO:38, SEQ ID NO:39 or SEQ ID NO:40; and,
an intracellular domain comprising a sequence of SEQ ID NO:36 (CD200 receptor 1).

For instance for the treatment of chronic lymphocytic leukemia (CLL) or a solid tumor, when the antigen targeted by the P-CAR is ROR1, said extra-binding domain of the N-CAR binds to at least one “off-target” antigen expressed on healthy cells selected from the list consisting of troponin C antigen (expressed in heart); beta-1 integrin antigen (expressed in endothelial cells and fibroblasts, intestine, colon, testis, ovary, thymus, spleen and prostate); CCKBR antigen (expression in stomach, pancreas, brain and gallbladder); GALR1 antigen (expressed in adrenal gland) or MUC1 antigen (expressed in kidney).

Therefore, in one embodiment, the N-CAR of the invention comprises at least:
an extracellular domain comprising at least one scFv from an antibody binding specifically to an antigen selected from troponin C antigen (; beta-1 integrin antigen; CCKBR antigen; GALR1 antigen and/or MUC antigen;
a linker with a SEQ ID NO:38, SEQ ID NO:39 or SEQ ID NO:40; and,
an intracellular domain comprising a sequence with more than 80%, preferably 90% and more preferably 95% identity with SEQ ID NO: 33 (human TR10D), SEQ ID NO: 34 (human TR10A) and SEQ ID NO: 35 (human TR10B).

In one embodiment, the N-CAR of the invention comprises at least:
an extracellular domain comprising at least one scFv from an antibody binding specifically to an antigen selected from troponin C antigen (; beta-1 integrin antigen; CCKBR antigen; GALR1 antigen and/or MUC antigen;
a linker with a SEQ ID NO:38, SEQ ID NO:39 or SEQ ID NO:40; and,
an intracellular domain comprising a sequence selected from the list consisting of SEQ ID NO: 33 (human TR10D), SEQ ID NO: 34 (human TR10A) or SEQ ID NO: 35 (human TR10B)

In another embodiment, N-CARs of the invention may comprise at least:
an extracellular domain comprising at least one scFv from an antibody binding specifically to an antigen selected from troponin C antigen (; beta-1 integrin antigen; CCKBR antigen; GALR1 antigen and/or MUC antigen;
a linker with a SEQ ID NO:38, SEQ ID NO:39 or SEQ ID NO:40; and,
an intracellular domain comprising a sequence with more than more than 80%, preferably 90% and more preferably 95% identity with SEQ ID NO: 1 (human KI2L2), SEQ ID NO: 2 human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from troponin C antigen (; beta-1 integrin antigen; CCKBR antigen; GALR1 antigen and/or MUC antigen;
a linker with a SEQ ID NO:38, SEQ ID NO:39 or SEQ ID NO:40; and,
an intracellular domain comprising a sequence selected from the list consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR).

In another embodiment, the N-CAR of the invention comprises at least:
an extracellular domain comprising at least one scFv from an antibody binding specifically to an antigen selected from troponin C antigen (; beta-1 integrin antigen; CCKBR antigen; GALR1 antigen and/or MUC antigen.

In other embodiments, the N-CAR of the present invention is a transmembrane polypeptide containing at least:
an extracellular binding domain;
an intracellular domain comprising an inhibitory transduction domain,

wherein said inhibitory transduction domain of intracellular domain is used alone, fused to a separately chosen transmembrane domain, optionally, the latter being fused to the extracellular binding domain by a hinge.

Transmembrane Domain

In one embodiment, the transmembrane domain comprises the transmembrane region(s) of the alpha, beta or zeta chain of the T-cell receptor, PD-1, 4-16B, OX40, ICOS, CTLA-4, LAG3, 2B4, BTLA4, TIM-3, TIGIT, SIRPA, CD28, CD3 epsilon, CD45, CD4, CD5, CD8, CD9, CD16, CD22, CD33, CD37, CD64, CD80, CD86, CD134, CD137 or CD154.

The distinguishing features of appropriate transmembrane domains comprise the ability to be expressed at the surface of a cell, preferably in the present invention an immune cell, in particular lymphocyte cells or Natural killer (NK) cells, and to interact together for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a subunit of the T cell receptor such as α, β, [symbol] or [symbol] , polypeptide constituting CD3 complex, IL2 receptor p55 (α chain), p75 (β chain) or [symbol] chain, subunit chain of Fc receptors, in particular Fc [symbol] receptor III or CD proteins. Alternatively the transmembrane domain can be synthetic and can comprise predominantly hydrophobic residues such as leucine and valine. In a preferred embodiment said transmembrane domain is derived from the human CD8 alpha chain (e.g. NP_001139345.1). Said transmembrane domain can also be a CD8 transmembrane domain (alpha and beta chains). Said Transmembrane domain can be engineered to create obligated hetero or homodimers. In particular embodiment said CARs can comprise transmembrane domains or intracellular domains which can only dimerize after ligand recognition. Another example of transmembrane domain can be NKG2-D receptor. NKG2D (natural killer cell group 2D) is a C-type lectin-like receptor expressed on NK cells, γδ-TcR⁺ T cells, and CD8⁺αβ-TcR⁺ T cells (Bauer, Groh et al., 1999, Science 285(5428):727-9. NKG2D is associated with the transmembrane adapter protein DAP10 (Wu, Song et al. 1999, Science 285(5428):730-2), whose cytoplasmic domain binds to the p 85 subunit of the PI-3 kinase.

Another example of transmembrane domain can be a receptor tyrosine kinase. Receptor tyrosine kinase are cell surface receptors involved in different critical cellular regulatory process including cell proliferation, cell differentiation, cell survival, cell migration, as well as cell cycle control. Receptor tyrosine kinase comprises an extracellular domain, a single transmembrane helix and an intracellular domain comprising tyrosine kinase function that is most of time autoregulated by additional carboxy-terminal and juxtamembrane domains. Activation of receptor tyrosine kinase is generally elicited by ligand-mediated dimerization. Thanks to their bivalence, growth hormone ligand has the capacity to simultaneously interact with two receptor monomers and promotes dimerization. Such dimerization induces the activation of intracellular kinase domains through conformational changes followed by trans-phosphorylation of different tyrosines located within their intracellular domain. The different phosphotyrosines generated eventually serve as docking site for the recruitment of downstream signaling partners that activate the cellular regulatory pathways. Said CAR can comprise the extracellular domain, transmembrane, and/or the intracellular domain of a receptor tyrosine kinase, preferably selected from the group consisting of TrkA, c-Kit, FGFR and EGFR/Erb. Said tyrosine kinase transmembrane domain and/or intracellular domain can be linked to an extracellular ligand binding domain and intracellular domain according to the present invention. Said engineered cells may comprise different N- and P-CAR comprising different transmembrane domains.

Said transmembrane domain can also be an integrin. Integrins are heterodimeric integral membrane proteins composed of a [symbol] and [symbol] [symbol] chains which combined together form the LFA-1 (integrin lymphocyte function-associated antigen-1) which is expressed on all leukocytes. LFA-1 plays a central role in leukocyte intercellular adhesion through interactions with its ligand, ICAMs 1-3 (intercellular adhesion molecules 1 through 3), and also it has an important role in lymphocyte co-stimulatory signaling (Chen and Flies 2013, Nat Rev Immunol 13(4):227-42). The molecular details of the binding of LAF-1 to its immunoglobulin ICAM-1 are quite known allowing a careful engineering of LAF-1 binding site. The affinity of [symbol] _{L} domain for ICAM-1 is regulated by the displacement of its C-terminal helix which is conformational linked to alterations of specific loops in LAF-1. The active and low conformations differ of 500 and 10,000 folds. It is also interesting to note that two types of antagonists are known for LFA-1 and their mechanism of action is known. Integrin cell surface adhesion receptors can transmit a signal from the outside to inside but also vice-versa. There are cytoskeletal proteins as Talin which binds to the integrin tail LFA-1 to transfer a message from inside to outside.

According to one embodiment, the transmembrane domain comprises the transmembrane region of PD-1 or the transmembrane region(s) of CD8 alpha.

According to one preferred embodiment, the transmembrane domain comprises the transmembrane region of CD8 alpha. In one aspect of the invention, the transmembrane domain is attached to the extracellular domain of the N-CAR via a hinge.

Hinge

In a preferred embodiment, in the hinge of the N-CAR is a human immunoglobulin hinge.

In a more preferred embodiment, the hinge of the N-CAR is an IgG1 hinge or a CD8 alpha hinge.

The term “stalk region” (also named hinge region) used herein generally means any oligo- or polypeptide that functions to link the transmembrane domain to the extracellular ligand-binding domain. In particular, stalk region are used to provide more flexibility and accessibility for the extracellular ligand-binding domain. A stalk region may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, CD28 or RTK, or from all or part of an antibody constant region. Alternatively the stalk region may be a synthetic sequence that corresponds to a naturally occurring stalk sequence, or may be an entirely synthetic stalk sequence.

The present invention encompasses a recombinant DNA construct comprising sequences encoding an N-CAR as defined above, wherein the N-CAR comprises an extracellular domain such as an antibody fragment that binds specifically to an off-tumor antigen, and wherein the sequence of the extracellular domain is contiguous with and in the same reading frame as a nucleic acid sequence encoding a transmembrane domain and an intracellular domain. An exemplary N-CAR construct may comprise an optional leader sequence, an extracellular off-tissue antigen binding domain, a hinge, a transmembrane domain, and an intracellular inhibitory signaling domain.

According to one preferred embodiment, a hinge according to the invention comprises the a sequence from IgG1 or from CD8 alpha, preferably of SEQ ID NO. 51 and 50.

Engineered Immune Cells

In one aspect the present invention provides an immune cell comprising at least one N-CAR according to the invention (as described above).

In one aspect the present invention provides an immune cell comprising at least one N-CAR according to the invention (as described above) and at least one P-CAR, according to the invention.

In one aspect of the invention, an isolated immune cell comprises a P-CAR comprising:
an extracellular domain comprising an antigen binding domain;
a transmembrane domain;
an intracellular domain;

and an N-CAR as described previously.

The present invention encompasses an immune cell comprising a single chain (sc) or a multi chain (mc) N-CAR and a sc P-CAR.

The present invention encompasses an immune cell comprising a single chain (sc) or a multi chain (mc) N-CAR and a mc P-CAR.

The present invention encompasses an immune cell comprising a single chain (sc) N-CAR and a sc P-CAR.

The present invention encompasses an immune cell comprising a multi chain (mc) N-CAR and a sc P-CAR.

The present invention encompasses an immune cell comprising a multi chain (mc) N-CAR and a mc P-CAR. The present invention also relates to isolated cells or cell lines susceptible to be obtained by said method to engineer cells.

The present invention also relates to isolated cells or cell lines susceptible to be obtained by a method to engineer cells according to the present invention.

Said immune cell refers to a cell of hematopoietic origin functionally involved in the initiation and/or execution of innate and/or adaptive immune response. Said immune cell according to the present invention can be derived from a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. Said isolated cell can also be a dendritic cell, killer dendritic cell, a mast cell, a NK-cell, a B-cell or a T cell. Said isolated cell may comprise a population of N-CARs and CARs each one comprising different extracellular ligand binding domains. In particular, said isolated cell comprises exogenous polynucleotide sequence encoding N-CAR and P-CAR.

In one preferred embodiment, said isolated cell comprising at least one N-CAR and one CAR as described above is a T-cell.

In a more preferred embodiment, said isolated cell comprising at least one N-CAR and one CAR as described above is a human T-cell.

In a preferred embodiment, isolated immune cell is selected from the group consisting of inflammatory T-lymphocytes, cytotoxic T-lymphocytes, regulatory T-lymphocytes or helper T-lymphocytes.

Said cell may be derived from the group consisting of CD4+ T-lymphocytes and CD8+ T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art, may be used.

In one embodiment, said isolated immune cells are recovered from a healthy donor.

In another embodiment, said isolated immune cells are recovered from a patient diagnosed with cancer or from a patient diagnosed with an infection.

Said cells may part of a mixed population of cells which present different phenotypic characteristics. In the scope of the present invention is also encompassed a cell line obtained from a transformed T− cell according to the method previously described.

According to one embodiment, the antigen to which the antigen binding domain of the P-CAR binds is CD38 and the antigen to which the antigen binding domain of the N-CAR binds is an antigen selected from the list consisting of CD56, CD205, CD83, CD206, CD200 and CD36.

According to one embodiment, the antigen to which the antigen binding domain of the P-CAR binds is CD19 and the antigen to which the antigen binding domain of the N-CAR binds is an antigen selected from the list consisting of CD56, CD205, CD83, CD206, CD200 and CD36.

According to one embodiment, the antigen to which the antigen binding domain of the P-CAR binds is CD20 and the antigen to which the antigen binding domain of the N-CAR binds is an antigen selected from the list consisting of CD56, CD205, CD83, CD206, CD200 and CD36.

According to one embodiment, the antigen to which the antigen binding domain of the P-CAR binds is PCMA and the antigen to which the antigen binding domain of the N-CAR binds is an antigen selected from the list consisting of CD56, CD205, CD83, CD206, CD200 and CD36.

According to another embodiment, the antigen binding domain of the P-CAR binds is CS1 and the antigen to which the antigen binding domain of the N-CAR binds is an antigen selected from the list consisting of troponin C, beta-1 integrin, CCKBR, GALR1 or CUBN.

According to another embodiment, the antigen to which the antigen binding domain of the P-CAR binds is CD123 and the antigen to which the antigen binding domain of the N-CAR binds is an antigen selected from the list consisting of CD4, CD20, CD22, CD25 or MUC1.

According to another embodiment, the antigen to which the antigen binding domain of the P-CAR binds is ROR1 and the antigen to which the antigen binding domain of the N-CAR binds is an antigen selected from the list consisting of troponin C, beta-1 integrin, CCKBR, GALR1 or MUC1.

Positive Chimeric Antigen Receptor (P-CAR)

The present invention relates to “logical NOT” gates that involve, beside the above described N-CAR, at least one P-CAR which enable the engineered immune cell to trigger the destruction of tumoral targeted cells.

The P-CAR used within the scope of the invention can be a single-chain or a multi-chain CAR.

In one embodiment, the P-CAR is a single CAR; it comprises one transmembrane polypeptide comprising at least one extracellular ligand-binding domain and one extracellular domain comprising a signal-transducing domain.

In some embodiments, the immune cell comprises a multi-chain P-CAR as defined in WO2014/039523 which is incorporated herein by reference in its entirety

By multi-chain CAR is meant a CAR structure that comprises different polypeptides such as at least (1) a transmembrane polypeptide which comprises at least one extracellular ligand binding domain; and (2) a transmembrane polypeptide comprising at least one transduction domain such that said at least two polypeptides assemble together to form a functional multi-chain Chimeric Antigen Receptor (WO2014039523).

In another embodiment, this P-CAR is a multichain CAR such as described in WO2014039523, it comprises at least:
one transmembrane polypeptide comprising at least one extracellular ligand-binding domain and;
one transmembrane polypeptide comprising at least one signal-transducing domain.

For instance, said multi-chain CAR can comprise at least two of the following components:

a) one polypeptide comprising the transmembrembrane domain of FcsRI alpha chain fused to an extracellular ligand-binding domain,

b) one polypeptide comprising a part of N- and C-terminal cytoplasmic tail fused to the transmembrane domain of a FcRI beta chain, and/or

c) two additional polypeptides comprising each one part of an intracytoplasmic tail and/or the transmembrane domain of FcRI gamma chain,

whereby these different polypeptides multimerize together spontaneously to form dimeric, trimeric or tetrameric CARs.

In a preferred embodiment said chain are not covalently linked.

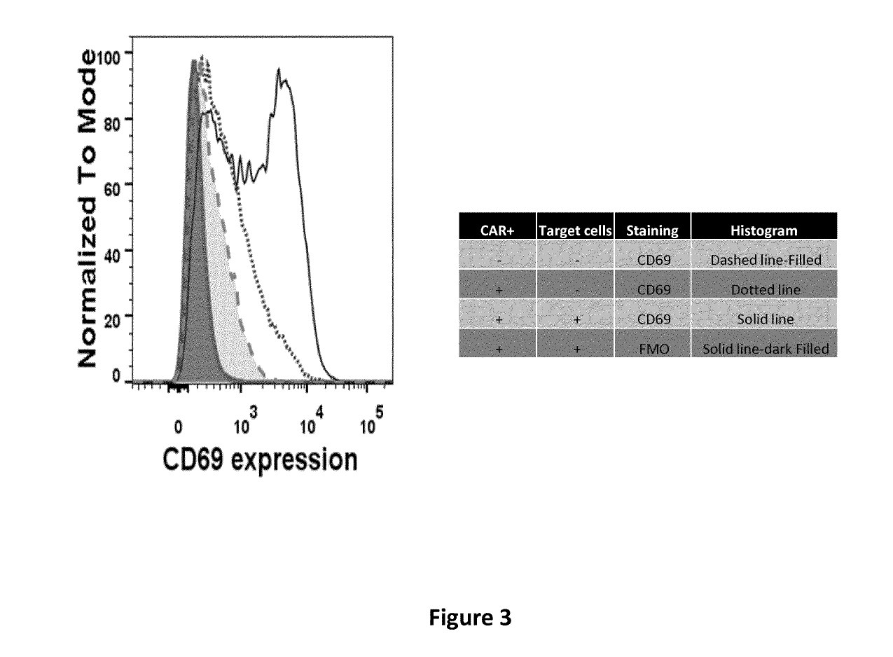

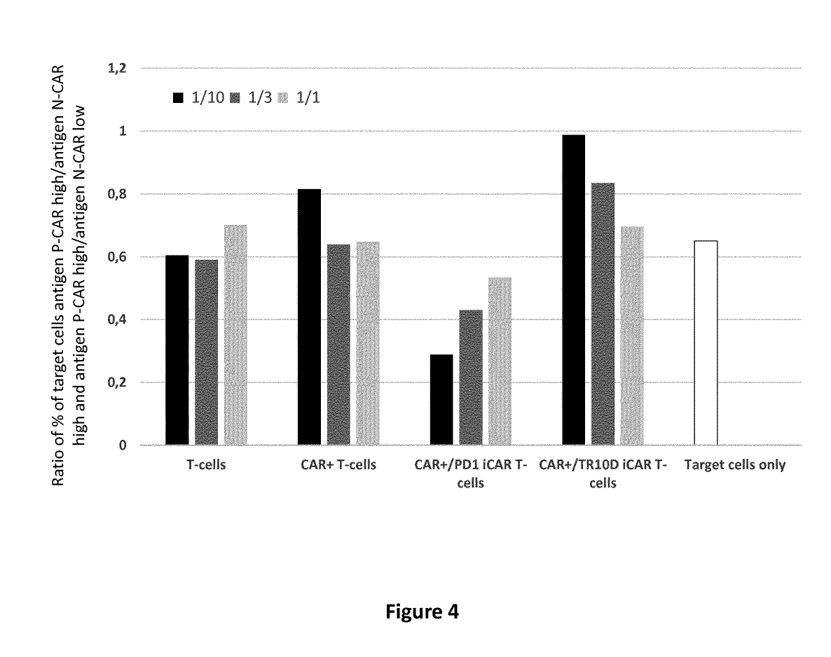

Example of a tetrameric P-CARs are illustrated in FIG. 3 of WO2013176915 and different versions of multichain P-CARs are represented in FIG. 4 of WO2013176915. Such P-CAR can be expressed in a T-cell obtained using the above disclosed method together with a N-CAR according to the present disclosure to obtain a T-cell according to the invention.

In some embodiment the invention relates to an immune cell comprising a N-CAR as defined herein and a P-CAR as defined in any of U.S. Pat. No. 7,446,190, WO2008/121420, U.S. Pat. No. 8,252,592, US20140024809, WO2012/079000, WO2014153270, WO2012/099973, WO2014/011988, WO2014/011987, WO2013/067492, WO2013/070468, WO2013/040557, WO2013/126712, WO2013/126729, WO 2013/126726, WO2013/126733, U.S. Pat. No. 8,399,645, US20130266551, US20140023674, WO2014039523, U.S. Pat. No. 7,514,537, U.S. Pat. No. 8,324,353, WO2010/025177, U.S. Pat. No. 7,446,179, WO2010/025177, WO2012/031744, WO2012/136231A1, WO2012/050374A2, WO2013074916, WO/2009/091826A3, WO2013/176915 or WO/2013/059593.

The transmembrane domain of the P-CAR responds to similar criteria that the one explained previously for the N-CAR. Idem for the extracellular ligand-binding domain of P-CAR, excepted the difference of specificity towards its antigen target as presented above.

A preferred TM is from CD8 alpha, more preferably of SEQ ID NO. 50

Example of a tetrameric P-CARs are illustrated in FIG. 3 of WO2013176915 and different versions of multichain P-CARs are represented in FIG. 4 of WO2013176915. Such P-CAR can be expressed in a T-cell obtained using the above disclosed method together with a N-CAR according to the present disclosure to obtain a T-cell according to the invention.

In some embodiment the invention relates to an immune cell comprising a N-CAR as defined herein and a P-CAR as defined in any of U.S. Pat. No. 7,446,190, WO2008/121420, U.S. Pat. No. 8,252,592, US20140024809, WO2012/079000, WO2014153270, WO2012/099973, WO2014/011988, WO2014/011987, WO2013/067492, WO2013/070468, WO2013/040557, WO2013/126712, WO2013/126729, WO 2013/126726, WO2013/126733, U.S. Pat. No. 8,399,645, US20130266551, US20140023674, WO2014039523, U.S. Pat. No. 7,514,537, U.S. Pat. No. 8,324,353, WO2010/025177, U.S. Pat. No. 7,446,179, WO2010/025177, WO2012/031744, WO2012/136231A1, WO2012/050374A2, WO2013074916, WO/2009/091826A3, WO2013/176915 or WO2013/059593.

The signaling domain of the p-CAR or “signaling protein” according to the invention is involved in the activation of at least one of the normal functions of the engineered immune cell. For example, the function of a T cell can be a cytolytic activity or helper activity including the secretion of cytokines. Thus, the term “signaling protein” refers to a protein which transduces the transmitter domain function signal and directs the cell to perform a specialized function. In a particular embodiment, said signaling domain can be a signaling protein. Transmission of the signals can result from: protein/protein interactions, protein/DNA interaction, protein/RNA interaction, protein/small molecule interaction, post translational protein modification, conformational change, subcellular relocalization.

The signaling protein can activate a gene in the nucleus. Examples of signaling protein can be members of NFAT transcription factor family which are inducible factor that could bind the intereukin-2 promoter in activated T cells. The regulation of NFAT proteins involves metabolites and proteins such as calcium, calcineurin and Homer scaffolding proteins. Said signaling protein can be an activated engineered form of NFAT avoiding regulation by calcineurin and Homer proteins. Said signaling protein can be a NF-κB engineered to avoid sequestration in the cytoplasm by I [symbol] b allowing activation of T cells. Said signaling protein can also be the expression of the three IKK subunits (IKKα, IKKβ, IKKγ). Reconstituted IKK complex activated NF- [symbol] B pathway, by triggering the ubiquitination of the IκB. Also the activation of the JNK signaling could be triggered through the direct expression of signaling protein AP-1 (transcription factor). Said signaling protein can be an engineered transcription activator like effector (TALE) binding domain that will specifically target and activate transcription of the same gene as for the NFAT and NF-kb.

According to the invention, said signaling protein can inhibit a signaling pathway through protein-protein interaction or can activate a gene in the nucleus to inhibit a signaling pathway. Said signaling protein can be vaccinia H1 related proteins (VHR) a member of the mitogen-activated protein kinase phosphatases (MKPs) family which dephosphorylates and inactivates an extracellular signal regulated kinases (ERK) signaling proteins.

According to the invention, a signal transducing domain for use in a P-CAR can be the cytoplasmic sequences of the T cell receptor and co-receptors that act in concert to initiate signal transduction following antigen receptor engagement, as well as any derivate or variant of these sequences and any synthetic sequence that has the same functional capability. Signal transduction domain may comprise two distinct classes of cytoplasmic signaling sequence, those that initiate antigen-dependent primary activation, and those that act in an antigen-independent manner to provide a secondary or co-stimulatory signal.

Primary cytoplasmic signaling sequence can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the invention can include as non limiting examples those derived from TCRzeta, FcRgamma, FcRbeta, FcRepsilon, CD3gamma, CD3delta, CD3epsilon, CD5, CD22, CD79a, CD79b and CD66d. In a preferred embodiment, the signaling transducing domain of a multi-chain CAR according to the invention can comprise a CD3zeta signaling domain, or the intracytoplasmic domain of the FcRI beta or gamma chains.

In particular embodiment the signal transduction domain of the P-CAR of the present invention comprises a co-stimulatory signal molecule. A co-stimulatory molecule is a cell surface molecule other than an antigen receptor or their ligands that is required for an efficient immune response. “Co-stimulatory ligand” refers to a molecule on an antigen presenting cell that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A “co-stimulatory molecule” refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor.

A co-stimulatory ligand according to the present invention can include but is not limited to CD7, B7-1 (CD80), B7-2 (CD86), PD-L1, PD-L2, 4-1BBL, OX40L, inducible costimulatory igand (ICOS-L), intercellular adhesion molecule (ICAM, CD30L, CD40, CD70, CD83, HLA-G, MICA, M 1CB, HVEM, lymphotoxin beta receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-IBB, OX40, CD30, CD40, PD-1, ICOS, lymphocyte function-associated antigen-1 (LFA-1), CD2, CD7, LTGHT, NKG2C, B7-H3, a ligand that specifically binds with CD83.

As preferred and exemplary P-CARs which can be used in combination with the N-CARs such as presented above, are those with an extracellular-binding domain recognizing the CD19, CD123, CD38, CS1, ROR1, CLL-1 or CD22 cell surface marker antigen.

Others P-CARs which can be used in combination with a N-CAR according to the present invention are contemplated within the present invention, such as anti-CD28 CAR, anti-CD30 CAR, anti-CD138 CAR, anti-CD171 CAR, anti-CD19 CAR, anti-CEA CAR (CEA being the carcinoembryonic antigen), anti-ERB B CAR (ligand of HER-2/neu), anti-FAP CAR (Fibroblast activation protein), anti-GD2 CAR, anti-GPC3 CAR (glypican-3 antigen), anti-Lewis-Y CAR (carbohydrate antigen), anti-NKG2D ligand CAR, anti-MSLN CAR (mesothelin antigen), anti-NY-ESO-1 CAR (cancer-testis antigen), anti-PSCA CAR (Prostate stem cell antigen), anti-GPC3 (glypican 3 antigen) CAR, anti-CD20 CAR, anti-HER1 CAR (EGFR/HER-1 oncoantigen) or anti-CD47 CAR. These P-CARs may have a single-chain or a multi-chain architecture.

CD123 P-CAR

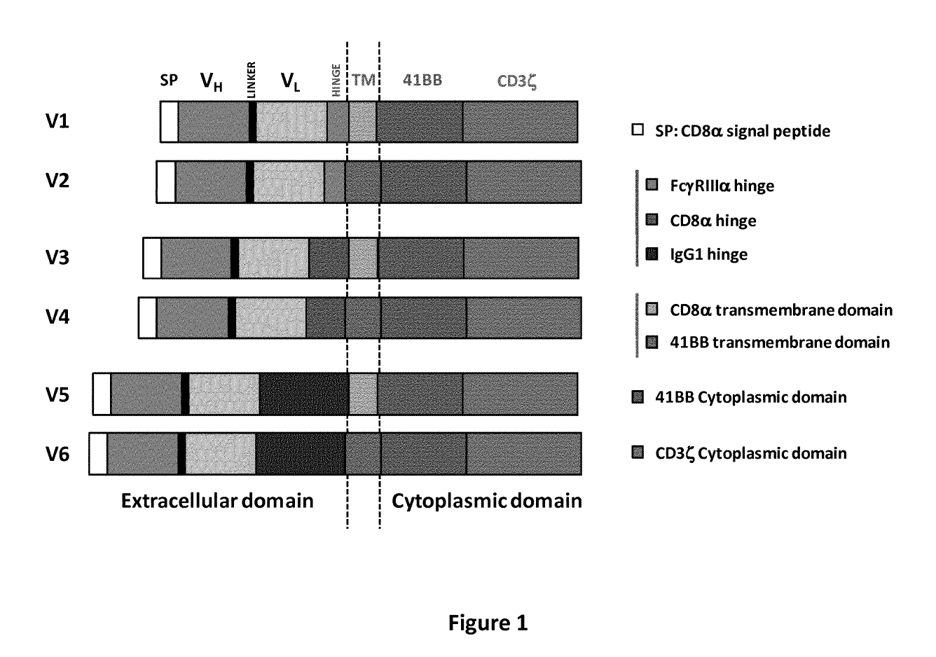

According to one embodiment, the P-CAR which is expressed in the engineered immune cell in combination with the N-CAR is a CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

As a preferred variant, VH and VL from a monoclonal anti-CD123 antibody which can be used are derived from KIon-43 (respectively SEQ ID NO:47-48).

As possible options, the following respective short, medium or long hinges from FcγRIIIα, CD8a, IgG1 (SEQ ID NO:49, SEQ ID NO:50, SEQ ID NO:51) can be used.

As preferred transmembrane domain, 4-1BB or CD8a (SEQ ID NO:53, SEQ ID NO:52) can be preferred, and more preferably CD8a.

In a preferred embodiment, the P-CAR which is expressed in the engineered immune cell in combination with the N-CAR is CD123 specific chimeric antigen receptor having one of the polypeptide structure selected from V1, V3 and V5, as illustrated in FIG. 1, said structure comprising an extra cellular ligand binding-domain comprising VH and VL from a monoclonal anti-CD123 antibody, a hinge, a transmembrane domain, a cytoplasmic domain including a CD3 zeta signaling domain and a co-stimulatory domain from 4-1BB, said 123 CAR having at least 80% sequence identity with either SEQ ID NO. 53, SEQ ID NO. 58 or SEQ ID NO. 60.

CS1 P-CAR

According to another embodiment, the P-CAR which is expressed in the engineered immune cell in combination with the N-CAR is a CS1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CS1 antibody, a hinge, a transmembrane domain and a cytoplasmic domain including a CD3 zeta signaling domain and a co-stimulatory domain from 4-1BB.

Concerning the VH chain and the VL from a monoclonal anti-CS1 antibody, they can derived from the murine scFv Luc63, Luc90, Luc34, LucX1 and LucX2 antibodies (SEQ ID NO:38 to 47 in WO 2015121454 A1), and optionally humanized from these.

CD38 P-CAR

According to another embodiment, the P-CAR which is expressed in the engineered immune cell in combination with the N-CAR is a CD38 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD38 antibody, a hinge, a transmembrane domain and a cytoplasmic domain including a CD3 zeta signaling domain and a co-stimulatory domain from 4-166.

Preferably, the anti-CD38 CAR as P-CAR comprises a polypeptide sequence displaying at least 90%, at least 95%, at least 98% or at least 99% identity with a sequence selected from the group consisting of SEQ ID NO. 64-66 (based on 25A10 mAb), SEQ ID NO. 67-69 (based on 28F5 mAb), SEQ ID NO. 70-72 (based 1665).

For these CD38 scCAR and CS1 scCAR, the choice of preferred hinge or transmembrane domains remains the same than for the CD123 CAR.

Although, cells expressing CD38, as well as many other tumor antigen markers CS1 could be regarded as attractive targets for CARs, the fact that such antigen markers are also expressed at the surface of most T-cells, has hampered significantly the selection of these markers to perform immunotherapy. Thus, according to a preferred embodiment, the anti-CD38 positive CAR or the anti-CS1 positive CAR is expressed in combination with a N-CAR in immune cells which are further engineered to inactivate such CD38 or CS1 expressed on the surface of said immune cell. This method is described in WO2015/121454. This gene inactivation may be performed by the use of specific endonuclease such as a TALE-nuclease.

CLL-1 P-CAR

According to another embodiment, the P-CAR according to the invention which is expressed in the engineered immune cell in combination with the N-CAR is a CLL-1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CLL-1 antibody, a hinge, a transmembrane domain and a cytoplasmic domain including a CD3 zeta signaling domain and a co-stimulatory domain from 4-1BB.

Said V_{L} and V_{H} from a monoclonal anti-CLL-1 antibody are preferably selected from the antibodies referred to in the literature as SCO02-357, SCO2-378 and SCO2-161 in WO2005/00894 (Applicant: Crucell Holland BV); M26, M31, G4, M22, M29, M2, M5, G12 in WO2013/169625 (Applicant: Cellerant Therapeutics); and 21.26, 1075.7 in WO2009/051974 (Applicant: Nuvelo Inc).

The choice of preferred hinge or transmembrane domains remains the same than for the previous scCARs.

CD22 P-CAR

According to another embodiment, the P-CAR which is expressed in the engineered immune cell in combination with the N-CAR is a CD22 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD22 antibody, a hinge, a transmembrane domain and a cytoplasmic domain including a CD3 zeta signaling domain and a co-stimulatory domain from 4-1BB.

P-CAR Level of Inactivation

In some embodiments, the immune cell of the invention is activated when the P-CAR antigen binding domain binds to its antigen. In some embodiments, such activation is reduced when the N-CAR antigen binding domain binds to its antigen.

In some embodiments such reduction of activation is increased, preferably by at least 5%, 10%, 15%, 20% or 30% in an immune cell comprising an N-CAR according to the invention as compared to the same immune cell comprising an N-CAR comprising the full intracellular domain of PD-1.

In some embodiments such reduction of activation is increased, preferably by at least 5%, 10%, 15%, 20% or 30% in an immune cell comprising an N-CAR according to the invention as compared to the same immune cell comprising an N-CAR comprising the full intracellular domain of CTLA-4.

In some embodiments, the activation due to P-CAR binding to its antigen is reduced by at least 5%, 10%, 15%, 20%, 25%, 30%, 35%, 40%, 45%, 50%, 55%, 60%, 65%, 70%, 75%, 80%, 85% when the N-CAR and P-CAR antigen binding domains, both, bind to their respective antigens as compared to when the P-CAR antigen binding domain, alone, binds to its antigen.

In some embodiments, the level of activation of the immune cell is measured by determining cytokine production.

In some embodiments, the level of activation of the immune cell is measured by monitoring IFNgamma production by ELISA and/or FACS and/or luminex assay.

In some embodiments, the level of activation of the immune cell is measured by monitoring TNFalpha production by ELISA and/or luminex assay.

In some embodiments, the level of activation of the immune cell is measured by monitoring degranulation, for example by measuring CD107a levels by FACS.

In some embodiments, the level of activation of the immune cell is measured by monitoring the ability of the immune cell to kill target cells.

In some embodiments, the negative signal of the N-CAR is short-termed and reversible to ensure that the immune cells comprising a P-CAR and an N-CAR according to the invention may be activated when it encounters only P-CAR antigen, despite prior inactivation in a off-tissue setting that has both P-CAR and N-CAR antigens.

mAb-Specific Epitope/Mimotope

According to another embodiment, the present invention relates to improved inhibitory chimeric antigen receptors (iCAR), wherein the extracellular binding domain (scFv) has been modified by insertion of at least one mAb-specific epitope.

Such insertion is designed to allow both sorting and/or depletion of the immune cells endowed with said N-CARs.

According to another embodiment, the immune cell has been further engineered to express a P-CAR in which of such at least one mAb-specific epitope is inserted.

Preferably, two mAb-specific epitopes are inserted.

Such epitope(s) is(are) inserted anywhere in the extracellular part of N-CAR or P-CAR, either in the N terminal part, between the VH and VL chains of the scFvs or between the hinge or linker and the scFvs. Preferably, when more than one mAb-specific epitope are used, they are not in tandem (side by side).

In a preferred embodiment, the epitope introduced within the chimeric scFv is the CD20 antigen and the infused mAb which is being used to target it for sorting and/or depletion purpose(s) is rixutimab. Such epitope target sequence has over 80% identity, preferably over 90%, and more preferably over 95% identity, more preferably 100% identity with the CD20 antigen of SEQ ID NO. 82. Such preferred suicide gene system employs a recombinant antigenic polypeptide comprising antigenic motif recognized by the anti-CD20 mAb Rituximab, especially QBen10, such as in the so-called RQR8 polypeptide described in WO2013153391. Rituximab, an authorized antibody drug, can then be used for cell depletion when needed.

According to another embodiment, the epitope is a mimotope. As a macromolecule, often a peptide, which mimics the structure of an epitope, the mimotope has the advantage to be smaller than conventional epitope, and therefore may be beneficial for a non-conformational sequence and easier to reproduce in a long polypeptide such a CAR. Mimotopes are known for several pharmaceutically-approved mAb such as two 10 amino acid peptides for cetuximab (Riemer et al., 2005), or a 24 aa for palivizumab (Arbiza et al, 1992). As these mimotopes can be identified by phage display, it is possible to try several of them in order to obtain a sequence which does not perturb the scFv for the same mAb. Furthermore, their use can enhance a complement-dependent cytotoxicity (CDC).

As exemples, mimotopes of CD20 is SEQ ID NO:73 (CPYSNPSLC), mimotopes corresponding to the use of cetuximab of SEQ ID NO: 74 (CQFDLSTRRLKC) SEQ ID NO: 75 (CQYNLSSRALKC) SEQ ID NO: 76 (CVWQRWQKSYVC), SEQ ID NO: 77 (CMWDRFSRWYKC); mimotopes corresponding to the use of palivizumab of SEQ ID NO: 78 (NSELLSLINDMPITNDQKKLMSNN) or mimotopes corresponding to the use of nivolumab of SEQ ID NO: 79 (SFVLNWYRMSPSNQTDKLAAFPEDR), SEQ ID NO: 80 (SGTYLCGAISLAPKAQIKE).

The present invention relates also to the immune cells expressing said N-CARs, to the methods of in vivo depleting and/or in vitro sorting said CAR-expressing immune cells, and is drawn to their therapeutic use.

Isolated Immune Cell

Cell according to the present invention refers to a cell of hematopoietic origin functionally involved in the initiation and/or execution of innate and/or adaptative immune response. Cell according to the present invention is preferably a T-cell obtained from a donor. Said T cell according to the present invention can be derived from a stem cell. The stem cells can be adult stem cells, embryonic stem cells, more particularly non-human stem cells, cord blood stem cells, progenitor cells, bone marrow stem cells, totipotent stem cells or hematopoietic stem cells. In a preferred embodiment, cells are human cells, in particular human stem cells.

Representative human stem cells are CD34+ cells. Said isolated cell can also be a dendritic cell, killer dendritic cell, a mast cell, a NK-cell, a B-cell or a T-cell selected from the group consisting of inflammatory T-lymphocytes, cytotoxic T-lymphocytes, regulatory T-lymphocytes or helper T-lymphocytes. In another embodiment, said cell can be derived from the group consisting of CD4+ T-lymphocytes and CD8+ T-lymphocytes. Prior to expansion and genetic modification of the cells of the invention, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T-cell lines available and known to those skilled in the art, may be used. In another embodiment, said cell is preferably derived from a healthy donor. In another embodiment, said cell is part of a mixed population of cells which present different phenotypic characteristics.

Preferably, isolation and preparation of stem cells does not require the destruction of at least one human embryo. The immune cells can originate from the patient, in view of operating autologous treatments, or from donors in view of producing allogeneic cells, which can be used in allogeneic treatments.

The present invention relates also to an isolated immune cell comprising a P-CAR and an N-CAR such as presented above.

Said P-CAR may be a single chain CAR or a multi-chain P-CAR as defined in WO2014/039523.

According to an embodiment, the isolated immune cell includes at least a N-CAR which comprises at least:
an extracellular binding domain, which is able to bind to an “off-target” antigen on a healthy cell and;
an inhibitory transmembrane polypeptide having a sequence with more than 80%, preferably 90% and more preferably 95%, and even more preferably 100% identity with a sequence from SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, which is able to bind to an “off-target” antigen on a healthy cell;
an inhibitory transmembrane polypeptide having a sequence selected from the group consisting of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a P-CAR which comprises in the engineered immune cell in combination with the N-CAR; said P-CAR comprising:

one transmembrane polypeptide comprising at least one extracellular ligand-binding domain able to bind to CD123 antigen, and one signal-transducing domain, optionally with a co-stimulatory domain.

According to an embodiment, the isolated immune cell includes at least a N-CAR which comprises at least:
an extracellular binding domain, comprising a polypeptide sequence sharing more than 80%, preferably 90% and more preferably 95% and even more preferably 100% identity with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);
an inhibitory transmembrane polypeptide having a sequence with more than 80%, preferably 90% and more preferably 95% identity and even more preferably 100% with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);
an inhibitory transmembrane polypeptide having a sequence of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:
an extracellular binding domain, which is able to bind to an “off-target” antigen on a healthy cell, and
an inhibitory transmembrane polypeptide having a polypeptide sequence selected from the group consisting of SEQ ID NO: 33 (human TR10D), SEQ ID NO: 34 (human TR10A) and SEQ ID NO: 35 (human TR10B);

and a P-CAR which comprises in the engineered immune cell in combination with the N-CAR; said P-CAR comprising:
one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, which is able to bind to an “off-target” antigen on a healthy cell and;
an inhibitory transmembrane polypeptide having a sequence of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:

an extracellular binding domain, comprising a polypeptide sequence sharing more than 80%, preferably 90% and more preferably 95% identity and even more preferably 100% with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);

and;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);
and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);
an inhibitory transmembrane polypeptide having a polypeptide sequence of more than 80%, preferably 90% and more preferably 95% identity with SEQ ID NO: 33 (human TR10D), SEQ ID NO: 34 (human TR10A) and SEQ ID NO: 35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);
an inhibitory transmembrane polypeptide having a polypeptide sequence of SEQ ID NO: 33 (human TR10D), SEQ ID NO: 34 (human TR10A) and SEQ ID NO: 35 (human TR10B).

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a more preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);

and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);
and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

Preferably, the above anti-CD123 CARs (P-CARs) having one of the polypeptide structure selected from V1, V3 and V5, as illustrated in FIG. 1, said structure comprising an extra cellular ligand binding-domain comprising VH and VL from a monoclonal anti-CD123 antibody, a hinge, a transmembrane domain, a cytoplasmic domain including a CD3 zeta signaling domain and a co-stimulatory domain from 4-1BB, said 123 CAR having at least 80% sequence identity with either SEQ ID NO. 56, SEQ ID NO. 58 or SEQ ID NO. 60.

According to an embodiment, the isolated immune cell includes at least a N-CAR which comprises at least:
an extracellular binding domain, comprising a polypeptide sequence sharing more than 80%, preferably 90% and more preferably 95% identity and even more preferably 100% identity with SEQ ID NO: 86 (CD56 antigen), SEQ ID NO: 87 (CD205 antigen), SEQ ID NO: 88 (CD83 antigen), SEQ ID NO: 89 (CD206 antigen), SEQ ID NO: 90 (CD200 antigen), or SEQ ID NO: 91 (CD36 antigen);

and;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);
and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CD38 specific chimeric antigen receptor (CAR) (as P-CAR) which comprises in the engineered immune cell in combination with the N-CAR; said CD38 specific chimeric antigen receptor (CAR) containing at least one transmembrane polypeptide which includes at least one extra-binding domain recognizing specifically the CD38 antigen, and an intracellular signaling domain, optionally with co-stimulatory domain.

According to an embodiment, the isolated immune cell includes at least a N-CAR which comprises at least:

an extracellular binding domain, which is able to bind to an “off-target” antigen on a healthy cell;

and;
an inhibitory transmembrane polypeptide of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CD38 specific chimeric antigen receptor (CAR) (as P-CAR) which comprises in the engineered immune cell in combination with the N-CAR; said CD38 specific chimeric antigen receptor (CAR) containing at least one transmembrane polypeptide which includes at least one extra-binding domain recognizing specifically the CD38 antigen, and an intracellular signaling domain, optionally with co-stimulatory domain.

According to a preferred embodiment, the isolated immune cell includes at least a N-CAR which comprises at least:

an extracellular binding domain, comprising a polypeptide sequence sharing more than 80%, preferably 90% and more preferably 95% identity and even more preferably 100% identity with SEQ ID NO: 86 (CD56 antigen), SEQ ID NO: 87 (CD205 antigen), SEQ ID NO: 88 (CD83 antigen), SEQ ID NO: 89 (CD206 antigen), SEQ ID NO: 90 (CD200 antigen), or SEQ ID NO: 91 (CD36 antigen); and;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CD38 specific chimeric antigen receptor (CAR) (as P-CAR) which comprises in the engineered immune cell in combination with the N-CAR; said CD38 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD38 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% identity with SEQ ID NO: 86 (CD56 antigen), SEQ ID NO: 87 (CD205 antigen), SEQ ID NO: 88 (CD83 antigen), SEQ ID NO: 89 (CD206 antigen), SEQ ID NO: 90 (CD200 antigen), or SEQ ID NO: 91 (CD36 antigen);

and;
an inhibitory transmembrane polypeptide having a sequence of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CD38 specific chimeric antigen receptor (CAR) (as P-CAR) which comprises in the engineered immune cell in combination with the N-CAR; said CD38 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD38 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% identity and even more preferably 100% identity with SEQ ID NO: 86 (CD56 antigen), SEQ ID NO: 87 (CD205 antigen), SEQ ID NO: 88 (CD83 antigen), SEQ ID NO: 89 (CD206 antigen), SEQ ID NO: 90 (CD200 antigen), or SEQ ID NO: 91 (CD36 antigen);

and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);

and a CD38 specific chimeric antigen receptor (CAR) (as P-CAR) which comprises in the engineered immune cell in combination with the N-CAR; said CD38 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD38 antibody, a hinge, a transmembrane domain and a cytoplasmic domain including a CD3 zeta signaling domain and a co-stimulatory domain from 4-1BB.

Preferably, the anti-CD38 CAR as P-CAR comprises a polypeptide sequence displaying at least 90%, at least 95%, at least 98% or at least 99% identity to one selected from SEQ ID NO. 64-66 (based on 25A10 mAb), SEQ ID NO. 67-69 (based on 28F5 mAb) or SEQ ID NO. 70-72 (based on 16B5 mAb).

According to a more preferred embodiment, the anti-CD38 specific chimeric antigen receptor (anti-CD38 CAR) of the invention comprises a polypeptide sequence displaying at least 90%, at least 95%, at least 98% or at least 99% identity to one selected from SEQ ID NO. 64-66 (based on 25A10 mAb) or SEQ ID NO. 67-69 (based on 28F5 mAb).

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);
and a CS1 specific chimeric antigen receptor (CAR) (as P-CAR) which comprises in the engineered immune cell in combination with the N-CAR; said CS1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CS1 specific chimeric antigen receptor (CAR) (as P-CAR) which comprises in the engineered immune cell in combination with the N-CAR; said CS1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to an embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen);
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CS1 specific chimeric antigen receptor (CAR) (as P-CAR) which comprises in the engineered immune cell in combination with the N-CAR; said CS1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CS1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen);
an inhibitory transmembrane polypeptide of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CS1 specific chimeric antigen receptor (CAR) (as P-CAR) which comprises in the engineered immune cell in combination with the N-CAR; said CS1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CS1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);
and a CS1 specific chimeric antigen receptor (CAR) (as P-CAR) which comprises in the engineered immune cell in combination with the N-CAR; said CS1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to an embodiment, the isolated immune cell includes at least a N-CAR which comprises at least:

an extracellular binding domain, which is able to bind to an “off-target” antigen on a healthy cell;

and;
an inhibitory transmembrane polypeptide having a polypeptide sequence of SEQ ID NO: 33 (human TR10D), SEQ ID NO: 34 (human TR10A) and SEQ ID NO: 35 (human TR10B);
and a CS1 specific chimeric antigen receptor (CAR) (as P-CAR) which comprises in the engineered immune cell in combination with the N-CAR; said CS1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to a preferred embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen); and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);

and a CS1 specific chimeric antigen receptor (CAR) (as P-CAR) which comprises in the engineered immune cell in combination with the N-CAR; said CS1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CS1 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen); and;
an inhibitory transmembrane polypeptide having a polypeptide sequence of SEQ ID NO: 33 (human TR10D), SEQ ID NO: 34 (human TR10A) and SEQ ID NO: 35 (human TR10B);

and a CS1 specific chimeric antigen receptor (CAR) (as P-CAR) which comprises in the engineered immune cell in combination with the N-CAR; said CS1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CS1 antibody, a hinge, a transmembrane domain and a cytoplasmic domain including a CD3 zeta signaling domain and a co-stimulatory domain from 4-1BB.

Preferably, the above CS1 specific chimeric antigen receptor (CAR) (as P-CAR) comprises an extra cellular ligand binding-domain in which VH and VL chains derive from a monoclonal anti-CS1 antibody, such as the murine scFv Luc63, Luc90, Luc34, LucX1 and LucX2 antibodies (such as described in WO2015121454A1 SEQ ID NO. 38 to 47) optionally humanized.

According to an embodiment, the isolated immune cell includes at least a N-CAR which comprises at least:
an extracellular binding domain, which is able to bind to an “off-target” antigen on a healthy cell;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);
and a ROR1 specific chimeric antigen receptor (CAR) (as P-CAR) which comprises in the engineered immune cell in combination with the N-CAR; said ROR1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to an embodiment, the isolated immune cell includes at least a N-CAR which comprises at least:
an extracellular binding domain, which is able to bind to an “off-target” antigen on a healthy cell;
an inhibitory transmembrane polypeptide having a sequence of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);
and a ROR1 specific chimeric antigen receptor (CAR) (as P-CAR) which comprises in the engineered immune cell in combination with the N-CAR; said ROR1 specific chimeric antigen receptor (CAR) containing at least one transmembrane polypeptide which includes at least one extra-binding domain recognizing specifically the CS1 antigen, and an intracellular signaling domain, optionally with co-stimulatory domain.

According to an embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen);
an inhibitory transmembrane polypeptide having of more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a ROR1 specific chimeric antigen receptor (CAR) (as P-CAR) which comprises in the engineered immune cell in combination with the N-CAR; said ROR1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-ROR1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen);
an inhibitory transmembrane polypeptide having a of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a ROR1 specific chimeric antigen receptor (CAR) (as P-CAR) which comprises in the engineered immune cell in combination with the N-CAR; said ROR1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-ROR1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);
and a ROR1 specific chimeric antigen receptor (CAR) (as P-CAR) which comprises in the engineered immune cell in combination with the N-CAR; said ROR1 specific chimeric antigen receptor (CAR) containing at least one transmembrane polypeptide which includes at least one extra-binding domain recognizing specifically the ROR1 antigen, and an intracellular signaling domain, optionally with co-stimulatory domain.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a polypeptide sequence of SEQ ID NO: 33 (human TR10D), SEQ ID NO: 34 (human TR10A) and SEQ ID NO: 35 (human TR10B);
and a ROR1 specific chimeric antigen receptor (CAR) (as P-CAR) which comprises in the engineered immune cell in combination with the N-CAR; said ROR1 specific chimeric antigen receptor (CAR) containing at least one transmembrane polypeptide which includes at least one extra-binding domain recognizing specifically the ROR1 antigen, and an intracellular signaling domain, optionally with co-stimulatory domain.

According to a preferred embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen); and;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);

and a ROR1 specific chimeric antigen receptor (CAR) (as P-CAR) which comprises in the engineered immune cell in combination with the N-CAR; said ROR1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-ROR1 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% and even more preferably 100% identity identity with SEQ ID NO: 92 (troponin C), SEQ ID NO: 93 (beta-1 integrin), SEQ ID NO: 94 (CCKBR antigen), SEQ ID NO: 95 (GALR1 antigen) or SEQ ID NO: 96 (CUBN antigen); and;
an inhibitory transmembrane polypeptide having a polypeptide sequence of SEQ ID NO: 33 (human TR10D), SEQ ID NO: 34 (human TR10A) and SEQ ID NO: 35 (human TR10B);

and a ROR1 specific chimeric antigen receptor (CAR) (as P-CAR) which comprises in the engineered immune cell in combination with the N-CAR; said ROR1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-ROR1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which binds to an “off-site” cell surface antigen expressed in healthy cells or in immune cells;
an inhibitory transmembrane polypeptide having a sequence with more than 80%, preferably 90% and more preferably 95% identity and even more preferably 100% identity with SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CLL-1 specific chimeric antigen receptor (CAR) (as P-CAR) which comprises in the engineered immune cell in combination with the N-CAR; said CLL-1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CLL-1 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which binds to an “off-site” cell surface antigen expressed in healthy cells or in immune cells;
an inhibitory transmembrane polypeptide having a sequence of SEQ ID NO: 1 (human KI2L2), SEQ ID NO: 2 (human KI2L1), SEQ ID NO:3 (human FCG2B), SEQ ID NO:4 (human KI2L3), SEQ ID NO: 5 (human KI3L2), SEQ ID NO:6 (human KI2L4), SEQ ID NO:7 (human KI3L1), SEQ ID NO:8 (human KI2LA), SEQ ID NO:9 (human MILR1), SEQ ID NO:10 (human LIRB4), SEQ ID NO:11 (human LIRB3), SEQ ID NO:12 (human KI3L3), SEQ ID NO:15 (human LIRB5), SEQ ID NO:16 (human LIRB2), SEQ ID NO:18 (human FCRL4), SEQ ID NO:23 (human FCRL5), SEQ ID NO:24 (human FCRL2), SEQ ID NO: 25 (human FCRL1), SEQ ID NO:27 (human FCRL3), SEQ ID NO:28 (human MPZL1), SEQ ID NO:29 (human PILRA) or SEQ ID NO:30 (human PVR);

and a CLL-1 specific chimeric antigen receptor (CAR) (as P-CAR) which comprises in the engineered immune cell in combination with the N-CAR; said CLL-1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CLL-1 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which binds to an “off-site” cell surface antigen expressed in healthy cells or in immune cells;
an inhibitory transmembrane polypeptide having a polypeptide sequence of more than 80%, preferably 90% and more preferably 95% identity and even more preferably 100% identity with SEQ ID NO: 33 (human TR10D), SEQ ID NO: 34 (human TR10A) and SEQ ID NO: 35 (human TR10B);

and a CLL-1 specific chimeric antigen receptor (CAR) (as P-CAR) which comprises in the engineered immune cell in combination with the N-CAR; said CLL-1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CLL-1 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which binds to an “off-site” cell surface antigen expressed in healthy cells or in immune cells;
an inhibitory transmembrane polypeptide having a polypeptide sequence of SEQ ID NO: 33 (human TR10D), SEQ ID NO: 34 (human TR10A) and SEQ ID NO: 35 (human TR10B);

and a CLL-1 specific chimeric antigen receptor (CAR) (as P-CAR) which comprises in the engineered immune cell in combination with the N-CAR; said CLL-1 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CLL-1 antibody, a hinge, a transmembrane domain and a cytoplasmic domain including a CD3 zeta signaling domain and a co-stimulatory domain from 4-1BB.

As examples, said V_{L} and V_{H} are preferably selected from the antibodies referred to in the literature as SCO02-357, SCO2-378 and SCO2-161 in WO2005/00894 (Applicant: Crucell Holland BV); M26, M31, G4, M22, M29, M2, M5, G12 in WO2013/169625 (Applicant: Cellerant Therapeutics); and 21.26, 1075.7 in WO2009/051974 (Applicant: Nuvelo Inc).

According to a preferred embodiment, the isolated immune cell includes at least a N-CAR which comprises at least:
an extracellular binding domain, which is able to bind to an “off-target” antigen on a healthy or immune cell and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, which is able to bind to an “off-target” antigen on a healthy or immune cell and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to an embodiment, the isolated immune cell includes at least a N-CAR which comprises at least:
an extracellular binding domain, comprising a polypeptide sequence sharing more than 80%, preferably 90% and more preferably 95% identity with SEQ ID NO: 81 (CD4 antigen), SEQ ID NO: 82 (CD20 antigen), SEQ ID NO: 83 (CD22 antigen), SEQ ID NO: 84 (CD25 antigen) or SEQ ID NO: 85 (MUC1 antigen);
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD4 antigen), SEQ ID NO: XX (CD20 antigen), SEQ ID NO: XX (CD22 antigen), SEQ ID NO: XX (CD25 antigen) or SEQ ID NO: XX (MUC1 antigen);
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:
an extracellular binding domain, which is able to bind to an “off-target” antigen on a healthy or immune cell and;
an inhibitory transmembrane polypeptide having a polypeptide sequence of SEQ ID NO: 33 (human TR10D), SEQ ID NO: 34 (human TR10A) and SEQ ID NO: 35 (human TR10B).

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to another preferred embodiment, the isolated immune cell includes at least a N-CAR which comprises at least:
an extracellular binding domain, which is able to bind to an “off-target” antigen on a healthy or immune cell and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD4 antigen), SEQ ID NO: XX (CD20 antigen), SEQ ID NO: XX (CD22 antigen), SEQ ID NO: XX (CD25 antigen) or SEQ ID NO: XX (MUC1 antigen)

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);
and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD4 antigen), SEQ ID NO: XX (CD20 antigen), SEQ ID NO: XX (CD22 antigen), SEQ ID NO: XX (CD25 antigen) or SEQ ID NO: XX (MUC1 antigen);
an inhibitory transmembrane polypeptide having a polypeptide sequence of more than 80%, preferably 90% and more preferably 95% identity with SEQ ID NO: 33 (human TR10D), SEQ ID NO: 34 (human TR10A) and SEQ ID NO: 35 (human TR10B);

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a preferred embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD4 antigen), SEQ ID NO: XX (CD20 antigen), SEQ ID NO: XX (CD22 antigen), SEQ ID NO: XX (CD25 antigen) or SEQ ID NO: XX (MUC1 antigen);
an inhibitory transmembrane polypeptide having a polypeptide sequence of SEQ ID NO: 33 (human TR10D), SEQ ID NO: 34 (human TR10A) and SEQ ID NO: 35 (human TR10B).

and a P-CAR which comprises in the engineered immune cell in combination with the N-CAR; said P-CAR comprising one transmembrane polypeptide comprising at least one extracellular ligand-binding domain able to bind to CD123 antigen, and one signal-transducing domain, optionally with a co-stimulatory domain.

According to a more preferred embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD4 antigen), SEQ ID NO: XX (CD20 antigen), SEQ ID NO: XX (CD22 antigen), SEQ ID NO: XX (CD25 antigen) or SEQ ID NO: XX (MUC1 antigen)

and;
an inhibitory transmembrane polypeptide having a polypeptide sequence of more than 80%, preferably 90% and more preferably 95% identity with SEQ ID NO: 33 (human TR10D), SEQ ID NO: 34 (human TR10A) and SEQ ID NO: 35 (human TR10B);

and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

Preferably, the above anti-CD123 CARs (P-CARs) having one of the polypeptide structure selected from V1, V3 and V5, as illustrated in FIG. 1, said structure comprising an extra cellular ligand binding-domain comprising VH and VL from a monoclonal anti-CD123 antibody, a hinge, a transmembrane domain, a cytoplasmic domain including a CD3 zeta signaling domain and a co-stimulatory domain from 4-1BB, said 123 CAR having at least 80% sequence identity with either SEQ ID NO. 42, SEQ ID NO. 44 or SEQ ID NO. 46.

According to an embodiment, the isolated immune cell includes at least a N-CAR which comprises at least:
an extracellular binding domain, comprising a polypeptide sequence sharing more than 80%, preferably 90% and more preferably 95% identity with SEQ ID NO: XX (CD56 antigen), SEQ ID NO: XX (CD205 antigen), SEQ ID NO: XX (CD83 antigen), SEQ ID NO: XX (CD206 antigen), SEQ ID NO: XX (CD200 antigen), or SEQ ID NO: 8 (CD36 antigen);

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);
and a CD123 specific chimeric antigen receptor (CAR) (as P-CAR) which comprises in the engineered immune cell in combination with the N-CAR; said CD123 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD123 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD38 specific chimeric antigen receptor (CAR) (as P-CAR) which comprises in the engineered immune cell in combination with the N-CAR; said CD38 specific chimeric antigen receptor (CAR) containing at least one transmembrane polypeptide which includes at least one extra-binding domain recognizing specifically the CD38 antigen, and an intracellular signaling domain, optionally with co-stimulatory domain.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD38 specific chimeric antigen receptor (CAR) (as P-CAR) which comprises in the engineered immune cell in combination with the N-CAR; said CD38 specific chimeric antigen receptor (CAR) containing at least one transmembrane polypeptide which includes at least one extra-binding domain recognizing specifically the CD38 antigen, and an intracellular signaling domain, optionally with co-stimulatory domain.

According to a preferred embodiment, the isolated immune cell includes at least a N-CAR which comprises at least:
an extracellular binding domain, which is able to bind to an “off-target” antigen on a healthy or immune cell;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD19 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD19 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which is able to bind to an “off-target” antigen on a healthy cell;

and;
an inhibitory transmembrane polypeptide having a polypeptide sequence of more than 80%, preferably 90% and more preferably 95% identity with SEQ ID NO: 33 (human TR10D), SEQ ID NO: 34 (human TR10A) and SEQ ID NO: 35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD38 antibody, a hinge, a transmembrane domain and a cytoplasmic domain including a CD3 zeta signaling domain and a co-stimulatory domain from 4-1BB.

According to an embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD19 specific chimeric antigen receptor (CAR) (as P-CAR) which comprises in the engineered immune cell in combination with the N-CAR; said CD19 specific chimeric antigen receptor (CAR) containing at least one transmembrane polypeptide which includes at least one extra-binding domain recognizing specifically the CD19 antigen, and an intracellular signaling domain, optionally with co-stimulatory domain.

According to a preferred embodiment, the isolated immune cell includes at least a N-CAR which comprises at least:

an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD19 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which is able to bind to an “off-target” antigen on a healthy cell;

and;
an inhibitory transmembrane polypeptide having a sequence consisting essentially of amino acids No 201-375 from SEQ ID NO:8 (human KI2LA), amino acids No 206-348 from SEQ ID NO:2 (human KIR2DL1), amino acids No 206-348 from SEQ ID NO:1 (human KIR2DL2), amino acids No 206-341 from SEQ ID NO:4 (human KIR2DL3), amino acids No 206-348 from SEQ ID NO:2 (human KIR2DL1), amino acids No 203-377 from SEQ ID NO:6 (human KIR2DL4), amino acids No 301-444 from SEQ ID NO:7 (human KIR3DL1), amino acids No 301-455 from SEQ ID NO:5 (human KIR3DL2), amino acids No 204-310 from SEQ ID NO:24 (human FRGR2B), amino acids No 214-343 from SEQ ID NO:9 (human MILR1), amino acids No 216-448 from SEQ ID NO:10 (human LIRB4), amino acids No 420-631 from SEQ ID NO:11 (human LIRB3), amino acids No 296-410 from SEQ ID NO:12 (human KI3L3), amino acids No 419-590 from SEQ ID NO:15 (human LIRB5), amino acids No 420-598 from SEQ ID NO:16 (human LIRB2), amino acids No 375-515 from SEQ ID NO:18 (human FCRL4), amino acids No 753-977 from SEQ ID NO:23 (human FCRL5), amino acids No 388-508 from SEQ ID NO:24 (human FCRL2), amino acids No 292-429 from SEQ ID NO: 25 (human FCRL1), amino acids No 564-734 from SEQ ID NO:27 (human FCRL3), amino acids No 147-269 from SEQ ID NO:28 (human MPZL1), amino acids No 151-303 from SEQ ID NO:29 (human PILRA), amino acids No 329-417 from SEQ ID NO:30 (human PVR), amino acids No 229-325 from SEQ ID NO:36 (human CD200 receptor1), amino acids No 181-386 from SEQ ID NO:33 (human TR10D), amino acids No 230-468 from SEQ ID NO:34 (human TR10A) or amino acids No 179-440 from SEQ ID NO:35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD19 antibody, a hinge, a transmembrane domain and a cytoplasmic domain including a CD3 zeta signaling domain and a co-stimulatory domain from 4-1BB.

According to a preferred embodiment, the isolated immune cell includes at least a N-CAR which comprises at least:
an extracellular binding domain, which is able to bind to an “off-target” antigen on a healthy cell;

and;
an inhibitory transmembrane polypeptide having a polypeptide sequence of more than 80%, preferably 90% and more preferably 95% identity with SEQ ID NO: 33 (human TR10D), SEQ ID NO: 34 (human TR10A) and SEQ ID NO: 35 (human TR10B);

and a CD19 specific chimeric antigen receptor (CAR) (as P-CAR) which comprises in the engineered immune cell in combination with the N-CAR; said CD19 specific chimeric antigen receptor (CAR) having one of the polypeptide structure selected from V1 to V6, preferably V1, V3 and V5 as illustrated in FIG. 1, said structure comprising an extra cellular ligand binding-domain comprising VH and VL from a monoclonal anti-CD19 antibody, a hinge, a transmembrane domain and a cytoplasmic domain including a CD3 zeta signaling domain and a co-stimulatory domain from 4-1BB.

Said immune cell engineered to express both the N-CAR and the P-CAR such as presented previously is intended to be used as a medicament.

Preferably, such engineered immune cell is intended to be used for the treatment of cancer.

More preferably, such engineered immune cell is intended to be used for the treatment of refractory relapsed cancer.

By “relapsed cancer”, it is referred to a cancer that returns after a period of improvement. This applies whether the cancer was treated or untreated.

By “refractory cancer”, it is referred to a cancer that proves resistant, or does not respond to, treatment, regardless whether the cancer is resistant to treatment immediately, or it develops a resistance during treatment.

Methods of Engineering Immune Cells

The inventors developed methods of engineering such immune cells based on the rational combination of regulatory modules in artificial circuits for performing tasks based on “NOT gates”. The term “gate” is used to refer to a device or molecular mechanism that produces a particular (predetermined) output in response to two or more input signals. According to the present invention, the logical NOT gate refers to the immune cell inhibition, in particular T cell cytotoxicity against a target cell through inhibition of specific proteins (signaling proteins) resulting from the concomitant binding to 2 different antigens.

In one embodiment, the method of engineering an immune can comprise the steps of:

(a) Providing an immune cell;

(b) Expressing the N-CAR and the P-CAR at the surface of said cell.

In another embodiment, the method of engineering an immune cell can comprise the steps of:

a. Introducing into said cell at least one polynucleotide encoding the N-CAR and at least one polynucleotide encoding the CAR;

b. Expressing said polynucleotides into said cell.

P-CARs and immune cells comprising them have been extensively disclosed and can be prepared by the skilled person according to known methods. For example, a methodology to prepare P-CAR and cells comprising such P-CARs is disclosed in U.S. Pat. No. 7,446,190, WO2008/121420, U.S. Pat. No. 8,252,592, US20140024809, WO2012/079000, WO2014153270, WO2012/099973, WO2014/011988, WO2014/011987, WO2013/067492, WO2013/070468, WO2013/040557, WO2013/126712, WO2013/126729, WO 2013/126726, WO2013/126733, U.S. Pat. No. 8,399,645, US20130266551, US20140023674, WO2014039523, U.S. Pat. No. 7,514,537, U.S. Pat. No. 8,324,353, WO2010/025177, U.S. Pat. No. 7,446,179, WO2010/025177, WO2012/031744, WO2012/136231A1, WO2012/050374A2, WO2013074916, WO2009/091826A3, WO2013/176915 or WO/2013/059593 which are all incorporated herein in their entirety by reference. Immune cells comprising a P-CAR and a N-CAR can be prepared by the skilled person according to the methodologies disclosed in the above mentioned references. In a preferred embodiment, immune cells comprising a P-CAR and a N-CAR can be prepared by the skilled person according to the methodology disclosed in WO2013/176915.

In one embodiment, the method of engineering T-cells of invention can comprise:
(a) Providing a T-cell, preferably from a cell culture or from a blood sample;
(b) Transforming said T cell with a nucleic acid encoding a rare-cutting endonuclease able to

Selectively inactivate by DNA cleavage, preferably by double-strand break respectively at least one gene encoding a component of the T-cell receptor (TCR);
(d) Expressing said rare-cutting endonucleases into said T-cells;
(e) Sorting the transformed T-cells, which do not express TCR on their cell surface;

In some embodiments, the method of engineering T-cells of invention can comprise:
(a) Providing a T-cell, preferably from a cell culture or from a blood sample;
(b) Selecting a gene in said T-cell expressing a target for an immunosuppressive agent;
(c) Transforming said T cell with nucleic acid encoding a rare-cutting endonuclease able to selectively inactivate by DNA cleavage, preferably by double-strand break respectively:
said gene encoding a target for said immunosuppressive agent, and
at least one gene encoding a component of the T-cell receptor (TCR);
(d) Expressing said rare-cutting endonucleases into said T-cells;
(e) Sorting the transformed T-cells, which do not express TCR on their cell surface;
(f) Expanding said cells, optionally in presence of said immunosuppressive agent.

Such inactivation of TCR gene may be performed such as described in the Example 1 of the application WO2014/184143.

In some embodiment, the method to engineer A cell of the invention further comprises one or more additional genomic modification step. By additional genomic modification step, can be intended the introduction into cells to engineer of one or more protein of interest. Said protein of interest can be a P-CAR and/or an N-CAR.

By “Immunosuppressive agents” or “immunosuppressive agents”, it is meant drugs that inhibit or prevent activity of the immune system.

In some embodiments, the method of engineering T-cells of invention can comprise:
(a) modifying T-cells by inactivating at least:
   a first gene expressing a target for an immunosuppressive agent, and
   a second gene encoding a component of the T-cell receptor (TCR)
(b) expanding said cells, optionally in presence of said immunosuppressive agent.

An immunosuppressive agent is an agent that suppresses immune function by one of several mechanisms of action. In other words, an immunosuppressive agent is a role played by a compound which is exhibited by a capability to diminish the extent and/or voracity of an immune response. As non-limiting example, an immunosuppressive agent can be a calcineurin inhibitor, a target of rapamycin, an interleukin-2 u-chain blocker, an inhibitor of inosine monophosphate dehydrogenase, an inhibitor of dihydrofolic acid reductase, a corticosteroid or an immunosuppressive antimetabolite.

In a particular embodiment, the genetic modification step of the method relies on the inactivation of one gene selected from the group consisting of CD52, GR, TCR alpha and TCR beta. In another embodiment, the genetic modification step of the method relies on the inactivation of two genes selected from the group consisting of dCK, CD52 and GR, CD52 and TCR alpha, CDR52 and TCR beta, GR and TCR alpha, GR and TCR beta, TCR alpha and TCR beta. In another embodiment, the genetic modification step of the method relies on the inactivation of more than two genes. The genetic modification is preferably operated ex-vivo.

Inactivation of CD52, CTLA-4 and/or PD-1 genes, for instance by TALE-nuclease, may be performed such as described respectively in Examples 2, 3 and 4 in the application WO2014/184744.

The rare-cutting endonucleases used for inactivating the genes in T-cells are preferably Transcription Activator like Effector (TALE), but may be also a Cas9 coupled to a RNA guide as respectively described in WO 2013/176915 and WO 2014/191128.

Compositions/Formulations

Another aspect of the present invention relates to compositions or formulations containing genetically engineered immune cells which express at least one N-CAR and at least one P-CAR such as described above and at least one pharmaceutically acceptable carrier or vehicle.

Compositions of the invention comprising genetically modified immune cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminium monostearate and gelatin. According to the present invention, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the genetically modified immunoresponsive cells as described in the present invention. This will present no problem to those skilled in chemical and pharmaceutical principles, or problems can be readily avoided by reference to standard texts or by simple experiments (not involving undue experimentation), from this disclosure and the documents cited herein.

The skilled artisan can readily determine the amount of cells and optional additives, vehicles, and/or carrier in compositions and to be administered in methods of the invention. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. Of course, for any composition to be administered to an animal or human, and for any particular method of administration, it is preferred to determine therefore: toxicity, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Delivery Methods

The different methods described above involve expressing N-CAR and P-CAR at the surface of a cell. As non-limiting example, said N-CAR and P-CAR can be expressed by introducing the latter into a cell. CARs can be introduced as transgene encoded by one plasmidic vector. Said plasmid vector can also contain a selection marker which provides for identification and/or selection of cells which received said vector.

Polypeptides may be synthesized in situ in the cell as a result of the introduction of polynucleotides encoding said polypeptides into the cell. Alternatively, said polypeptides could be produced outside the cell and then introduced thereto. Methods for introducing a polynucleotide construct into cells are known in the art and including as non-limiting examples stable transformation methods wherein the polynucleotide construct is integrated into the genome of the cell, transient transformation methods wherein the polynucleotide construct is not integrated into the genome of the cell and virus mediated methods. Said polynucleotides may be introduced into a cell by for example, recombinant viral vectors (e.g. retroviruses, adenoviruses), liposome and the like. For example, transient transformation methods include for example microinjection, electroporation or particle bombardment. Said polynucleotides may be included in vectors, more particularly plasmids or virus, in view of being expressed in cells.

Polynucleotides and Vectors

In one embodiment, said isolated cell according to the present invention comprises a polynucleotide encoding the “NOT gate” receptor (N-CAR & P-CAR).

The present invention also relates to polynucleotides, vectors encoding the above described N-CAR and P-CAR according to the invention.

The polynucleotide may consist in an expression cassette or expression vector (e.g. a plasmid for introduction into a bacterial host cell, or a viral vector such as a baculovirus vector for transfection of an insect host cell, or a plasmid or viral vector such as a lentivirus for transfection of a mammalian host cell).

In a particular embodiment, the different nucleic acid sequences can be included in one polynucleotide or vector which comprises a nucleic acid sequence encoding ribosomal skip sequence such as a sequence encoding a 2A peptide. 2A peptides, which were identified in the Aphthovirus subgroup of picornaviruses, causes a ribosomal “skip” from one codon to the next without the formation of a peptide bond between the two amino acids encoded by the codons (see (Doronina, Wu et al. 2008, Mol Cell Biol 28(13):4227-39). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct, transmembrane polypeptide into the secretory pathway of a host cell, a secretory signal sequence (also known as a leader sequence, prepro sequence or pre sequence) is provided in polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830).

Those skilled in the art will recognize that, in view of the degeneracy of the genetic code, considerable sequence variation is possible among these polynucleotide molecules. Preferably, the nucleic acid sequences of the present invention are codon-optimized for expression in mammalian cells, preferably for expression in human cells. Codon-optimization refers to the exchange in a sequence of interest of codons that are generally rare in highly expressed genes of a given species by codons that are generally frequent in highly expressed genes of such species, such codons encoding the amino acids as the codons that are being exchanged.

Therapeutic Applications

In another embodiment, isolated cell expressing both at least one N-CAR and at least one P-CAR obtained by the different methods or cell line derived from said isolated cell as previously described can be used as a medicament.

In another embodiment, said medicament can be used for treating cancer in a patient in need thereof.

In another embodiment, said isolated cell according to the invention or cell line derived from said isolated cell can be used in the manufacture of a medicament for treatment of a cancer in a patient in need thereof.

In another embodiment, said engineered cell expressing both at least one N-CAR and at least one P-CAR is intended for its use in therapy, wherein the condition is a haematological cancer condition. In particular, such haematological cancer condition is leukemia.

More specifically, such engineered cell is intended for its use in therapy, wherein said leukemia is selected from the group consisting of acute myelogenous leukemia (AML), chronic myelogenous leukemia, melodysplastic syndrome, acute lymphoid leukemia, chronic lymphoid leukemia (CLL), and myelodysplastic syndrome.

In a preferred embodiment, said engineered cell is intended to be used in therapy, wherein the condition is a pre-malignant or malignant cancer condition characterized by CD123-expressing cells. In particular, such condition is characterized by an overabundance of CD123-expressing cells.

In a particular embodiment, such engineered cell is intended for its use in therapy, wherein the leukemia is acute myelogenous leukemia (AML). Therefore, this is particularly adapted for treating the pre-malignant or malignant cancer AML condition characterized especially by CD123-expressing cells or by CLL-1 expressing cells.

In a preferred embodiment, said engineered cell is intended to be used in therapy, wherein the condition is a pre-malignant or malignant cancer condition such as multiple myeloma (MM) characterized especially by CD38-expressing cells.

In another particular embodiment, such engineered cell is intended for its use in therapy, wherein the leukemia is chronic lymphocytic leukemia (CLL). Therefore, this is particularly adapted for treating the pre-malignant or malignant cancer CLL condition characterized especially by CS1-expressing cells.

In another particular embodiment, said engineered cell is intended to be used in therapy, wherein the condition is a pre-malignant or malignant cancer CLL condition characterized by ROR1-expressing cells.

In another embodiment, said engineered cell for use in therapy, wherein said malignant lymphoproliferative disorder is lymphoma. More specifically, such engineered cell may be used to treat lymphoma is selected from the group consisting of multiple myeloma, non-Hodgkin's lymphoma, Burkitt's lymphoma, and follicular lymphoma (small cell and large cell).

In another particular embodiment, said engineered cell is intended to be used in therapy, wherein the condition is a solid tumor such as breast, colon, lung, or kidney tumor characterized especially by ROR1-expressing cells.

In a preferred embodiment, said engineered cell is intended to be used in therapy, wherein the condition is a pre-malignant or malignant cancer condition characterized by CD22-expressing cells.

In another aspect, the present invention relies on methods for treating patients in need thereof, said method comprising at least one of the following steps:

(a) providing an immune-cell obtainable by any one of the methods previously described;

(b) Administrating said transformed immune cells to said patient,

On one embodiment, said T cells of the invention can undergo robust in vivo T cell expansion and can persist for an extended amount of time.

Said treatment can be ameliorating, curative or prophylactic. It may be either part of an autologous immunotherapy or part of an allogenic immunotherapy treatment. By autologous, it is meant that cells, cell line or population of cells used for treating patients are originating from said patient or from a Human Leucocyte Antigen (HLA) compatible donor. By allogeneic is meant that the cells or population of cells used for treating patients are not originating from said patient but from a donor.

Cells that can be used with the disclosed methods such as TALE nuclease.

Said treatment can be used to treat patients diagnosed with cancer, viral infection, autoimmune disorders or Graft versus Host Disease (GvHD). Cancers that may be treated include tumors that are not vascularized, or not yet substantially vascularized, as well as vascularized tumors. The cancers may comprise non solid tumors (such as hematological tumors, for example, leukemias and lymphomas) or may comprise solid tumors. Types of cancers to be treated with the N-CAR and P-CAR of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

It can be a treatment in combination with one or more therapies against cancer selected from the group of antibodies therapy, chemotherapy, cytokines therapy, dendritic cell therapy, gene therapy, hormone therapy, laser light therapy and radiation therapy.

The administration of the cells or population of cells according to the present invention may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10¹⁰ cells per kg body weight, preferably 10⁵ to 10⁶ cells/kg body weight including all integer values of cell numbers within those ranges. The cells or population of cells can be administrated in one or more doses. In another embodiment, said effective amount of cells are administrated as a single dose. In another embodiment, said effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, said effective amount of cells or composition comprising those cells are administrated parenterally. Said administration can be an intravenous administration. Said administration can be directly done by injection within a tumor.

In certain embodiments of the present invention, cells are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efaliztimab treatment for psoriasis patients or other treatments for PML patients. In further embodiments, the T cells of the invention may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and irradiation. These drugs inhibit either the calcium dependent phosphatase calcineurin (cyclosporine and FK506) or inhibit the p7056 kinase that is important for growth factor induced signaling (rapamycin) (Henderson, Naya et al. 1991, Immunology 73(3):316-21; Liu, Albers et al. 1992, 31(16):3896-901; Bierer, Hollander et al. 1993, Curr Opin Immunol 5(5):763-73). In a further embodiment, the cell compositions of the present invention are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH.

In another embodiment, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
By chimeric antigen receptor (CAR) is intended molecules that combine a binding domain against a component present on the target cell, for example an antibody-based specificity for a desired antigen (e.g., tumor antigen) with a T cell receptor-activating intracellular domain to generate a chimeric protein that exhibits a specific anti-target cellular immune activity. Generally, CAR consists of an extracellular single chain antibody (scFv) fused to the intracellular signaling domain of the T cell antigen receptor complex zeta chain (scFv:ζ) and have the ability, when expressed in T cells, to redirect antigen recognition based on the monoclonal antibody's specificity.
By “delivery vector” or “delivery vectors” is intended any delivery vector which can be used in the present invention to put into cell contact (i.e “contacting”) or deliver inside cells or subcellular compartments (i.e “introducing”) agents/chemicals and molecules (proteins or nucleic acids) needed in the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles, emulsions or other appropriate transfer vectors. These delivery vectors allow delivery of molecules, chemicals, macromolecules (genes, proteins), or other vectors such as plasmids, peptides developed by Diatos. In these cases, delivery vectors are molecule carriers. By “delivery vector” or “delivery vectors” is also intended delivery methods to perform transfection.
The terms “vector” or “vectors” refer to a nucleic acid molecule capable of transporting another nucleic acid to which it has been linked. A “vector” in the present invention includes, but is not limited to, a viral vector, a plasmid, a RNA vector or a linear or circular DNA or RNA molecule which may consists of a chromosomal, non chromosomal, semi-synthetic or synthetic nucleic acids. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e. g. adenoassociated viruses), coronavirus, negative strand RNA viruses such as orthomyxovirus (e. g., influenza virus), rhabdovirus (e. g., rabies and vesicular stomatitis virus), paramyxovirus (e. g. measles and Sendai), positive strand RNA viruses such as picornavirus and alphavirus, and double-stranded DNA viruses including adenovirus, herpesvirus (e. g., Herpes Simplex virus types 1 and 2, Epstein-Barr virus, cytomegalovirus), and poxvirus (e. g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996).
By “lentiviral vector” is meant HIV-Based lentiviral vectors that are very promising for gene delivery because of their relatively large packaging capacity, reduced immunogenicity and their ability to stably transduce with high efficiency a large range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration in the DNA of infected cells. By “integrative lentiviral vectors (or LV)”, is meant such vectors as nonlimiting example, that are able to integrate the genome of a target cell. At the opposite by “non-integrative lentiviral vectors (or NILV)” is meant efficient gene delivery vectors that do not integrate the genome of a target cell through the action of the virus integrase.
Delivery vectors and vectors can be associated or combined with any cellular permeabilization techniques such as sonoporation or electroporation or derivatives of these techniques.
by “mutation” is intended the substitution, deletion, insertion of up to one, two, three, four, five, six, seven, eight, nine, ten, eleven, twelve, thirteen, fourteen, fifteen, twenty, twenty five, thirty, fourty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “functional variant” is intended a catalytically active mutant of a protein or a protein domain; such mutant may have the same activity compared to its parent protein or protein domain or additional properties, or higher or lower activity.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
The term “subject” or “patient” as used herein includes all members of the animal kingdom including non-human primates and humans.
By “Transcription Activator like Effector (TALE)” it is meant a binding domain protein wherein sequence specificity is driven by a series of 33-35 amino acids repeats originating from Xanthomonas or Ralstonia bacterial proteins. These repeats differ essentially by two amino acids positions that specify an interaction with a base pair (Boch, Scholze et al. 2009, Science 326(5959):1509-12; Moscou and Bogdanove 2009, Science 326(5959):1501). Each base pair in the DNA target is contacted by a single repeat, with the specificity resulting from the two variant amino acids of the repeat (the so-called repeat variable dipeptide, RVD). TALE binding domains may further comprise an N-terminal translocation domain responsible for the requirement of a first thymine base (To) of the targeted sequence and a C-terminal domain that containing a nuclear localization signals (NLS). A TALE nucleic acid binding domain generally corresponds to an engineered core TALE scaffold comprising a plurality of TALE repeat sequences, each repeat comprising a RVD specific to each nucleotides base of a TALE recognition site. In the present invention, each TALE repeat sequence of said core scaffold is made of 30 to 42 amino acids, more preferably 33 or 34 wherein two critical amino acids (the so-called repeat variable dipeptide, RVD) located at positions 12 and 13 mediates the recognition of one nucleotide of said TALE binding site sequence; equivalent two critical amino acids can be located at positions other than 12 and 13 specially in TALE repeat sequence taller than 33 or 34 amino acids long. Preferably, RVDs associated with recognition of the different nucleotides are HD for recognizing C, NG for recognizing T, NI for recognizing A, NN for recognizing G or A. In another embodiment, critical amino acids 12 and 13 can be mutated towards other amino acid residues in order to modulate their specificity towards nucleotides A, T, C and G and in particular to enhance this specificity. A TALE nucleic acid binding domain usually comprises between 8 and 30 TALE repeat sequences. More preferably, said core scaffold of the present invention comprises between 8 and 20 TALE repeat sequences; again more preferably 15 TALE repeat sequences. It can also comprise an additional single truncated TALE repeat sequence made of 20 amino acids located at the C-terminus of said set of TALE repeat sequences, i.e. an additional C-terminal half-TALE repeat sequence.

By “primary cell” or “primary cells” are intended cells taken directly from living tissue (i.e. biopsy material) and established for growth in vitro, that have undergone very few population doublings and are therefore more representative of the main functional components and characteristics of tissues from which they are derived from, in comparison to continuous tumorigenic or artificially immortalized cell lines.

General Methods

Primary T-Cell Cultures

T cells were purified from Buffy coat samples using Ficoll gradient density medium. The PBMC layer was recovered and T cells were purified using a commercially available T-cell enrichment kit. Purified T cells were activated in X-Vivo™-15 medium (Lonza) supplemented with 20 ng/mL Human IL-2, 5% Human, and Dynabeads Human T activator CD3/CD28 at a bead:cell ratio 1:1 (Life Technologies).

CAR mRNA Transfection

Transfections were done at Day 4 or Day 11 after T-cell purification and activation. 5 millions of cells were transfected with 15 μg of mRNA encoding the different CAR constructs. CAR mRNAs were produced using T7 mRNA polymerase transfections done using Cytopulse technology, by applying two 0.1 mS pulses at 3000V/cm followed by four 0.2 mS pulses at 325V/cm in 0.4 cm gap cuvettes in a final volume of 200 μl of “Cytoporation buffer T” (BTX Harvard Apparatus). Cells were immediately diluted in X-Vivo™-15 media and incubated at 37° C. with 5% CO₂. IL-2 was added 2 h after electroporation at 20 ng/mL.

Degranulation Assay (CD107a Mobilization)

T-cells were incubated in 96-well plates (40,000 cells/well), together with an equal amount of cells expressing various levels of the P antigen (eg expression CD123 or CD20) and undetectable level of N antigen or together with an equal amount of cells expressing various levels of the P antigen (eg expression CD123 or CD20) and detectable level of N antigen as determined by flow cytometry analysis using appropriate control(s). Co-cultures were maintained in a final volume of 100 μl of X-Vivo™-15 medium (Lonza) for 6 hours at 37° C. with 5% CO₂. CD107a staining was done during cell stimulation, by the addition of a fluorescent anti-CD107a antibody at the beginning of the co-culture, together with 1 μg/ml of anti-CD49d, 1 μg/ml of anti-CD28, and 1× Monensin solution. After the 6 h incubation period, cells were stained with a fixable viability dye and fluorochrome-conjugated anti-CD8 and analyzed by flow cytometry. The degranulation activity was determined as the % of CD8+/CD107a+ cells, and by determining the mean fluorescence intensity signal (MFI) for CD107a staining among CD8+ cells. Degranulation assays were carried out 24 h after mRNA transfection.

IFN Gamma Release Assay

T-cells were incubated in 96-well plates (40,000 cells/well), together with cell lines expressing various levels of the P CAR and/or the N-CAR expressed protein. Co-cultures were maintained in a final volume of 100 μl of X-Vivo™-15 medium (Lonza) for 24 hours at 37° C. with 5% CO₂. After this incubation period the plates were centrifuged at 1500 rpm for 5 minutes and the supernatants were recovered in a new plate. IFN gamma detection in the cell culture supernatants was done by ELISA assay. The IFN gamma release assays were carried by starting the cell co-cultures 24 h after mRNA transfection.

Cytotoxicity Assay

T-cells were incubated in 96-well plates (100,000 cells/well), together with 10,000 target cells (expressing CD123) and 10,000 control (CD123neg) cells in the same well. Target and control cells were labelled with fluorescent intracellular dyes (CFSE or Cell Trace Violet) before co-culturing them with CAR+ T-cells. The co-cultures were incubated for 4 hours at 37° C. with 5% CO₂. After this incubation period, cells were labelled with a fixable viability dye and analyzed by flow cytometry. Viability of each cellular population (target cells or P CAR/NCAR neg control cells) was determined and the % of specific cell lysis was calculated. Cytotoxicity assays were carried out 48 h after mRNA transfection.

T-Cell Transduction

Transduction of T-cells with recombinant lentiviral vectors expression the CAR was carried out three days after T-cell purification/activation. CAR detection at the surface of T-cells was done using a recombinant protein consisting on the fusion of the extracellular domain of the human CAR expressed protein, together with a murine IgG1 Fc fragment. Binding of this protein to the CAR molecule was detected with a fluorochrome-conjugated secondary antibody targeting the mouse Fc portion of the protein, and analyzed by flow cytometry.

### EXAMPLES

### Example 1. Design of Inhibitory Gate Receptors

Sequences to construct N-CARs are obtained from the Uniprot database and were restricted to human proteins, excluding in addition type II membrane proteins (N-terminus on the cytoplasmic side of the membrane).

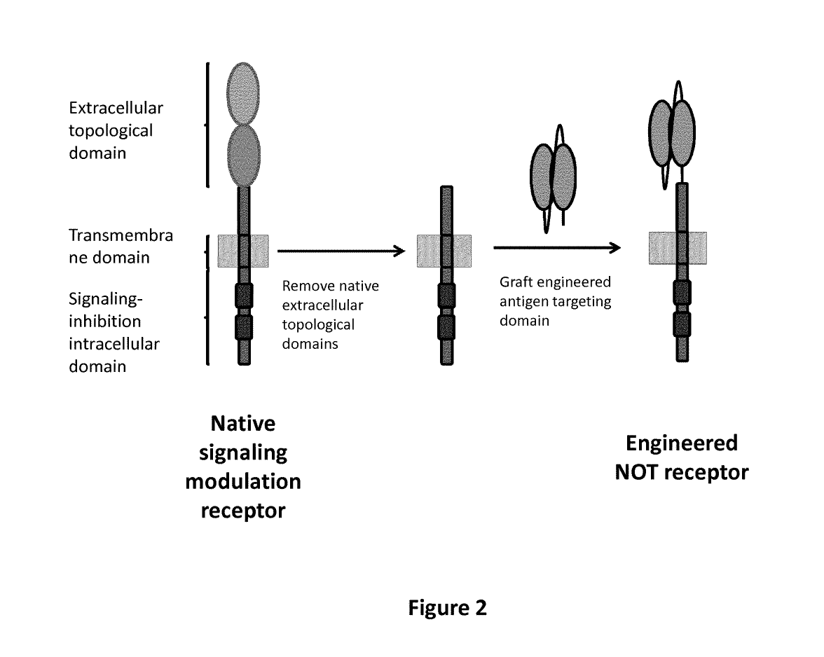

N-CAR are designed (such as schematized in FIG. 2) to be composed of an antigen targeting domain (anti-CD20 VH &VL of SEQ ID NO. 45-46, anti-BCMA VH & VL of SEQ ID NO. 41-42 and anti-PSMA VH & VL chain of SEQ ID NO. 43-44) fused via a short classical-GS-linker of SEQ ID NO. 39 or SEQ ID NO. 40 to the membrane receptor of interest of SEQ ID NO. 1-36 that included the whole cytoplasmic domain, the transmembrane domain and the amino acid sequence up to the first annotated extracellular topological domain. In case where the extracellular topological domains are not clearly annotated, the fusion point was determined based on other similar receptors. If the resulting extracellular domain was short, an additional portion of the first annotated extracellular topological domain was added.

N-CAR are cloned in a mammalian expression plasmid upstream a 2A cis-acting hydrolase element of SEQ ID NO. 97 followed by a reporter marker (e.g. fluorescent proteins) of SEQ ID NO. 98-99. Standard molecular biology technics such as PCR, enzymatic restriction digestion and ligation are applied to create all construction, leading to SEQ ID NO. 100-212.

The production of lentiviral particles to vectorize CARs and N-CAR is performed using commercially available Lentiviral Packaging Mix (Invitrogen) following the manufacturer protocols or, alternatively, the lentiviral particles are obtained directly from commercial manufacturers (Vectalys).

### Example 2. Characterization of N-CARS in Immortalized Human T-Cells

The P-CAR (expressing CD20 antigen; transduced by SEQ ID NO. 213) model cell line is generated by lentiviral transduction of an immortalized human T-cell line (Jurkat). The transduced cells are purified for positive surface CAR+ expression using bulk FACS sorting or magnetic separation. The whole bulk CAR+ population is then assessed for positive CAR+ driven activation (degranulation/cytotoxicity), proliferation, and cytokine release. The results are presented in FIG. 3. High, medium and low CAR+ expressing sub-population or clonal cells are identified and isolated.

The appropriate P-CAR (or CAR+) Jurkat cell line or population is then transfected with individual or combination of DNA plasmid encoding N-CARs such as presented in Example 1. The level of activation (degranulation/cytokine secretion) is assessed by FACS in P-CAR/N-CAR positive Jurkat cells using a model cell line expressing both the P-CAR and N-CAR target antigens, a model cell line expressing only the P-CAR antigen and a model cell line expressing only the N-CAR antigen.

The cytotoxicity of P-CAR/N-CAR positive cells, which are tested versus P-CAR positive cells in presence or absence of target cells, is presented in the following Table 3. The results are expressed as a ratio of CD69 fluorescence between the T cells expressing P-CAR/N-CAR or P-CAR in the presence of target cells and in the absence of target cells.

TABLE 3
Test of cytotoxicity of T cells encoding a P-CAR combined to diverse
N-CARs versus T cells P-CAR only when they are tested with or
without target cells (based on ratio mean CD69 fluorescence
intensity +/− target cells)

| Name of | | | |
|---|---|---|---|
| plasmid | | | |
| encoding | N-CAR | | |
| inhibitory | (inhibitory | | P-CAR & N- |
| sequence | sequence) | P-CAR | CAR |
| pCLS27705 | iFCRL2 | 1.51 | 1.13 |
| pCLS27706 | iFCRL3 | 1.58 | 1.14 |
| pCLS27707 | iFCRL4 | 1.62 | 1.15 |
| pCLS27698 | iCD200R | 1.68 | 1.14 |
| pCLS27708 | iFCRL5 | 1.82 | 1.19 |
| pCLS27699 | iTR10A | 1.76 | 1.01 |
| pCLS27709 | iLIRB2 | 1.68 | 1.17 |
| pCLS27700 | iTR10B | 1.62 | 0.97 |
| pCLS27711 | iLIRB4 | 1.67 | 1.07 |
| pCLS27701 | iTR10D | 1.67 | 1.22 |
| pCLS27712 | iLIRB5 | 1.70 | 1.14 |
| pCLS27713 | iMILR1 | 1.58 | 1.02 |
| pCLS27703 | iFCG2B | 1.66 | 1.14 |
| pCLS27714 | iMPZL1 | 1.65 | 1.14 |
| pCLS27704 | iFCRL1 | 1.72 | 1.10 |
| pCLS27715 | iPILRA | 1.62 | 1.05 |
| pCLS27716 | iPVR | 1.54 | 1.11 |
| pCLS27726 | iKI2L2 | 1.40 | 1.19 |
| pCLS27727 | iKI2L3 | 1.49 | 1.22 |
| pCLS27728 | iKI2L4 | 1.65 | 1.19 |
| pCLS27729 | iKI2LA | 1.64 | 1.21 |
| pCLS27730 | iKI3L1 | 1.70 | 1.13 |
| pCLS27731 | iKI3L2 | 1.62 | 1.25 |
| pCLS27732 | iKI3L3 | 1.64 | 1.15 |
| pCLS27733 | iPECA1 | 1.52 | 1.19 |
| pCLS27725 | iKI2L1 | 1.62 | 1.31 |

The results shown in the Table 3 that all the N-CARs present a significant inhibitory effect on the P-CAR (encoding CD20 scFvs). This is reflected by a marked reduction of the ratio of mean CD69 fluorescence intensity when the T cells endowing both N-CAR and P-CAR in presence of target cell, when compared to T cells endowing P-CAR only.

This is particularly the case for the N-CARs encoding the TRAIL inhibitory receptors TR10A, TR10B and TR10D.

### Example 3. Characterization of N-CARS in Primary T-Cells

N-CAR constructs allowing attenuation of the P-CAR signal (degranulation/cytokine secretion) identified according to Example 2 are subcloned in a lentiviral production plasmid using standard molecular biology, leading to SEQ ID. 214 to 217.

Primary T-cells are transduced sequentially using N-CAR lentiviral particles SEQ ID. 215 and SEQ ID NO. 217 and P-CAR of SEQ ID. 218. The N-CAR transduced T-cells are purified for positive surface expression using FACS sorting. The P-CAR positive population is then transduced with N-CAR lentiviral particles. Comparative effects of P-CAR/N-CAR and N-CAR engineered primary T-cells is assessed using a engineered target cell line (HEK293) that contained two major populations expressing the target antigens for the P-CAR and N-CAR (antigen P-CAR high/antigen N-CAR high and antigen P-CAR high/antigen N-CAR low). The target cell population is then incubated for 6 hours with the different engineered primary T-cells and the relative proportion of the two target populations (live cells expressing CD19 and PMSA antigens) is recorded.

The data clearly indicated that the antigen P-CAR-high/antigen N-CAR high target cell population is protected compared to the antigen CAR-high/antigen N-CAR low target cell in the presence of the TR10D engineered N-CAR. Indeed, an increase of the ratio of the percentage of live cells between the two target cell populations (at the three ratio of target/effectors are used: 1/1, 1/3 and 1/10, FIG. 4) is measured.

The data show a dose dependent effect in the reduction induced by N-CAR.

Surprisingly, when testing a N-CAR comprising the inhibitory molecule PD-1 in the same conditions, an increase in the ratio of % of target cells antigen P-CAR-high/antigen N-CAR high and antigen P-CAR-high/antigen N-CAR-low is measured.

1. An inhibitory chimeric antigen receptor (N-CAR) comprising:
an extracellular domain comprising an antigen binding domain;
a transmembrane domain; and
an intracellular domain;
wherein said N-CAR comprises a polypeptide sequence involved in inducing an inhibitory transduction signal,
said polypeptide sequence comprises at least one sequence from a Tumor-necrosis-factor related apoptosis inducing ligand (TRAIL) receptor or at least one sequence from a CD200 receptor 1.

2. The N-CAR according to claim 1, comprising a polypeptide sequence from a Tumor-necrosis-factor related apoptosis inducing ligand (TRAIL) receptor.

3. The N-CAR according to claim 1, comprising at least one polypeptide sequence from a polypeptide sequence selected from SEQ ID NO: 33 (human TR10D), SEQ ID NO: 34 (human TR10A) or SEQ ID NO: 35 (human TR10B) or fragments thereof.

4. The N-CAR according to claim 1, wherein said polypeptide sequence has more than 80% sequence identity with a sequence from SEQ ID NO: 33, SEQ ID NO: 34 or SEQ ID NO: 35 or a fragments thereof.

5. The N-CAR according to claim 1, comprising at least one of the following polypeptide sequences: amino acids 181-386 from SEQ ID NO: 33 (human TR10D), amino acids 230-468 from SEQ ID NO: 34 (human TR10A) or of amino acids 179-440 from SEQ ID NO: 35 (human TR10B), or a fragments thereof.

6. The N-CAR according to claim 1, wherein said antigen binding domain binds to a cell surface antigen N, N being not expressed on a cancerous cell and N being expressed on a non-cancerous cell or a healthy cell, said non-cancerous cell or a healthy cell also expressing a P antigen, said P antigen being also expressed or over expressed on a cancerous cell.

7. The N-CAR according to claim 1, wherein said antigen binding domain binds to at least one cell surface antigen N selected from CD56, CD205, CD83, CD206, CD200, CD36, troponin C, beta-1 integrin, CCKBR, GALR1 CUBN, CD4, CD20, CD22, CD25, MUC1, CD19, or PSMA.

8. A vector encoding a N-CAR according to claim 1.

9. An immune cell comprising a P-CAR comprising:
an extracellular domain comprising an antigen binding domain;
a transmembrane domain; and
an intracellular domain;
and a N-CAR according to claim 1.

10. The immune cell according to claim 9, wherein at least one gene encoding a TCR alpha or a TCR beta subunit is inactivated.

11. The immune cell according to claim 9 wherein at least one gene encoding a TCR and a gene encoding a deoxycytidine kinase (dck) are inactivated.

12. The immune cell according to claim 11, wherein the TCR and dck genes are inactivated by deletion using an endonuclease.

13. A method of treating or preventing a haematological cancer condition, comprising administering to a subject in need thereof a therapeutically effective amount of the immune cell according to claim 12.

14. The method according to claim 13, wherein said haematological cancer is leukemia or myeloma.

15. A method of engineering an immune cell according to claim 9 comprising:
(a) Providing an immune cell comprising optionally deleting a candidate gene, said candidate gene being TCRA or dCK;
(b) Expressing a N-CAR and a P-CAR at the cell surface; and
(c) optionally deleting a candidate gene, said candidate gene selected from TCRA, PD1, CTLA4 or dCK.

16. The method of claim 15, wherein said immune cells are from a donor.

17. The immune cell according to claim 9, wherein the immune cell is primary immune T-cell.

18. The immune cell according to claim 10, wherein the TCR alpha or the TCR beta subunit gene is inactivated by deletion using a specific endonuclease.

19. The method according to claim 13, wherein the haematological cancer is a relapsed refractory haematological cancer.

20. The method according to claim 14, wherein the leukemia or myeloma is a relapsed and/or refractory leukemia or myeloma.
